(19) United States
(12) Patent Application Publication
(10) Pub. No.: US 2025/0375417 A1
(43) Pub. Date: Dec. 11, 2025

(54) OREXIN RECEPTOR 2 (OX2R) AGONIST FOR USE IN NARCOLEPSY TYPE 1 (NT1)

(71) Applicant: TAKEDA PHARMACEUTICAL COMPANY LIMITED, Osaka (JP)

(72) Inventors: Anson ABRAHAM, Cambridge, MA (US); Helene FAESSEL, Cambridge, MA (US); Yaming HANG, Cambridge, MA (US); Brian HAREL, Cambridge, MA (US); Marta KARAS, Cambridge, MA (US); Haruhide KIMURA, Fujisawa (JP); Elena KOUNDOURAKIS, Cambridge, MA (US); Tina OLSSON, Cambridge, MA (US); Christian VON HEHN, Cambridge, MA (US); Sebastian Philipp VON ROSENSTIEL, Cambridge, MA (US); Vikram SINHA, Cambridge, MA (US); Dmitri VOLFSON, Cambridge, MA (US); Hao WANG, Cambridge, MA (US); Andy ZHU, Cambridge, MA (US)

(21) Appl. No.: 19/195,036

(22) Filed: Apr. 30, 2025

Related U.S. Application Data
(60) Provisional application No. 63/641,320, filed on May 1, 2024.
(60) Provisional application No. 63/653,638, filed on May 30, 2024.
(60) Provisional application No. 63/675,599, filed on Jul. 25, 2024.
(60) Provisional application No. 63/690,651, filed on Sep. 4, 2024.
(60) Provisional application No. 63/697,871, filed on Sep. 23, 2024.

(51) Int. Cl. A61K 31/40; A61P 9/12; A61P 25/26; A61P 25/28

(52) U.S. Cl. CPC A61K 31/40; A61P 9/12; A61P 25/26; A61P 25/28

(57) ABSTRACT

Disclosed herein are methods, uses and composition for treating narcolepsy with cataplexy (narcolepsy type 1 or NT1), blunted nocturnal blood pressure dip in a subject having narcolepsy type 1 or cognitive impairment in a subject having narcolepsy type 1, by administering an orexin receptor 2 agonist to the subject at one or more daily dosages.

### CROSS-REFERENCES TO RELATED APPLICATIONS

This application claims the priority benefit of U.S. Provisional Application No. 63/641,320, filed May 1, 2024; U.S. Provisional Application No. 63/653,638, filed May 30, 2024; U.S. Provisional Application No. 63/675,599, filed Jul. 25, 2024; U.S. Provisional Application No. 63/690,651, filed Sep. 4, 2024; and U.S. Provisional Application No. 63/697,871, filed Sep. 23, 2024; which are each incorporated herein by reference in their entireties.

### TECHNICAL FIELD

This disclosure relates to methods of treating narcolepsy with cataplexy (narcolepsy type 1 or NT1) by administering an orexin receptor 2 agonist to the subject at one or more daily dosages.

### BACKGROUND

Narcolepsy is a rare neurologic disorder, whose cardinal symptom is excessive daytime sleepiness (EDS), described as a sudden overpowering need to sleep during the day's normal periods of alertness. Subjects with narcolepsy type 1 (NT1) also experience rapid eye movement (REM) phenomena that include cataplexy (sudden loss of muscle tone triggered by strong emotions).

The orexinergic system is a major wake-promoting system of the brain. It is comprised of wake-promoting orexin (also known as hypocretin) neurons, localized in a specific region of the lateral hypothalamus, which have excitatory projections to wide areas of the neuraxis including the cerebral cortex and other wake-promoting nuclei (cholinergic neurons of the basal forebrain, tuberomammillary nucleus, locus coeruleus, ventral tegmental area, and dorsal raphe nucleus). Narcolepsy type 1 has been defined by ICSD-3 or ICSD-3-TR criteria as having low levels of orexin in the cerebral spinal fluid (CSF) (≤110 pg/mL, or less than one-third of the laboratory's normal levels).

Two orexinergic neuropeptides, orexin A (OX-A) and orexin B (OX-B), have been identified to date. The OXs exert effects via 2 types of G-Protein-Coupled-Receptors, the orexin type 1 receptor (OX1R) and the orexin type 2 receptor (OX2R). OX-A has a high affinity to OX1R and OX2R, and OX-B has a high affinity to OX2R. The 2 OX receptors have a distinct distribution within various brain structures and monoaminergic arousal networks—the locus coeruleus contains only OX1R, and the tuberomammillary nucleus contains only OX2R. Because partial or complete OX deficiency plays an important role in the development of EDS, OX replacement therapy may improve EDS through a pathophysiology-directed mechanism of action.

The compound N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}ethanesulfonamide, (hereafter referred as to “Compound A”) is described in U.S. Pat. No. 11,028,048.

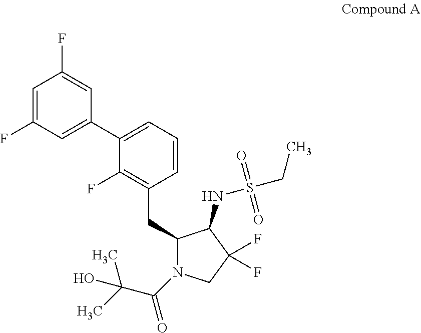 [SMILES: CCS(=O)(=O)N[C@@H]1[C@H](Cc2cccc(-c3cc(F)cc(F)c3)c2F)N(C(=O)C(C)(C)O)CC1(F)F]

Compound A is currently in clinical development as a therapy for narcolepsy type 1 (NT1).

The present disclosure provides methods of treating central disorders of hypersomnolence (CDH), in particular NT1, using the orexin type 2 receptor agonist Compound A.

### BRIEF DESCRIPTION OF THE DRAWINGS

FIG. 1 is a schematic representation of the Phase 2 trial described in Example 1. Abbreviations used in FIG. 1 are as follows: ABPM: ambulatory blood pressure monitoring; D: day; ePRO: electronic patient-reported outcome; FU: follow-up; LTE: long-term extension; MWT: Maintenance of Wakefulness Test; nPSG: nocturnal polysomnography; Outpt: outpatient; PK: pharmacokinetic(s).

FIG. 2 shows changes in sleep onset latency on the Maintenance of Wakefulness Test (MWT) versus placebo for the Phase 2 trial described in Example 1. Compound A resulted in significantly increased sleep onset latency on the MWT versus placebo. P-values shown have been adjusted for multiplicity. Footnotes: ¹Doghramji K., et al. Electroencephalogr Clin Neurophysiol 1997; 103:554-62. Abbreviations used in FIG. 2: LS: least squares; QD: once daily; MWT: Maintenance of Wakefulness Test.

FIG. 3 shows changes in Epworth Sleepiness Scale (ESS) total score versus placebo for the Phase 2 trial described in Example 1. Compound A resulted in significantly decreased ESS total score versus placebo. P-values shown have been adjusted for multiplicity. Footnotes: ¹Johns, M. W. Sleep 1991; 14:540-5. Abbreviations used in FIG. 3: ESS: Epworth Sleepiness Scale; LS: least squares; QD: once daily.

FIG. 4 shows percentage of participants with ESS≤10 at weeks 4 and 8 for the Phase 2 trial described in Example 1. Most participants achieved ESS total score≤10 with Compound A. Abbreviations used in FIG. 4: ESS: Epworth Sleepiness Scale.

FIG. 5 shows changes in median Weekly Cataplexy Rate (WCR) versus placebo for the Phase 2 trial described in Example 1. Twice daily dosing of Compound A significantly reduced cataplexy events compared with placebo. P-values shown have been adjusted for multiplicity. Abbreviations used in FIG. 5: IQR: interquartile range; QD: one daily; WCR: weekly cataplexy rate.

FIGS. 6A and 6B show changes in time in REM and rate of REM to wake/N1 transitions at baseline and after 8 weeks of treatment Compound A. FIG. 6A: (A) Montpellier study data shown as mean (95% CI) estimates for clinical controls and people with NT1. (B) Least-squares mean (95% CI) estimates for sleep quantities per quarter night. (C) Model contrasts of change from BL to week 8, where each coloured bar represents a quarter of the night. Reductions are significant when the CI does not contain zero. FIG. 6B: (A) Least-squares mean (95% CI) estimates for sleep quantities per quarter night. (B) Model contrasts of change from BL to week 8, where each coloured bar represents a quarter of the night. Transitions were normalised by time in REM and square-root transformed Reductions are significant when the CI does not contain zero. Abbreviations used in FIGS. 6A and 6B: BL: baseline.

FIG. 7 show the effects of Compound A on objective measurements of sleep using at-home continuous accelerometry. Week-by-week treatment effects of Cmpd. A shown as change from BL in (A) TST and (B) WASO. Participants may sleep as long as desired at home, unlike PSG nights where they are awoken on a schedule. The 2 mg/2 mg cohort showed small but statistically significant reductions in the number of nocturnal awakenings, number of nocturnal awakenings >5 min and longest sleep bout, with no significant changes observed in other groups. Results are significant when the CI does not contain zero. Abbreviations used in FIG. 7: BL: baseline.

FIG. 8 shows a 24-hour pattern approach for nocturnal BP dip calculation using an SBP example. SBP data for 1 subject with NT1 over 1 day (baseline ABPM measurement).

FIG. 9 shows the association between BP 24-hour pattern dip and sleep parameters. Effect estimate is the covariate's coefficient estimate from the linear mixed-effect model.

FIGS. 10A and 10B show the association of PVT with MTW (6A) and ESS (6B). PVT assessments at hour 1 and hour 7 post morning dose were averaged. FIG. 6A: MWT is weakly associated with attention in participants with NT1 (pooled across treatment arms). FIG. 6B: ESS is moderately associated with attention in participants with NT1 (pooled across treatment arms).

FIG. 11 shows the direct and indirect treatment effects of Compound A on PVT. Treatment effects on PVT with change in MWT as the mediator or change in ESS as the mediator show a large part of the improvement in sustained attention was a direct effect of Compound A. Data analyzed as change in square-root of PVT from baseline.

FIG. 12 shows the numerical correlation between sustained attention, disease severity, and activities of daily living. Pooled data for PVT lapses 1 h and 7 h post first dose were used for correlation calculations.

FIGS. 13A and 13B show the relationship between PVT lapses and ratings of global function, disease severity, quality of life, and activities of daily living. FIG. 13A-B Combined footnote: Responder definitions for CGI-I, PGI-I, NSS-CT, EQ-5D-5L: Improved (category went down at least one level), No change (category stayed the same), Worsened (category went up at least one level); FINI Social Activities: Responder=decrease of ≥40 on a 100-point scale; FINI Everyday Activities: Responder=decrease of ≥40 on a 100-point scale; FINI Everyday Responsibilities: Responder=decrease of ≥30 on a 100-point scale. No change and worsened categories were combined for CGI because of only 1 subject in the ‘worsened’ group and for NSS-CT because of only 2 subjects in the ‘no change’ group. PGI-I and EQ-5D-5L: pairwise t-test p-values. PVT lapses show average of 1-hour and 7-hours post-dose. Outliers are not displayed.

FIG. 14 shows the change in number of PVT lapses from baseline to 8 weeks of treatment with Compound A.

FIG. 15 shows the change in number of CPAL errors from baseline to 8 weeks of treatment with Compound A.

FIG. 16 shows the change in ONB performance speed from baseline to 8 weeks of treatment with Compound A. ONB performance speed results are log 10 transformed.

FIG. 17 shows the change in number of iDSST-s correct responses from baseline to 8 weeks of treatment with Compound A.

FIG. 18 shows Treatment Satisfaction Questionnaire for Medication (TSQM) domain scores for the Phase 2 trial described in Example 1. Global Satisfaction (Graph A) and Effectiveness (Graph B) domain scores were significantly higher across all dose groups of Compound A versus placebo (all P<0.001) and were consistent over time. The Convenience of treatment with Compound A was similar to placebo (Graph C). At week 8, impacts of Side Effects were similar for participants receiving twice-daily doses of Compound A and placebo, but significantly lower for participants receiving 7 mg of Compound A once daily versus placebo (Graph D). This analysis used a linear mixed-effects model for repeated measures with fixed effects for treatment, visit, and treatment-by-visit interaction. Abbreviations used in FIG. 18: LS: least squares; QD: once daily; TSQM: Treatment Satisfaction Questionnaire for Medication.

FIG. 19 shows results from exit interviews of participants in the Phase 2 trial described in Example 1 at Week 8, rating the change in the top 3 most important symptoms to improve. Not all patients provided a change rating for each symptom. * Numbers in this row represent the number of participants who indicated each of these symptoms were most important to improve. ^{†}Numbers in this row represent the number of participants who indicated that they experienced a meaningful change in each of the symptoms in this figure. Abbreviations used in FIG. 19: EDS: excessive daytime sleepiness.

FIG. 20 shows results from exit interviews of participants in the Phase 2 trial described in Example 1 at Week 8, rating the change in the top 3 functional impacts. Not all participants reported having each impact at baseline, and therefore did not provide ratings for change in all impacts. Abbreviations used in FIG. 20: QOL: quality of life.

FIG. 21 shows the change from baseline to Week 8 of the Phase 2 trial described in Example 1 in Functional Impacts of Narcolepsy Instrument (FINI) scores for the Social Activities domain (Graph A), Everyday Activities domain (Graph B) and Everyday Responsibilities domain (Graph C) domain. The analysis used a linear mixed-effects model for repeated measures, with fixed effects for baseline, treatment, visit, and treatment-by-visit interaction. Abbreviations used in FIG. 21: FINI: Functional Impacts of Narcolepsy Index; LS: least squares; QD: once daily.

FIG. 22 shows the change from baseline to Week 8 of the Phase 2 trial described in Example 1 in SF-36 summary scores for the Physical Component (Graph A) and the Mental Component (Graph B). Abbreviations used in FIG. 22: LS: least squares; SF-36:36-item Short Form; QD: once daily. Footnotes: ¹Maski, K. et al., J Clin Sleep Med 2021; 17:1895-945. The analysis used a linear mixed-effects model for repeated measures, with fixed effects for baseline, treatment, visit, and treatment-by-visit interaction.

FIG. 23 shows the change from baseline to Week 8 of the Phase 2 trial described in Example 1 in EuroQol-5 Dimensions 5-Levels (EQ-5D-5L) Index Score (Graph A) and Visual Analog Scale (VAS, Graph B). Abbreviations used in FIG. 23: LS: least squares; EQ-5D-5L: EuroQol-5 Dimension-5 Level; SE: standard error; QD: once daily; VAS: visual analog scale. The analysis used a linear mixed-effects model for repeated measures, with fixed-effects for baseline, treatment, visit, and treatment-by-visit interaction.

FIG. 24 shows changes in Narcolepsy Severity Scale for Clinical Trials (NSS-CT) scores for the Phase 2 trial described in Example 1. Abbreviations used in FIG. 24: CI: confidence interval; NSS-CT: Narcolepsy Severity Scale for Clinical Trials; QD: once daily; SE: standard error. The change from baseline analysis used a linear mixed-effects model for repeated measures, with fixed-effects for baseline, treatment, visit, and treatment-by-visit interaction.

FIG. 25 shows additional changes in NSS-CT scores for the Phase 2 trial described in Example 1. NSS-CT categories based on total score: Mild=0-14; Moderate=15-28; Severe=29-42; Very Severe=43-57. Abbreviations used in FIG. 25: NSS-CT: Narcolepsy Severity Scale for Clinical Trials; QD: once daily.

FIG. 26 shows changes in Clinical Global Impression-Severity (CGI-S) scores and Patient Global Impression-Severity (PGI-S) scores at Week 8 of the Phase 2 trial described in Example 1. Abbreviations used in FIG. 26: CGI-S: Clinical Global Impression-Severity; PGI-S: Patient Global Impression-Severity; QD: once daily

FIG. 27 shows changes in Clinical Global Impression-Improvement (CGI-I) scores and Patient Global Impression-Improvement (PGI-I) scores at Week 8 of the Phase 2 trial described in Example 1. Responders were participants who reported being much improved or very much improved. Abbreviations used in FIG. 27: CGI-I: Clinical Global Impression-Improvement; PGI-I: Patient Global Impression-Improvement; QD: once daily.

FIG. 28 is a schematic representation of the Phase 2 trial described in Example 2. Abbreviations used in FIG. 28: ABPM: ambulatory blood pressure monitoring; D: day; eDiary: electronic diary; ePRO: electronic patient-reported outcome; FU: follow-up; LTE: long-term extension; MWT: Maintenance of Wakefulness Test; nPSG: nocturnal polysomnography; Outpt: outpatient; PK: pharmacokinetic(s); PSG: polysomnography.

FIG. 29 shows the change in SOL-MWT in NT2 participants after 8 weeks of treatment with Compound A.

FIG. 30 shows the change in ESS total score in NT2 participants after 8 weeks of treatment with Compound A. Abbreviations used in FIG. 30: LS: least squares.

FIG. 31 is a summary of the MWT, ESS, WCR, and NSS-CT results at week 26 of the long term extension study described in Example 3A. The boxed section denotes the parent study period. Abbreviations used in FIG. 31: BL: baseline; QD: once daily.

### SUMMARY

In some aspects, the present disclosure provides a method of treating narcolepsy type 1 (NT1), the method comprising administering to a subject in need thereof about 1 mg to about 7 mg of Compound A:

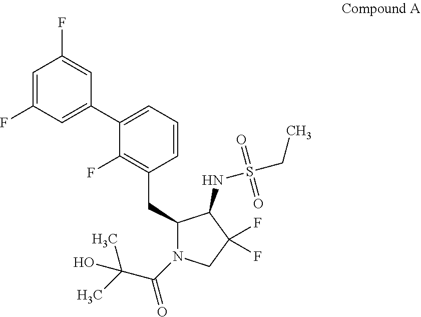 [SMILES: CCS(=O)(=O)N[C@@H]1[C@H](Cc2cccc(-c3cc(F)cc(F)c3)c2F)N(C(=O)C(C)(C)O)CC1(F)F]

or an equivalent amount of a pharmaceutically acceptable salt thereof. In some aspects, the subject in need of treating narcolepsy type 1 is administered two daily doses of Compound A, wherein each dose is from about 0.5 mg to about 5 mg, or an equivalent amount of a pharmaceutically acceptable salt thereof, and wherein the doses are administered about 2.5 hours to about 5.5 hours apart.

In some aspects, the present disclosure provides a method of treating blunted nocturnal blood pressure (BP) dip in a subject having narcolepsy type 1 (NT1), the method comprising administering to the subject about 1 mg to about 7 mg of Compound A:

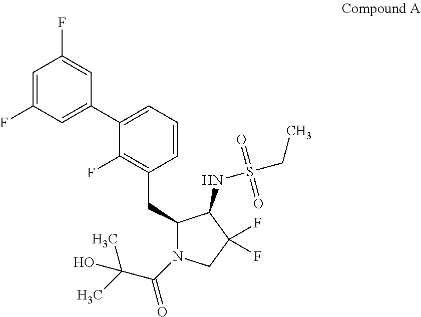 [SMILES: CCS(=O)(=O)N[C@@H]1[C@H](Cc2cccc(-c3cc(F)cc(F)c3)c2F)N(C(=O)C(C)(C)O)CC1(F)F]

or an equivalent amount of a pharmaceutically acceptable salt thereof. In some aspects, the subject in need of treating blunted nocturnal blood pressure is administered two daily doses of Compound A, wherein each dose is from about 0.5 mg to about 5 mg, or an equivalent amount of a pharmaceutically acceptable salt thereof, and wherein the doses are administered about 2.5 hours to about 5.5 hours apart.

In some aspects, the present disclosure provides a method of treating cognitive impairment in a subject having narcolepsy type 1 (NT1), the method comprising administering to the subject about 1 mg to about 7 mg of Compound A:

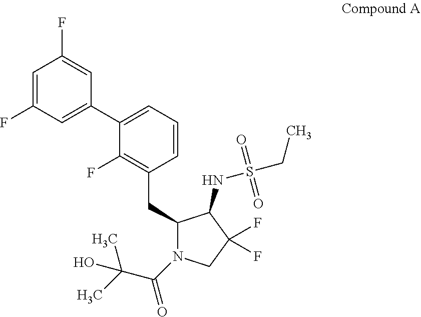 [SMILES: CCS(=O)(=O)N[C@@H]1[C@H](Cc2cccc(-c3cc(F)cc(F)c3)c2F)N(C(=O)C(C)(C)O)CC1(F)F]

or an equivalent amount of a pharmaceutically acceptable salt thereof. In some aspects, a subject in need of treatment for cognitive impairment is administered two daily doses of Compound A, wherein each dose is from about 0.5 mg to about 5 mg, or an equivalent amount of a pharmaceutically acceptable salt thereof, and wherein the doses are administered about 2.5 hours to about 5.5 hours apart.

In some aspects, the present disclosure provides Compound A:

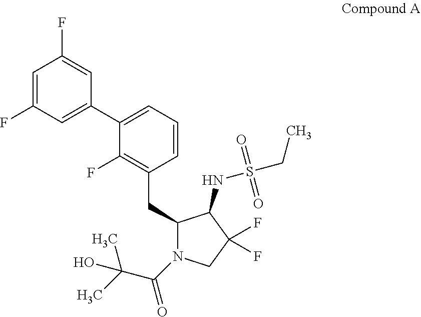 [SMILES: CCS(=O)(=O)N[C@@H]1[C@H](Cc2cccc(-c3cc(F)cc(F)c3)c2F)N(C(=O)C(C)(C)O)CC1(F)F]

or a pharmaceutically acceptable salt thereof for use in treating Narcolepsy Type 1 (NT1), wherein Compound A is to be administered in a daily dose of about 1 mg to about 7 mg of Compound A or an equivalent amount of a pharmaceutically acceptable salt thereof. In some aspects, two doses of Compound A are to be administered per day, wherein each dose is from about 0.5 mg to about 5 mg of Compound A or an equivalent amount of a pharmaceutically acceptable salt thereof, and wherein the doses are to be administered about 2.5 hours to about 5.5 hours apart.

In some aspects, the present disclosure provides Compound A:

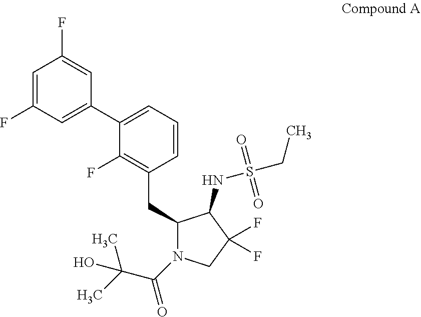 [SMILES: CCS(=O)(=O)N[C@@H]1[C@H](Cc2cccc(-c3cc(F)cc(F)c3)c2F)N(C(=O)C(C)(C)O)CC1(F)F]

or a pharmaceutically acceptable salt thereof for use in treating blunted nocturnal blood pressure (BP) dip in a subject having narcolepsy type 1 (NT1), wherein Compound A is to be administered in a daily dose of about 1 mg to about 7 mg of Compound A or an equivalent amount of a pharmaceutically acceptable salt thereof. In some aspects, two doses of Compound A are to be administered per day, wherein each dose is from about 0.5 mg to about 5 mg of Compound A or an equivalent amount of a pharmaceutically acceptable salt thereof, and wherein the doses are to be administered about 2.5 hours to about 5.5 hours apart.

In some aspects, the present disclosure provides Compound A:

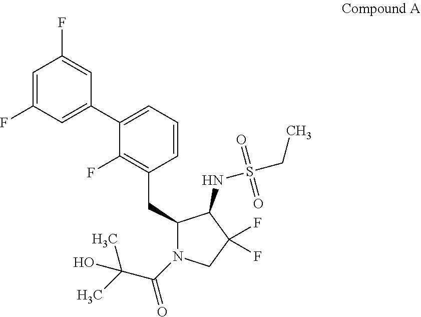 [SMILES: CCS(=O)(=O)N[C@@H]1[C@H](Cc2cccc(-c3cc(F)cc(F)c3)c2F)N(C(=O)C(C)(C)O)CC1(F)F]

or a pharmaceutically acceptable salt thereof for use in treating cognitive impairment in a subject having narcolepsy type 1 (NT1), wherein Compound A is to be administered in a daily dose of about 1 mg to about 7 mg of Compound A or an equivalent amount of a pharmaceutically acceptable salt thereof. In some aspects, two doses of Compound A are to be administered per day, wherein each dose is from about 0.5 mg to about 5 mg of Compound A or an equivalent amount of a pharmaceutically acceptable salt thereof, and wherein the doses are to be administered about 2.5 hours to about 5.5 hours apart.

In some aspects, the present disclosure provides a pharmaceutical composition for treating Narcolepsy Type 1 (NT1), which comprises (as a daily dose) about 1 mg to about 7 mg of Compound A:

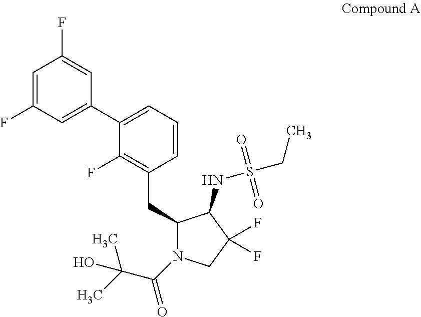 [SMILES: CCS(=O)(=O)N[C@@H]1[C@H](Cc2cccc(-c3cc(F)cc(F)c3)c2F)N(C(=O)C(C)(C)O)CC1(F)F]

or an equivalent amount of a pharmaceutically acceptable salt thereof. In some aspects, two doses of Compound A are to be administered per day, wherein each dose is from about 0.5 mg to about 5 mg of Compound A or an equivalent amount of a pharmaceutically acceptable salt thereof, and wherein the doses are to be administered about 2.5 hours to about 5.5 hours apart.

In some aspects, the present disclosure provides a pharmaceutical composition for treating blunted nocturnal blood pressure (BP) dip in a subject having narcolepsy type 1 (NT1), which comprises (as a daily dose) about 1 mg to about 7 mg of Compound A:

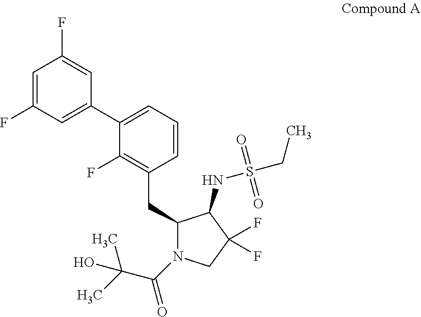 [SMILES: CCS(=O)(=O)N[C@@H]1[C@H](Cc2cccc(-c3cc(F)cc(F)c3)c2F)N(C(=O)C(C)(C)O)CC1(F)F]

or an equivalent amount of a pharmaceutically acceptable salt thereof. In some aspects, two doses of Compound A are to be administered per day, wherein each dose is from about 0.5 mg to about 5 mg of Compound A or an equivalent amount of a pharmaceutically acceptable salt thereof, and wherein the doses are to be administered about 2.5 hours to about 5.5 hours apart.

In some aspects, the present disclosure provides a pharmaceutical composition for treating cognitive impairment in a subject having narcolepsy type 1 (NT1), which comprises (as a daily dose) about 1 mg to about 7 mg of Compound A:

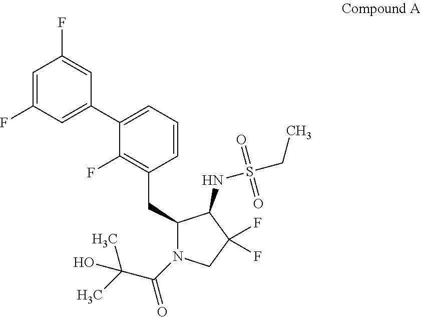 [SMILES: CCS(=O)(=O)N[C@@H]1[C@H](Cc2cccc(-c3cc(F)cc(F)c3)c2F)N(C(=O)C(C)(C)O)CC1(F)F]

or an equivalent amount of a pharmaceutically acceptable salt thereof. In some aspects, two daily doses of Compound A are to be administered per day, wherein each dose is from about 0.5 mg to about 5 mg of Compound A or an equivalent amount of a pharmaceutically acceptable salt thereof, and wherein the doses are to be administered about 2.5 hours to about 5.5 hours apart.

In some aspects of the methods, compounds for use or pharmaceutical compositions for use described herein, the two daily doses of Compound A, or the equivalent amount of a pharmaceutically acceptable salt thereof, are administered about 2.5 hours apart.

In some aspects of the methods, compounds for use or pharmaceutical compositions for use described herein, the two daily doses of Compound A, or the equivalent amount of a pharmaceutically acceptable salt thereof, are administered about 3 hours apart.

In some aspects of the methods, compounds for use or pharmaceutical compositions for use described herein, the two daily doses of Compound A, or the equivalent amount of a pharmaceutically acceptable salt thereof, are administered about 3.5 hours apart.

In some aspects of the methods, compounds for use or pharmaceutical compositions for use described herein, the two daily doses of Compound A, or the equivalent amount of a pharmaceutically acceptable salt thereof, are administered about 4 hours apart.

In some aspects of the methods, compounds for use or pharmaceutical compositions for use described herein, the two daily doses of Compound A, or the equivalent amount of a pharmaceutically acceptable salt thereof, are administered about 4.5 hours apart.

In some aspects of the methods, compounds for use or pharmaceutical compositions for use described herein, the two daily doses of Compound A, or the equivalent amount of a pharmaceutically acceptable salt thereof, are administered about 5 hours apart.

In some aspects of the methods, compounds for use or pharmaceutical compositions for use described herein, the two daily doses of Compound A, or the equivalent amount of a pharmaceutically acceptable salt thereof, are administered about 5.5 hours apart.

In some aspects of the methods, compounds for use or pharmaceutical compositions for use described herein, each of the two daily doses is about 1 mg to about 2 mg.

In some aspects of the methods, compounds for use or pharmaceutical compositions for use described herein, each of the two daily doses is about 1 mg.

In some aspects of the methods, compounds for use or pharmaceutical compositions for use described herein, each of the two daily doses is about 2 mg.

In some aspects of the methods, compounds for use or pharmaceutical compositions for use described herein, Compound A, or the pharmaceutically acceptable salt thereof, is administered orally.

In some aspects of the methods, compounds for use or pharmaceutical compositions for use described herein, Compound A, or the pharmaceutically acceptable salt thereof, is administered as a tablet.

In some aspects of the methods, compounds for use or pharmaceutical compositions for use described herein, Compound A, or the pharmaceutically salt thereof, is administered daily over a period of about 4 weeks to about 24 weeks.

In some aspects of the methods, compounds for use or pharmaceutical compositions for use described herein, Compound A, or the pharmaceutically salt thereof, is administered daily over a period of about 12 weeks.

In some aspects of the methods, compounds for use or pharmaceutical compositions for use described herein, Compound A, and not a pharmaceutically acceptable salt thereof, is administered to the subject.

In some aspects of the methods, compounds for use or pharmaceutical compositions for use described herein, the subject experiences a higher mean sleep latency from the Maintenance of Wakefulness Test (MWT) than baseline after a treatment period.

In some aspects of the methods, compounds for use or pharmaceutical compositions for use described herein, the subject experiences a higher mean sleep latency from the Maintenance of Wakefulness Test (MWT) after a treatment period compared to an untreated subject.

In some aspects of the methods, compounds for use or pharmaceutical compositions for use described herein, the subject experiences an increase in mean sleep latency in Maintenance of Wakefulness Test≥17 minutes compared to an untreated subject.

In some aspects of the methods, compounds for use or pharmaceutical compositions for use described herein, the subject experiences an increase in mean sleep latency in Maintenance of Wakefulness Test≥12 minutes compared to an untreated subject.

In some aspects of the methods, compounds for use or pharmaceutical compositions for use described herein, the subject experiences a lower Epworth Sleepiness Scale (ESS) total score after a treatment period compared to baseline.

In some aspects of the methods, compounds for use or pharmaceutical compositions for use described herein, the subject experiences a lower Epworth Sleepiness Scale (ESS) total score after a treatment period compared to an untreated subject.

In some aspects of the methods, compounds for use or pharmaceutical compositions for use described herein, the subject experiences an ESS statistically superior to an untreated subject with ESS placebo adjusted change of ≥8 and mean total endpoint score of ≤10.

In some aspects of the methods, compounds for use or pharmaceutical compositions for use described herein, the subject experiences an ESS statistically superior to an untreated subject with ESS placebo adjusted change of ≥8.

In some aspects of the methods, compounds for use or pharmaceutical compositions for use described herein, the subject experiences a lower weekly cataplexy rate (WCR) after a treatment period compared to baseline.

In some aspects of the methods, compounds for use or pharmaceutical compositions for use described herein, the subject experiences a lower weekly cataplexy rate (WCR) after a treatment period compared to an untreated subject.

In some aspects of the methods, compounds for use or pharmaceutical compositions for use described herein, the subject experiences a WCR>75% mean reduction from baseline which yields a statistically superior result compared to an untreated subject

In some aspects of the methods, compounds for use or pharmaceutical compositions for use described herein, the subject experiences a WCR>55% mean reduction from baseline which yields a statistically superior result compared to an untreated subject

In some aspects of the methods, compounds for use or pharmaceutical compositions for use described herein, the subject experiences a lower number of lapses on the psychomotor vigilance test (PVT) after a treatment period compared to baseline.

In some aspects of the methods, compounds for use or pharmaceutical compositions for use described herein, the subject experiences a lower number of lapses on the psychomotor vigilance test (PVT) after a treatment period compared to an untreated subject.

In some aspects of the methods, compounds for use or pharmaceutical compositions for use described herein, the subject experiences an improvement in overall severity of narcolepsy symptoms measured by the Narcolepsy Severity Scale for Clinical Trials (NSS-CT) score at Week 12 compared to baseline.

In some aspects of the methods, compounds for use or pharmaceutical compositions for use described herein, the subject experiences an improvement in overall severity of narcolepsy symptoms measured by the Narcolepsy Severity Scale for Clinical Trials (NSS-CT) score at Week 12 compared to an untreated subject.

In some aspects of the methods, compounds for use or pharmaceutical compositions for use described herein, the subject experiences improvement in functional impacts of narcolepsy as assessed by the Functional Impacts of Narcolepsy Instrument (FINI): FINI domain scores at Week 12 for Cognitive Functioning, Everyday Activities, and Everyday Responsibilities compared to baseline.

In some aspects of the methods, compounds for use or pharmaceutical compositions for use described herein, the subject experiences improvement in functional impacts of narcolepsy as assessed by the Functional Impacts of Narcolepsy Instrument (FINI): FINI domain scores at Week 12 for Cognitive Functioning, Everyday Activities, and Everyday Responsibilities compared to an untreated subject.

In some aspects of the methods, compounds for use or pharmaceutical compositions for use described herein, the subject experiences an improvement in quality of life of participants, as assessed by the Short Form-36 Survey (SF-36): SF-36 mental and physical component summary scores at Week 12 compared to baseline.

In some aspects of the methods, compounds for use or pharmaceutical compositions for use described herein, the subject experiences an improvement in quality of life of participants, as assessed by the Short Form-36 Survey (SF-36): SF-36 mental and physical component summary scores at Week 12 compared to an untreated subject.

In some aspects of the methods, compounds for use or pharmaceutical compositions for use described herein, the subject experiences an improvement in normalized REM architecture compared to baseline.

In some aspects of the methods, compounds for use or pharmaceutical compositions for use described herein, the subject experiences an improvement in normalized REM architecture compared to an untreated subject.

In some aspects of the methods, compounds for use or pharmaceutical compositions for use described herein, the subject experiences an increase after a treatment period in one or more of:
(a) systolic dip amplitude; and
(b) diastolic dip amplitude;
compared to an untreated subject.

In some aspects of the methods described herein, the subject experiences an increase after a treatment period in one or more of:
(a) systolic dip amplitude; and
(b) diastolic dip amplitude;
compared to baseline.

In some aspects of the methods, compounds for use or pharmaceutical compositions for use described herein, the subject experiences a reduction in attentional impairment compared to baseline.

In some aspects of the methods, compounds for use or pharmaceutical compositions for use described herein, the subject experiences a reduction in attentional impairment compared to an untreated subject.

In some aspects of the methods, compounds for use or pharmaceutical compositions for use described herein, the subject experiences a reduction in excessive daytime sleepiness (EDS) compared to baseline.

In some aspects of the methods, compounds for use or pharmaceutical compositions for use described herein, the subject experiences a reduction in excessive daytime sleepiness (EDS) compared to an untreated subject.

### DETAILED DESCRIPTION

The present disclosure provides methods of treating central disorders of hypersomnolence (CDH), in particular NT1. Narcolepsy is a rare, acquired, chronic neurologic disorder that alters the sleep-wake cycle. Five clinical features comprise the NT1 symptom pentad: EDS, cataplexy, hypnagogic/hypnopompic hallucinations, sleep paralysis, and disturbed nighttime sleep. Narcolepsy with cataplexy, or NT1, has been defined by the International Classification of Sleep Disorders, 3rd Edition (ICSD-3) or ICSD-3 text revision (ICSD-3-TR) criteria as having low levels of orexin (OX) in the cerebrospinal fluid (CSF) (≤110 μg/mL, or less than one-third of normal levels), most likely due to a selective loss of hypothalamic OX-producing neurons. An OX type-2 receptor (OX2R) agonist is thus the first approach to directly address the loss of OX peptide in the brain as it may restore OX2R signaling at the postsynaptic receptors and may be more effective than current therapies in treating the entire NT1 pentad, especially EDS and cataplexy.

In order that the present description can be more readily understood, certain terms are first defined. Additional definitions are set forth throughout the detailed description.

### I. Definitions

Unless otherwise stated, the following terms used in this application, including the specification and claims, have the definitions given below. It must be noted that, as used in the specification and the appended claims, the singular forms “a,” “an,” and “the” include plural referents unless the context clearly dictates otherwise. Unless otherwise indicated, conventional methods of pharmacology are employed. Furthermore, use of the term “including” as well as other forms, such as “include,” “includes,” and “included,” is not limiting. The section headings used herein are for organizational purposes only and are not to be construed as limiting the subject matter described.

All measurements are subject to experimental error and are within the spirit of the invention.

The term “about” as used in connection with a numerical value throughout the specification and the claims denotes an interval of accuracy, familiar and acceptable to a person skilled in the art. Such interval of accuracy is ±10%.

The term “acceptable” with respect to a formulation, composition or ingredient, as used herein, means having no persistent detrimental effect on the general health of the subject being treated.

The terms “administration of” and or “administering a” compound or composition should be understood to mean providing a compound or composition described herein to one or more subjects.

As used herein, the term “BP” refers to blood pressure, the term “SBP” refers to systolic blood pressure, the term “DBP” refers to diastolic blood pressure, and the term “ABPM” refers to ambulatory blood pressure monitoring.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “subject” and “participant” are used interchangeably and encompass mammals. Examples of mammals include, but are not limited to, humans, chimpanzees, apes, monkey, cattle, horses, sheep, goats, swine, rabbits, dogs, cats, rodents, rats, mice guinea pigs, and the like. In one aspect, the mammal is a human.

The term “baseline,” as used herein, refers to a clinical measurement or assessment of a particular subject prior to any of the treatments of this disclosure.

The terms “treat,” “treating,” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### II. Compound A

Compound A is described in U.S. Pat. No. 11,028,048. The chemical name for Compound A is N-{(2S,3R)-4,4-difluoro-1-(2-hydroxy-2-methylpropanoyl)-2-[(2,3′,5′-trifluoro[1, l′-biphenyl]-3-yl)methyl]pyrrolidin-3-yl}ethanesulfonamide

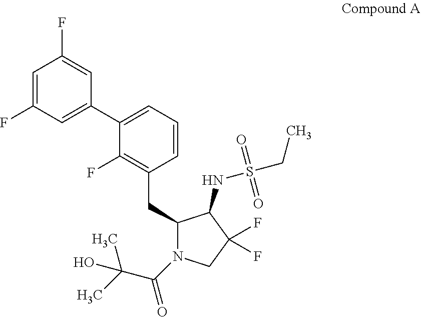 [SMILES: CCS(=O)(=O)N[C@@H]1[C@H](Cc2cccc(-c3cc(F)cc(F)c3)c2F)N(C(=O)C(C)(C)O)CC1(F)F]

### III. Compositions

In some aspects, the present disclosure provides compositions comprising Compound A. The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or ignore of the ingredient. Accordingly, the pharmaceutical compositions of the present invention encompass any composition made by mixing a compound of the present invention and a pharmaceutically acceptable carrier. By “pharmaceutically acceptable carrier” it is meant the carrier, diluent or excipient is compatible with the other ingredients of the formulation and not deleterious to the recipient thereof.

In some aspects, the compositions of the disclosure are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the compound of the disclosure can be in the form of a capsule. In some aspects, capsules can be immediate release capsules.

The compositions of the disclosure can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the compound of the disclosure can be in the form of a tablet. In some aspects, the compound of the disclosure can be in the form of a compressed tablet. In some aspects, the compound of the disclosure can be in the form of a film-coated compressed tablet. In some aspects, the compositions of the disclosure can be in the form of film-coated compressed tablets.

In some aspects, the compositions of the disclosure can be prepared by fluid bed granulation of the compound of the disclosure with one or more pharmaceutically acceptable carriers, vehicles, and/or excipients. In some aspects, the compositions of the disclosure can be prepared by fluid bed granulation process and can provide a tablet formulation with good flowability, good compressibility, fast dissolution, good stability, and/or minimal to no cracking. In some aspects, the fluid bed granulation process can allow preparation of formulations having high drug loading, such as over 70% or over 75% of a compound of the disclosure.

In some aspects, the compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, soft gelatin shells can contain a preservative to prevent the growth of microorganisms. Suitable preservatives include, but are not limited to, those as described herein, including methyl- and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. The capsules can also be coated as known by those of skill in the art in order to modify or sustain dissolution of the active ingredient.

In some aspects, the compositions of the disclosure can be in liquid or semisolid dosage forms, including emulsions, solutions, suspensions, elixirs, and syrups. In some aspects, the emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

In some aspects, the compositions of the disclosure for oral administration can be also provided in the forms of liposomes, micelles, microspheres, or nanosystems.

In some aspects, the compositions of the disclosure can be provided as non-effervescent or effervescent, granules and powders, to be reconstituted into a liquid dosage form. Pharmaceutically acceptable carriers and excipients used in the non-effervescent granules or powders can include, but are not limited to, diluents, sweeteners, wetting agents, and mixtures thereof. Pharmaceutically acceptable carriers and excipients used in the effervescent granules or powders can include, but are not limited to, organic acids, a source of carbon dioxide, and mixtures thereof.

Coloring and flavoring agents can be used in all of the above dosage forms. In addition, flavoring and sweetening agents can be especially useful in the formation of chewable tablets and lozenges.

In certain aspects, the compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

In certain aspects, the method comprises administering a pharmaceutically acceptable salt of Compound A. Examples of such salts include a salt with inorganic base, a salt with organic base, a salt with inorganic acid, a salt with organic acid, a salt with basic or acidic amino acid and the like. Examples of the salt with inorganic base include alkali metal salts such as sodium salt, potassium salt and the like, alkaline earth metal salts such as calcium salt, magnesium salt and the like, aluminum salt, ammonium salt and the like. Examples of the salt with organic base include salts with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine and the like. Examples of the salt with inorganic acid include salts with hydrochloric acid, hydrobromic acid, nitric acid, sulfuric acid, phosphoric acid and the like. Examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like. Examples of the salt with basic amino acid include salts with arginine, lysine, ornithine and the like. Examples of the salt with acidic amino acid include salts with aspartic acid, glutamic acid and the like.

In certain aspects, Compound A, or a pharmaceutically acceptable salt thereof, can be administered orally. In some aspects, Compound A, or a pharmaceutically acceptable salt thereof, can be administered in a capsule. In some aspects, Compound A, or a pharmaceutically acceptable salt thereof, can be administered in a tablet.

Compound A is typically administered in an admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, sucrose, dextrose, dextrates, glucose, maltodextrin, mannitol, xylitol, sorbitol, cyclodextrins, calcium phosphate, calcium sulfate, starches, modified starches, methyl cellulose, microcrystalline cellulose, microcellulose, talc and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, glidants, flavoring agents, and coloring agents can also be incorporated into the mixture.

In still other aspects, using standard coating procedures, such as those described in Remington's Pharmaceutical Sciences, 20th Edition (2000), a film coating is provided around the formulation of Compound A.

Dosage forms (pharmaceutical compositions) suitable for administration can contain from about 0.1 milligram to about 100 milligrams of active ingredient per dosage unit. In these pharmaceutical compositions the active ingredient will ordinarily be present in an amount of about 0.5-95% by weight based on the total weight of the composition. In some aspects, dosage forms suitable for administration can contain from about 0.1 to about 50 milligrams of active ingredient per dosage unit. In some aspects, dosage forms suitable for administration can contain from about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, or about 7.5 mg of active ingredient per dosage unit, or an equivalent amount of a pharmaceutically acceptable salt thereof.

In some aspects, the present disclosure provides pharmaceutical compositions which comprise Compound A as described herein, and at least one pharmaceutical acceptable carrier. Oral dosage forms, including tablets comprising Compound A are described in U.S. Pat. No. 11,028,048.

In some aspects, the pharmaceutical formulation for oral administration is a tablet.

In some aspects, the pharmaceutical formulation for oral administration is a tablet comprising a formulation described in Table 1:

TABLE 1
Compound A Tablet Formulations

| | | Quantity per Tablet (mg) | | |
|---|---|---|---|---|
| Components | Function | 0.5 mg | 1 mg | 2 mg |
| <Granulation> | | | | |
| Compound A | Active ingredient | 0.5 | 1 | 2 |
| Mannitol | Diluent | 80.5 | 80 | 79 |
| Microcrystalline | Diluent | 7 | 7 | 7 |
| Cellulose | | | | |
| Sodium Starch | Disintegrant | 5 | 5 | 5 |
| Glycolate Type A | | | | |
| Hydroxypropyl | Wet Binder | 3 | 3 | 3 |
| Cellulose | | | | |
| Purified Water ^{(a), (b)} | Solvent | q.s. | q.s. | q.s. |
| <Blending> | | | | |
| Microcrystalline | Diluent | 3 | 3 | 3 |
| Cellulose | | | | |
| Magnesium Stearate | Lubricant | 1 | 1 | 1 |
| Sub total | | 100 | 100 | 100 |
| <Film-coating | | | | |
| dispersion> | | | | |
| Hypromellose 2910 | Film coating agent | 3.77 | 3.77 | 3.77 |
| Polyethylene | Lubricant | 0.63 | 0.63 | 0.63 |
| Glycol 8000 | | | | |
| Calcium Carbonate | Coloring Agent | 0.68 | 0.68 | 0.68 |
| Ferric Oxide, Red | Coloring Agent | 0.012 | 0.04 | 0.12 |
| Ferric Oxide, Yellow | Coloring Agent | 0.108 | 0.08 | 0 |
| Purified Water ^{(a), (c)} | Solvent | q.s. | q.s. | q.s. |
| Total | | 105.2 | 105.2 | 105.2 |

q.s.: quantum sufficient;
USP: United States Pharmacopeia;
NF: National Formulary
^{(a)} Removed during the manufacturing process.
^{(b)} Binder solution concentration is 6 wt %.
^{(c)} Film-coating suspension concentration is 10 wt %.

In some aspects, the formulations described in Table 1 may be prepared as described in the process described in Table 2:

TABLE 1
Overview of Manufacturing and Bottle Packaging Process

| Step | Process | Description |
|---|---|---|
| 1 | Mannitol | Mannitol is milled through a screening mill equipped with a |
| | milling | suitable screen and weighted according to batch records. |
| 2 | Granulation | Compound A drug substance, screened mannitol, |
| | | microcrystalline cellulose, and sodium starch glycolate type A |
| | | are charged in a fluid bed granulator. The charged powders are |
| | | granulated by spraying the binder solution of hydroxypropyl |
| | | cellulose (HPC) in the fluid bed granulator. |
| 3 | Drying | The granules are dried in the granulator. |
| 4 | Milling | The dried granules are milled through a screening mill equipped |
| | | with a suitable screen. |
| 5 | Blending | The milled granules are blended with microcrystalline cellulose |
| | | and magnesium stearate in a diffusion mixer. |
| 6 | Compression | The blended granules are compressed into uncoated tablets with |
| | | a target weight, thickness, hardness, and disintegration time by |
| | | using a tablet press. |
| 7 | Film coating | An aqueous film coating dispersion of hypromellose 2910, |
| | | polyethylene glycol 8000, calcium carbonate, ferric oxide red, |
| | | and/or ferric oxide yellow is applied to the uncoated tablets in a |
| | | pan coating. The coating suspension is applied until the tablets |
| | | exhibit a target weight gain of 5.2 mg. After spraying, the |
| | | coated tablets are dried and cooled. |
| 8 | Packaging | The film-coated tablets are packed into a suitable container. |
| 9 | Primary | The film-coated tablets are packed into bottles. |
| | packaging | |

### IV. Methods of Treatment

The present disclosure provides methods of treating one or more central disorders of hypersomnolence (CDH) by administering Compound A, an orexin type 2 agonist. In some aspects, the one or more disorders is selected from narcolepsy type 1 (NT1), narcolepsy type 2 (NT2), and idiopathic hypersomnia. In some aspects, the one or more disorders is NT1.

Five clinical features comprise the NT1 symptoms pentad: excessive daytime sleepiness (EDS), cataplexy, hypnagogic/hypnopompic hallucinations, sleep paralysis, and disturbed nighttime sleep. In some aspects, the present disclosure provides methods of treating one or more NT1 symptoms selected from excessive daytime sleepiness (EDS), cataplexy, hypnagogic/hypnopompic hallucinations, sleep paralysis, and disturbed nighttime sleep. A suitable subject for treatment may be characterized by one or more of the following:
1) an ICSD-3 or ICSD-3-TR diagnosis of NT1 supported by results from 1) Polysomnography (PSG) (with or without Multiple Sleep Latency Test [MSLT]), performed within the past 15 years and meeting the minimal acceptable criteria for the proper performance of PSG/MSLT as outlined in the ICSD-3 or ICSD-3-TR, or 2) CSF test indicating an OX/hypocretin-1 concentration of ≤110 μg/mL (or less than one-third of the mean values obtained in normal participants within the same standardized assay);
2) an ICSD-3 diagnosis of NT2 by PSG/MSLT, performed within the past 5 years and meeting the minimal acceptable criteria for the proper performance of PSG/MSLT as outlined in the ICSD-3;
3) an ESS score≥11 at screening;
4) an ESS score>12 at screening;
5) ≥4 partial or complete episodes of cataplexy/week (WCR), calculated as the weekly average over 14 days (Days −16 to −3 of the screening period); or
6) a positive result for the HLA genotype HLA-DQB1*06:02 or results from radioimmunoassay indicate the participant's CSF OX/hypocretin-1 concentration is ≤110 μg/mL (or less than one-third of the mean values obtained in normal participants within the same standardized assay).

In certain aspects, the present disclosure provides a method of treating NT1, the method comprising administering to a subject in need thereof one or two doses of Compound A:

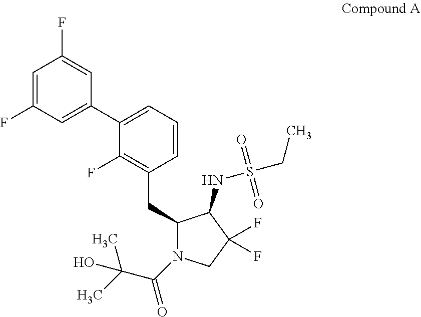 [SMILES: CCS(=O)(=O)N[C@@H]1[C@H](Cc2cccc(-c3cc(F)cc(F)c3)c2F)N(C(=O)C(C)(C)O)CC1(F)F]

or an equivalent amount of a pharmaceutically acceptable salt thereof. In some aspects, one dose of Compound A is administered. In some aspects, two doses of Compound A are administered.

In some aspects, the total amount of Compound A administered per day is from about 0.5 mg to about 10 mg, or an equivalent amount of a pharmaceutically acceptable salt thereof. In some aspects, the total amount of Compound A administered per day is from about 0.5 mg to about 7 mg, or an equivalent amount of a pharmaceutically acceptable salt thereof. In some aspects, the total amount of Compound A administered per day is from about 1 mg to about 7 mg, or an equivalent amount of a pharmaceutically acceptable salt thereof. In some aspects, the total amount of Compound A administered per day is about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, or an equivalent amount of a pharmaceutically acceptable salt thereof.

In some aspects, the method comprises administering from about 0.5 mg to about 10 mg of Compound A, or an equivalent amount of a pharmaceutically acceptable salt thereof, once per day. In some aspects, the method comprises administering from about 0.5 mg to about 7 mg of Compound A, or an equivalent amount of a pharmaceutically acceptable salt thereof, once per day. In some aspects, the method comprises administering from about 1 mg to about 7 mg of Compound A, or an equivalent amount of a pharmaceutically acceptable salt thereof, once per day. In some aspects, the method comprises administering about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of Compound A, or an equivalent amount of a pharmaceutically acceptable salt thereof, once per day. In some aspects, the method comprises administering about 1 mg, about 2 mg, about 4 mg, or about 7 mg of Compound A, or an equivalent amount of a pharmaceutically acceptable salt thereof, once per day.

In some aspects, the method comprises administering from about 0.25 mg to about 5 mg of Compound A, or an equivalent amount of a pharmaceutically acceptable salt thereof, twice per day. In some aspects, the method comprises administering from about 0.25 mg to about 3.5 mg of Compound A, or an equivalent amount of a pharmaceutically acceptable salt thereof, twice per day. In some aspects, the method comprises administering from about 0.25 mg to about 2.5 mg of Compound A, or an equivalent amount of a pharmaceutically acceptable salt thereof, twice per day. In some aspects, the method comprises administering about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, or about 5 mg of Compound A, or an equivalent amount of a pharmaceutically acceptable salt thereof, twice per day. In some aspects, the method comprises administering about 0.5 mg, about 1 mg, or about 2 mg of Compound A, or an equivalent amount of a pharmaceutically acceptable salt thereof, twice per day.

In some aspects, the method comprises administering two doses per day of Compound A, wherein each of the two doses is independently selected from about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, or about 5 mg, or an equivalent amount of a pharmaceutically acceptable salt thereof. In some aspects, the method comprises administering two doses of Compound A, wherein the doses are about 2 mg and about 5 mg, or equivalent amounts of pharmaceutically acceptable salts thereof.

In some aspects of the method, when two doses of Compound A, or a pharmaceutically acceptable salt thereof, are administered, the doses are administered about 1 to about 8 hours apart. In some aspects, the doses are administered about 1.5 to about 7 hours apart. In some aspects, the doses are administered about 2 to about 6 hours apart. In some aspects, the doses are administered about 2.5 to about 5.5 hours apart. In some aspects, the doses are administered about 2.5 to about 4 hours apart. In some aspects, the doses are administered about 1, about 1.5, about 2, about 2.5, about 3, about 3.5, about 4, about 4.5, about 5, about 5.5, about 6, about 6.5, about 7, or about 7.5, about 8 hours apart.

In some aspects, the method comprises administering from about 0.25 mg to about 5 mg of Compound A, or an equivalent amount of a pharmaceutically acceptable salt thereof, twice per day, wherein the two doses are administered about 1.5 to about 7 hours apart. In some aspects, the method comprises administering from about 0.25 mg to about 3.5 mg of Compound A, or an equivalent amount of a pharmaceutically acceptable salt thereof, twice per day, wherein the doses are administered about 3 to about 6 hours apart. In some aspects, the method comprises administering from about 0.25 mg to about 2.5 mg of Compound A, or an equivalent amount of a pharmaceutically acceptable salt thereof, twice per day, wherein the doses are administered about 2.5 to about 5.5 hours apart. In some aspects, the method comprises administering about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, or about 5 mg of Compound A, or an equivalent amount of a pharmaceutically acceptable salt thereof, twice per day, wherein the doses are administered about 2.5 to about 4 hours apart. In some aspects, the method comprises administering about 0.5 mg, about 1 mg, or about 2 mg of Compound A, or an equivalent amount of a pharmaceutically acceptable salt thereof, twice per day, wherein the doses are administered about 2.5, about 3, or about 3.5 hours apart.

In some aspects, the method comprises administering two doses per day of Compound A, wherein each of the two doses is independently selected from about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, or about 5 mg, or an equivalent amount of a pharmaceutically acceptable salt thereof, and wherein the doses are administered about 2.5 to about 4 hours apart. In some aspects, the method comprises administering two doses of Compound A, wherein the doses are about 2 mg and about 5 mg, or equivalent amounts of pharmaceutically acceptable salts thereof, and wherein the doses are administered about 2.5, about 3, or about 3.5 hours apart.

In some aspects, the method comprises administering Compound A, or a pharmaceutically acceptable salt thereof, daily over a period of about 4 weeks to about 24 weeks. In some aspects, the method comprises administering Compound A, or a pharmaceutically acceptable salt thereof, daily over a period of about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks.

In some aspects, the subject is administered Compound A, or a pharmaceutically acceptable salt thereof, with food. In some aspects, the subject is administered Compound A, or a pharmaceutically acceptable salt thereof, without food.

In some aspects of the methods described herein, the subject experiences a higher mean sleep latency (also referred to as sleep onset latency or SOL) from the Maintenance of Wakefulness Test (MWT) after a treatment period compared to an untreated subject. The MWT is a validated, objective measure that evaluates a person's ability to remain awake under soporific conditions for a defined period of time. Because there is no biological measure of wakefulness, wakefulness is measured indirectly by the inability or delayed tendency to fall asleep. This tendency to fall asleep is measured via electroencephalography-derived sleep latency in the MWT. One MWT, that includes four 40-minute wake trials, is done on each day specified in the Schedule of Activities of the trial. Sleep latency in each wake trial is recorded. Participants are required to stay awake in between the 4 MWT wake trials.

During each MWT wake trial, participants are instructed to sit in a bed or reclining chair and remain awake for as long as possible in a dimly lit room. Wake trials are ended after 40 minutes if no sleep occurs. If no sleep has been observed according to these rules, then the latency is defined as 40 minutes.

In some aspects of the methods described herein, the subject experiences a higher mean sleep latency from the Maintenance of Wakefulness Test (MWT) after a treatment period of about 4 weeks to about 24 weeks compared to an untreated subject. In some aspects of the methods described herein, the subject experiences a higher mean sleep latency from the Maintenance of Wakefulness Test (MWT) after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a higher mean sleep latency from the Maintenance of Wakefulness Test (MWT) after a treatment period of about 12 weeks compared to an untreated subject.

In some aspects of the methods described herein, the subject experiences an increase in mean sleep latency in Maintenance of Wakefulness Test≥20 minutes compared to an untreated subject (i.e., compared to placebo). In some aspects of the methods described herein, the subject experiences an increase in mean sleep latency in Maintenance of Wakefulness Test≥19 minutes compared to an untreated subject. In some aspects of the methods described herein, the subject experiences an increase in mean sleep latency in Maintenance of Wakefulness Test≥18 minutes compared to an untreated subject. In some aspects of the methods described herein, the subject experiences an increase in mean sleep latency in Maintenance of Wakefulness Test≥17 minutes compared to an untreated subject. In some aspects of the methods described herein, the subject experiences an increase in mean sleep latency in Maintenance of Wakefulness Test≥16 minutes compared to an untreated subject. In some aspects of the methods described herein, the subject experiences an increase in mean sleep latency in Maintenance of Wakefulness Test≥15 minutes compared to an untreated subject. In some aspects of the methods described herein, the subject experiences an increase in mean sleep latency in Maintenance of Wakefulness Test≥14 minutes compared to an untreated subject. In some aspects of the methods described herein, the subject experiences an increase in mean sleep latency in Maintenance of Wakefulness Test≥13 minutes compared to an untreated subject. In some aspects of the methods described herein, the subject experiences an increase in mean sleep latency in Maintenance of Wakefulness Test≥12 minutes compared to an untreated subject. In some aspects of the methods described herein, the subject experiences an increase in mean sleep latency in Maintenance of Wakefulness Test≥11 minutes compared to an untreated subject. In some aspects of the methods described herein, the subject experiences an increase in mean sleep latency in Maintenance of Wakefulness Test≥10 minutes compared to an untreated subject.

In some aspects of the methods described herein, the subject experiences a lower Epworth Sleepiness Scale (ESS) total score after a treatment period compared to an untreated subject. The ESS is a subjective, self-administered scale that has been validated and used extensively as a key endpoint in studies in patients with narcolepsy to measure excessive daytime sleepiness (EDS). The ESS provides individuals with 8 different situations of daily life and asks them how likely they are to fall asleep in those situations (scored 0 to 3) and to try to imagine their likelihood of dozing even if they have not actually been in the identical situation; the scores are summed to give an overall score of 0 to 24. Higher scores indicate stronger subjective daytime sleepiness, and scores below 10 are considered to be within the reference range.

In certain aspects, the ESS is administered to assess sleep propensity on selected days indicated in the Schedule of Activities for the trial. Participants are asked to evaluate their subjective sleepiness based on recalling their most recent daily life experiences.

In some aspects of the methods described herein, the subject experiences a lower Epworth Sleepiness Scale (ESS) total score after a treatment period of about 4 weeks to about 24 weeks compared to an untreated subject. In some aspects of the methods described herein, the subject experiences a lower Epworth Sleepiness Scale (ESS) total score after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a lower Epworth Sleepiness Scale (ESS) total score after a treatment period of about 12 weeks compared to an untreated subject.

In some aspects of the methods described herein, the subject experiences an ESS statistically superior to an untreated subject (i.e., compared to placebo) with ESS placebo adjusted change of ≥8 and mean total endpoint score of ≤10. In some aspects of the methods described herein, the subject experiences an ESS statistically superior to an untreated subject with ESS placebo adjusted change of ≥8.

The Karolinska Sleepiness Scale (KSS) is a 10-item, Likert-type rating scale for assessing subjective sleepiness. This participant self-rating scale measures the subjective level of sleepiness at a particular time during the day. Subjects indicate which level on this scale best reflects the psychophysical state experienced at the moment. The KSS is helpful in assessing the changes in response to the effects of drugs and is a measure of situational sleepiness. It is sensitive to fluctuations in situational sleepiness and has been used in studies of shift work, jetlag, attention/performance, and driving abilities as well as in clinical settings. It can assess changes in response to environmental factors, circadian rhythm, and effects of drugs. The KSS has high validity, with KSS results being highly correlated to nocturnal PSG and behavioral variables (Kaida et al. 2006; Reyner and Horne 1998).

In some aspects, the KSS can be used to assess the subjective level of sleepiness shortly before administration of the PVT (described herein).

In some aspects of the methods described herein, the subject experiences a lower KSS score after a treatment period of about 4 weeks to about 24 weeks compared to an untreated subject. In some aspects of the methods described herein, the subject experiences a lower KSS score after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a lower KSS score after a treatment period of about 12 weeks compared to an untreated subject.

In some aspects of the methods described herein, the subject experiences a lower weekly cataplexy rate (WCR) after a treatment period compared to an untreated subject. Episodes of cataplexy are self-reported by subjects in their study diary (participants complete a daily e-diary to record self-reported total sleep time, cataplexy events, and sleep quality. In cases where the e-diary becomes unavailable, a site may use alternative methods to collect these data with approval from sponsor or designee) with time of occurrence and severity (including body location).

Cataplexy events are recorded for 14 consecutive days, starting after completion of washout of any anti-cataplexy medications and completed before Day-2 are considered for WCR study entry criterion. The total number of events/week (WCR) is calculated based on the number of cataplexy episodes averaged over 2 weeks, with at least 11 out of 14 days required to calculate the WCR.

In some aspects of the methods described herein, the subject experiences a lower weekly cataplexy rate (WCR) after a treatment period of about 4 weeks to about 24 weeks compared to an untreated subject. In some aspects of the methods described herein, the subject experiences a lower weekly cataplexy rate (WCR) after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a lower weekly cataplexy rate (WCR) after a treatment period of about 12 weeks compared to an untreated subject.

In some aspects of the methods described herein, the subject experiences a WCR>75% mean reduction from baseline which yields a statistically superior result to an untreated subject (i.e., compared to placebo). In some aspects of the methods described herein, the subject experiences a WCR>55% mean reduction from baseline which yields a statistically superior result to an untreated subject.

Blunted nocturnal blood pressure (BP) dip, a potential risk factor for cardiovascular disease, may affect approximately ⅓ of patients with NT1. In the studies described herein, ambulatory blood pressure monitoring (ABPM) recordings can be obtained over an approximately 24.5-hour period and participants should not be confined to the clinical site during most of the 24.5-hour recording period. The ABPM device should be applied on-site. Because it is a requirement that the ABPM devices begin recording in the morning before the first morning dose (or similar time if performed during screening period), site staff may opt to confine participants in the evening before fitting participants with the ABPM monitoring device. If the participant is travelling to the site for the ABPM fitting, they may delay their first morning dose of study medication (to be taken no later than 10 AM) if needed. Participants who are confined the evening prior should also take their study drug right after the ABPM recording starts. For the ABPM during the screening period, the recording should begin between 8:00 AM±2 hours. Sites should ensure that all recordings meet the minimum threshold of acceptability. If a recording does not meet this threshold the site should collect a new recording within the protocol-specified time frame (avoiding nights immediately before nPSG or MWT). Participants will be trained on the use of the device with NT1.

Using functional data analysis, systolic BP (SBP) and diastolic BP (DBP) can be estimated in 24-hour patterns and key parameters, such as average, waveform amplitude, lowest and highest points, and dip amplitude (defined as [average−lowest]/average) can be extracted for each subject-visit. Non-dippers are defined as DBP 24-hour dip<13.44, as projected from a DBP fixed-window daytime to nighttime dip of <10%.

In certain aspects of the methods described herein, the subject experiences an improvement in their blunted nocturnal blood pressure dip.

In some aspects of the methods described herein, the subject experiences an increase over a treatment period in one or more of: (a) mean 24-hour systolic blood pressure; (b) mean 24-hour diastolic blood pressure; (c) mean systolic blood pressure; (d) mean diastolic blood pressure; (e) systolic dip amplitude; and (f) diastolic dip amplitude compared to an untreated subject. In some aspects of the methods described herein, the subject experiences said increase after a treatment period of about 4 weeks to about 24 weeks compared to an untreated subject. In some aspects of the methods described herein the subject experiences said increase after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences said increase after a treatment period of about 12 weeks compared to an untreated subject

Cognitive impairment may be identified from poor performance on validated, standardized, and objective tests of cognition. Since different tests may be used to measure different domains of cognition, the criteria for classification of impairment on each should be selected by the clinician on the basis of the tests used in their clinical context, their social context, and geographic area. Cognitive impairment, particularly in attentional function, is a common and disruptive symptom of NT1 characterized by deficits in one or more cognitive domains: attention, memory, and aspects of executive function, with the ability to sustain attention most affected by NT1. For purposes of the disclosure, cognitive impairment in these domains may be measured by one or more of the following tests:
psychomotor vigilance test (PVT) measures sustained attention (attention);
CPAL measures associate learning (memory);
ONB measures working memory (executive function); and
iDSST measures processing speed (executive function).

The psychomotor vigilance test (PVT) is validated as a clinical outcome assessment of the ability to sustain attention. The PVT is a simple reaction performance task that aims to measure sustained attention with no learning effects over repeated administration. Differences between healthy controls and patients with narcolepsy have been found on PVT performance for reaction time, accuracy, and other measures. The PVT is sensitive to the effects of total sleep loss, partial sleep restriction, naps, and circadian variation in the healthy population. The duration of test is 10 minutes.

The extent to which cognitive impairment is dependent on excessive daytime sleepiness (EDS) is unclear. Based on the clinical studies described herein, PVT lapses were not associated with MWT sleep latency but were associated moderately with ESS total score. Throughout the day, MWT sleep latency showed a slight decline, whereas PVT lapses remained stable. At week 8, Compound A improved attention and change from baseline in EDS (defined using MWT and ESS). The results of the trial suggest that attentional impairment can be dissociated from EDS, and both can be improved by Compound A. This indicates impairment in attention may arise directly from disordered disease biology, as well as indirectly from sleepiness.

In some aspects of the methods described herein, the subject experiences a lower number of lapses on the psychomotor vigilance test (PVT) after a treatment period compared to an untreated subject. In some aspects of the methods described herein, the subject experiences a lower number of lapses on the psychomotor vigilance test (PVT) after a treatment period of about 4 weeks to about 24 weeks compared to an untreated subject. In some aspects of the methods described herein, the subject experiences a lower number of lapses on the psychomotor vigilance test (PVT) after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a lower number of lapses on the psychomotor vigilance test (PVT) after a treatment period of about 12 weeks compared to an untreated subject.

In some aspects of the methods described herein, the subject experiences a superiority to an untreated subject (i.e., compared to placebo) on PVT, an aspect of cognition.

Improvements in sustained attention can be associated with improvements on assessments of global function, disease severity, and activities of daily living (ADLs) in patients with NT1. These assessments can be measured through the global impression of improvement of disease severity rated by clinicians (CGI-I) and patients (PGI-I), change on patient-rated disease severity (Narcolepsy Severity Scale, NSS-CT), general health (EuroQol 5D-5L, Usual Activities), and ADLs using three domains of the Functional Impacts of Narcolepsy Instrument (FINI; social activities, everyday activities and everyday responsibilities domains).

The Patient Global Impression Scale-Severity Scale (PGI-S) provides a participant-perceived assessment of overall symptoms (4-point Likert-type scale, “normal to “severe”) administered at baseline while the Patient Global Impression-Improvement Scale (PGI-I) provides a participant-perceived summary of global treatment effect on daytime sleepiness and cataplexy (7-point Likert-type scale, “very much improved” to “very much worse”) administered at different points during treatment.

The Clinical Global Impression Scale provides an overall clinician-determined summary measure of a participant's treatment experience that takes into account all available information, including knowledge of the participant's history, psychosocial circumstances, symptoms, behavior, and the impact of the symptoms on the participant's ability to function. The assessment consists of various subscales. One is the Clinical Global Impression-Severity (CGI-S). The CGI-S assesses the severity of overall narcolepsy symptoms. It uses a 7-point Likert-type scale ranging from “normal, not at all ill” to “among the most extremely ill patients.”

Another subscale is the Clinical Global Impression-Improvement (CGI-I), which assesses improvement of symptoms. The CGI-I asks clinicians to rate the extent to which their patients' current overall narcolepsy symptoms are improved (compared with the start of the study). This assessment uses a 7-point Likert-type scale ranging from “very much improved” to “very much worse”. Clinicians are trained on what aspects to take into consideration when rating the CGI-I to ensure consistency of responses.

Another subscale is the Clinical Global Impression-Change (CGI-C) which assesses change of symptoms. The CGI-C asks clinicians to rate the extent to which their patients' current overall narcolepsy symptoms are improved (compared to baseline). This assessment uses a 7-point Likert-type scale ranging from “very much improved” to “very much worse”. Clinicians are trained on what aspects to take into consideration when rating the CGI-C to ensure consistency of responses.

The Patient Global Impression rating scales capture global effect of treatment on daytime sleepiness, and overall narcolepsy symptoms as perceived by the participant. The Patient Global Impression-Severity (PGI-S) requires the participant to rate his/her disease severity at the time of assessment on a 5-point scale ranging from “none” to “very severe.” The PGI-C measures change due to treatment relative to baseline on a 7-point scale ranging from “very much improved” to “very much worse”. Instructions are provided to the participant to help ensure consistency in the tests.

In some aspects of the methods described herein, the subject experiences a greater improvement in global functioning after a treatment period compared to an untreated subject. In some aspects of the methods described herein, the subject experiences a greater improvement in global functioning after a treatment period of about 4 weeks to about 24 weeks compared to an untreated subject. In some aspects of the methods described herein, the subject experiences a greater improvement in global functioning after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a greater improvement in global functioning after a treatment period of about 12 weeks compared to an untreated subject.

Severity of NT1 symptoms can be assessed through the use of the NSS-CT. The NSS-CT is a 15-item self-administered questionnaire (total score: 0-57) that assesses the severity and consequences of the 5 major narcolepsy symptoms such as daytime sleepiness, cataplexy, hallucinations, sleep paralysis, and disrupted nighttime sleep (DNS). In a study conducted by Dauvilliers et al, the authors investigated the validity of the NSS and found that the mean symptom number, NSS total score, and number of narcolepsy symptoms were statistically significantly different between treated and untreated patients. Further, the symptom number was associated with diagnosis delay, age at onset, and ESS and Beck Depression Inventory scores (Dauvilliers et al. 2020).

In some aspects of the methods described herein, the subject experiences an improvement in overall severity of narcolepsy symptoms measured by the Narcolepsy Severity Scale for Clinical Trials (NSS-CT) score at Week 12.

In some aspects of the methods described herein, the subject experiences a decrease in the severity of NT1 symptoms after a treatment period compared to an untreated subject. In some aspects of the methods described herein, the subject experiences a decrease in the severity of NT1 symptoms after a treatment period of about 4 weeks to about 24 weeks compared to an untreated subject. In some aspects of the methods described herein, the subject experiences a decrease in the severity of NT1 symptoms after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a decrease in the severity of NT1 symptoms after a treatment period of about 12 weeks compared to an untreated subject.

Health conditions such as mobility, self-care, usual activities, pain/discomfort, and/or anxiety/depression as related to NT1 can be assessed by the EQ-5D-5L assessment. The EQ-5D-5L is a standardized 5-item measure of health status developed by the EuroQol Group to provide a simple, generic measure of health for clinical and economic appraisal that has been used in a wide range of health conditions and treatments (Herdman et al. 2011; Janssen et al. 2013). The EQ-5D-5L consists of a descriptive system and the EuroQol visual analog scale (VAS). The descriptive system comprises 5 dimensions: mobility, self-care, usual activities, pain/discomfort, and anxiety/depression. The EuroQol VAS records the participant's self-rated health on a vertical VAS. This can be used as a quantitative measure of health outcome that reflects the participant's own judgment. The scores on these 5 dimensions can be presented as a health profile or can be converted to a single summary index number (utility) reflecting preferability compared with other health profiles.

In some aspects of the methods described herein, the subject experiences a greater improvement in mobility, self-care, usual activities, pain/discomfort, and/or anxiety/depression after a treatment period compared to an untreated subject. In some aspects of the methods described herein, the subject experiences a greater improvement in mobility, self-care, usual activities, pain/discomfort, and/or anxiety/depression after a treatment period of about 4 weeks to about 24 weeks compared to an untreated subject. In some aspects of the methods described herein, the subject experiences a greater improvement in mobility, self-care, usual activities, pain/discomfort, and/or anxiety/depression after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a greater improvement in mobility, self-care, usual activities, pain/discomfort, and/or anxiety/depression after a treatment period of about 12 weeks compared to an untreated subject.

The functional impact of NT1 on subjects can be assessed by the FINI. The FINI is a fit-for-purpose 28-item instrument developed by the sponsor to ensure easy to interpret concepts that are meaningful to narcolepsy patients. The FINI measures the functional impacts of narcolepsy across 6 domains: Tiredness (items 1-7), Cognitive Functioning (items 8-12), Cataplexy (items 13-17), Social Activities (items 18-21), Everyday Activities (items 22-25), and Everyday Responsibilities (items 26-28). Each item asks about the impact that narcolepsy has had on their daily functioning during the past 7 days, and scored from 0-4, where 0 indicates the best health and 4 the worst. There is no total score; for each participant, an average score is calculated for each domain, if at least half the items in the domain have been answered:

Sum of responses in the domain/Number of completed items in the domain

The average domain score is then standardized to a 0-100 scale, where 0 indicates the best health and 100 the worst health. A decrease from baseline would thus indicate improved health:

Standardized score=average score/4×100

For example, if the 4 items in one domain (eg, Social Activities) had the responses 1, 2, 3, 2, the average score would be 2 (calculated as (1+2+3+2)/4) and the standardized score would be 50 points (calculated as 2/4×100).

In some aspects of the methods described herein, the subject experiences an improvement in functional impacts of narcolepsy as assessed by the Functional Impacts of Narcolepsy Instrument (FINI): FINI domain scores at Week 12 for Cognitive Functioning, Everyday Activities, and Everyday Responsibilities compared to baseline. In some aspects of the methods described herein, the subject experiences an improvement in functional impacts of narcolepsy as assessed by the Functional Impacts of Narcolepsy Instrument (FINI): FINI domain scores at Week 12 for Cognitive Functioning, Everyday Activities, and Everyday Responsibilities compared to an untreated subject (i.e., compared to placebo).

In some aspects of the methods described herein, the subject experiences a lesser impact of narcolepsy on their daily functioning after a treatment period compared to an untreated subject. In some aspects of the methods described herein, the subject experiences a lesser impact of narcolepsy on their daily functioning after a treatment period of about 4 weeks to about 24 weeks compared to an untreated subject. In some aspects of the methods described herein, the subject experiences a lesser impact of narcolepsy on their daily functioning after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a lesser impact of narcolepsy on their daily functioning after a treatment period of about 12 weeks compared to an untreated subject.

Treatment satisfaction as it pertains to NT1 treatments can be assessed by the TSQM. The TSQM vII is a 11-item generic patient-reported outcome assessment that measures satisfaction with treatment in 4 key dimensions: effectiveness (items 1-2), side effects (items 3-6), convenience (items 7-9), and global satisfaction (items 10-11) (Atkinson et al. 2005). Responses within each domain to these 11 items are summed and transformed on a 0-100 score, with higher scores indicating better treatment satisfaction.

In some aspects of the methods described herein, the subject experiences a better treatment satisfaction after a treatment period compared to an untreated subject. In some aspects of the methods described herein, the subject experiences a better treatment satisfaction after a treatment period of about 4 weeks to about 24 weeks compared to an untreated subject. In some aspects of the methods described herein, the subject experiences a better treatment satisfaction after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a better treatment satisfaction after a treatment period of about 12 weeks compared to an untreated subject.

Patient health self-assessments can be measured by the SF-36. The SF-36 is a 36-item, participant-reported survey of participant health. The SF-36 consists of 8 scaled scores (vitality, physical functioning, bodily pain, general health perceptions, physical role functioning, emotional role functioning, social role functioning, mental health), which are the weighted sums of the questions in their section. Each scale is directly transformed into a 0 to 100 scale on the assumption that each question carries equal weight. The lower the score the more disability. The higher the score the less disability, that is, a score of zero is equivalent to maximum disability and a score of 100 is equivalent to no disability. When scores for all 8 scales are available, 2 summary measures known as component scores are derived: the Physical Health Component Score and the Mental Health Component Score. Similar to each scale, the component scores are also positively scored so that higher scores represent better health-related quality-of-life.

In some aspects of the methods described herein, the subject experiences an improvement compared to baseline in quality of life as assessed by the Short Form-36 Survey (SF-36): SF-36 mental and physical component summary scores at Week 12. In some aspects of the methods described herein, the subject experiences an improvement compared to an untreated subject (i.e., compared to placebo) in quality of life as assessed by the Short Form-36 Survey (SF-36): SF-36 mental and physical component summary scores at Week 12.

In some aspects of the methods described herein, the subject experiences a better health-related quality-of-life after a treatment period compared to an untreated subject. In some aspects of the methods described herein, the subject experiences a better health-related quality-of-life after a treatment period of about 4 weeks to about 24 weeks compared to an untreated subject. In some aspects of the methods described herein, the subject experiences a the subject experiences a better health-related quality-of-life after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a better health-related quality-of-life after a treatment period of about 12 weeks compared to an untreated subject.

NT1 cognitive impairment is also characterized by deficits in memory and aspects of executive function. The CPAL test is a measure of visual associate memory and uses a well-validated paired associate learning paradigm in which the participant must learn the locations of a number of amoeba-like shapes on the computer screen. This test consists of a single amoeboid shape displayed in the center of the screen surrounded by a number of blue-filled circles. In the exposure phase of the test all of the to-be-remembered pattern-location associations are presented on the computer screen simultaneously. After a 5-second delay, a pattern is shown in the central location and this signals that the participant should touch the location in the periphery that contains the same pattern. This process continues until the participant has acknowledged all of the pattern-location associations. The learning phase begins with the same test display presented during the exposure phase except that now all of the peripheral locations are shown as blue spheres. One of the patterns presented in the exposure phase is presented in the center location. With the presentation of this pattern, the participant is required to select the peripheral location where an identical pattern is hidden beneath the blue sphere. This process continues until the correct location of each pattern is found. Finding the correct location for all patterns in the set is defined as a learning trial. The software records each move as an error or as a correct move. The duration of the test is 7 minutes.

The ONB test is a measure of working memory and uses a well-validated n-back paradigm with playing card stimuli. In this test, the playing cards are identical to those found in a standard deck of 52 playing cards (without the joker cards). The participant is asked whether the card displayed in the center of the screen is the same as the card presented immediately previously. The participant responds by pressing the Yes or No key. Because no card has been presented yet on the first trial, a correct first response is always No. The software measures the speed and accuracy of each response. The duration of the test is 4 minutes.

The iDSST-s is a processing speed test that is based on the pre-existing pencil and paper version of the Digit Symbol Substitution Test. In this test, participants are presented with a legend that defines 9 symbols, with each symbol corresponding to a digit from 1 to 9. The participant is then presented with a conveyer belt in the middle of the screen that displays a series of empty boxes labelled with a number. The participant must select the symbol that corresponds to the number of a given highlighted box from symbol options presented at the bottom of the screen. The participant must try to place as many correct symbols in the boxes as possible over the duration of the test. Performance is measured by calculating the total number of correct responses. The duration of the test is 3 minutes.

In some aspects of the methods described herein, the subject experiences a greater improvement in memory and aspects of executive function after a treatment period compared to an untreated subject. In some aspects of the methods described herein, the subject experiences a greater improvement in memory and aspects of executive function after a treatment period of about 4 weeks to about 24 weeks compared to an untreated subject. In some aspects of the methods described herein, the subject experiences a greater improvement in memory and aspects of executive function after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a greater improvement in memory and aspects of executive function after a treatment period of about 12 weeks compared to an untreated subject.

Effects of NT1 on a subject's work life can be assess using the WPAI: SHP. The WPAI: SHP v2.0 questionnaire is a 6-item self-administered questionnaire that measures work time missed and work and activity impairment because of a specified health problem during the past 7 days. The WPAI: SHP will be used with “narcolepsy” as the specified health problem. The validity of the WPAI: SHP has been established in a number of diseases (Reilly 1993). The instrument has 6 questions and yields 4 scores: absenteeism, presenteeism, work productivity loss, and activity impairment outside of work due to a specified health problem. Work impairment will be evaluated among employed participants and overall activity impairment will be assessed among all participants. For each score, the corresponding WPAI: SHP outcome is expressed as a percentage, with higher numbers indicating greater impairment and less productivity.

In some aspects of the methods described herein, the subject less missed worktime and/or lower work impairment after a treatment period compared to an untreated subject. In some aspects of the methods described herein, the subject less missed worktime and/or lower work impairment after a treatment period of about 4 weeks to about 24 weeks compared to an untreated subject. In some aspects of the methods described herein, the subject less missed worktime and/or lower work impairment after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject less missed worktime and/or lower work impairment after a treatment period of about 12 weeks compared to an untreated subject.

The BC-CCI-E is a test assessing perceived cognitive difficulties and the functional and quality of life impacts of cognitive complaints. The BC-CCI scale consists of 6 items assessing perceived problems with concentration, memory, expressing thoughts, word finding, slow thinking, and difficulty solving problems in the last 7 days. Scores on each item range from 0 (not at all, or false, not at all true) to 3 (very much, or very true) and are summed to yield a total score ranging from 0 to 18, with higher scores indicating greater impact of cognitive complaints. The expanded version includes 3 additional items to assess the functional and quality of life impacts of cognitive complaints. The duration of the test is less than 5 minutes.

In some aspects of the methods described herein, the subject experiences a fewer perceived cognitive difficulties after a treatment period compared to an untreated subject. In some aspects of the methods described herein, the subject experiences a fewer perceived cognitive difficulties after a treatment period of about 4 weeks to about 24 weeks compared to an untreated subject. In some aspects of the methods described herein, the subject experiences a fewer perceived cognitive difficulties after a treatment period of about 4, about 5, about 6, about 7, about 8, about 9, about 10, about 11, about 12, about 13, about 14, about 15, about 16, about 17, about 18, about 19, about 20, about 21, about 22, about 23, and/or about 24 weeks compared to an untreated subject. In some aspects of the methods described herein, the subject experiences a fewer perceived cognitive difficulties after a treatment period of about 12 weeks compared to an untreated subject.

### EXAMPLES

### Example 1. A Randomized, Double-Blind, Placebo-Controlled Study to Evaluate the Efficacy, Safety, and Tolerability of Compound a for the Treatment of Narcolepsy with Cataplexy (Narcolepsy Type 1)

The study was performed in approximately 100 (male and female) participants with NT1, who were randomized such that each participant had an equal chance of being assigned to any 1 of 5 treatment arms: 3 Compound A twice daily dose regimens, 1 Compound A once daily dose regimen, or matching placebo. Randomization was stratified by region. Starting on the morning of Day 1, the study drug was administered at approximately the same time each day for 8 or 12 weeks. If the participant wished to enroll in the long-term extension (LTE) study described in Example 3 but the LTE study had not started at the participant's site when the participant completed the Week 8 visit of the current study, the participant was allowed to continue in the current study for an additional 4 weeks.

Participants who provided informed consent completed a screening period of up to 50 days to washout any NT1 medication (if applicable). Participants were asked to complete an e-diary, starting from the initial screening visit, no later than Day-16. To be eligible for the study, participants had to fill out self-reported cataplexy questions in the e-diary for at least 11 days of the 14-day period from Day −16 to Day −3 and have had >4 partial and/or complete episodes of cataplexy/week (averaged over Days −16 to −3).

Participants remained confined overnight at the study site during the following times:
Days −2 to 1 (1 mandatory overnight at Day-2; 1 optional overnight at Day-1)
Days 27 to 28 (1 overnight)
Days 55 to 56 (1 overnight)

After the Week 8 visit, participants had the option to participate in an LTE study under a separate protocol (Example 3), assuming the protocol was open for enrollment. Participants who enrolled in the LTE study did not have follow-up visits in this study.

If a participant planned to enroll in the LTE study but the LTE study was not open for enrollment at the participant's site when the participant completed the Week 8 visit, the participant was allowed to continue in this study for an additional 4 weeks of treatment (continuing the same treatment regimen as the previous 8 weeks). This was to avoid a treatment gap for participants that were willing to roll over from this study to the LTE study.

For participants who did not participate in the LTE study (including any participant who completed the Week 12 visit and for any reason did not enroll in the LTE), every effort was deployed to have them complete a first follow-up visit approximately 7 days after the final study drug intake and a second follow-up visit (home healthcare visit) approximately 28 days after the final study drug intake. For participants who terminated the study early, every effort was deployed to have them complete an early termination visit as soon as possible and a follow-up visit (in-clinic visit or home healthcare, if available) approximately 28 days after the last dose of study drug. Participants not participating in the LTE study could restart their non-exclusionary medications after the first follow-up visit or early termination visit.

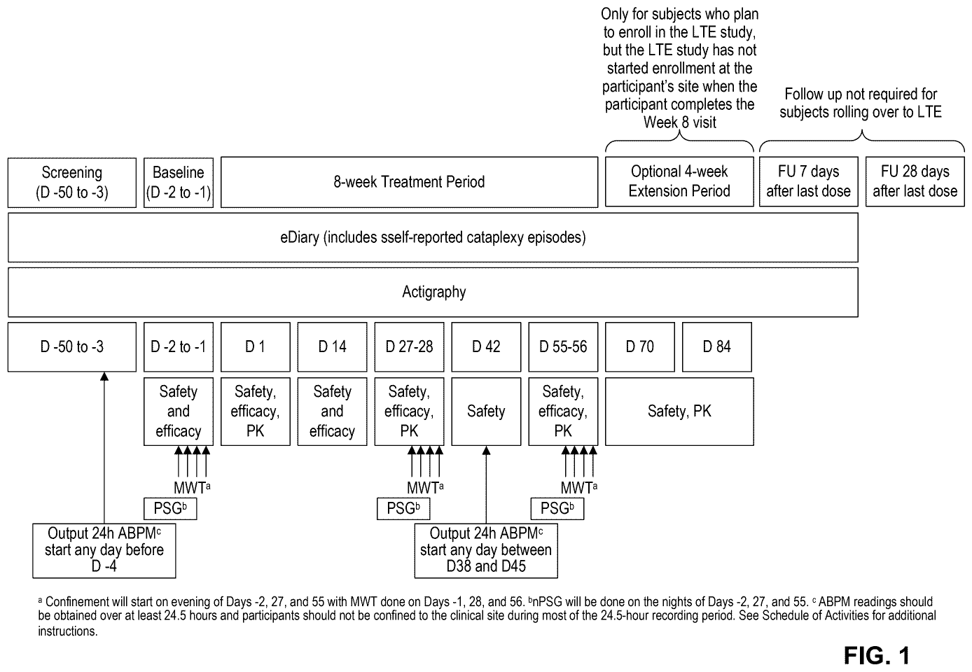

For a schematic of the study design, see FIG. 1.

Standard safety endpoints (e,g., treatment ending adverse events (TEAEs), physical examination findings, vital signs, 12-lead ECG measures, clinical laboratory results) for early clinical investigation were included. Further, because cardiovascular effects have been noted in nonclinical models with Compound A and OX2R agonists in general, and these effects are thought to be on-target effects, close monitoring of cardiovascular parameters, including time-matched BP measurements before and after dosing to detect any changes and potential tolerance in BP after the administration of repeat doses to participants with NT1 were included. Ambulatory blood pressure monitoring (ABPM) was also obtained to assess BP in the outpatient setting in a rigorous manner.

In addition, because liver function test (LFT) elevation cases have been observed in another OX2R agonist with structural similarity, LFTs (ALT, AST, GGT, and total bilirubin) were closely monitored.

### Type of Participant and Disease Characteristics

1. The participant was aged 18 to 70 years, inclusive, at the time of signing the informed consent form (16-70 in Japan).

2. The participant had body mass index (BMI) within the range 18 to 40 kg/m² (inclusive).

3. The participant had an ICSD-3 diagnosis of NT1 by polysomnography (PSG)/Multiple Sleep Latency Test (MSLT), performed within the past 10 years.

4. The participant had an ESS score>12 on Day −1.

5. The participant has >4 partial and/or complete episodes of cataplexy/week (WCR), calculated as the weekly average over 14 days (Days −16 to −3 of the screening period).

6. The participant was positive for the HLA genotype HLA-DQB1*06:02 or results from CSF testing indicated the participant's CSF OX/hypocretin-1 concentration was <110 μg/mL (or less than one-third of the mean values obtained in normal participants within the same standardized assay).

### Exclusion Criteria

1. The participant had a current medical disorder, other than narcolepsy with cataplexy, associated with EDS.

2. The participant had a current medical condition such as unstable cardiovascular, pulmonary, renal, or gastrointestinal disease that would preclude enrollment in the view of the investigator.

3. The participant had medically significant hepatic or thyroid disease.

4. The participant had current or recent (within 6 months) gastrointestinal disease that was expected to influence the absorption of drugs (i.e., a history of malabsorption, esophageal reflux, peptic ulcer disease, erosive esophagitis, frequent [more than once per week] occurrence of heartburn, or any surgical intervention).

5. The participant had a history of cancer in the past 5 years (does not apply to participants with carcinoma in situ that had been resolved without further treatment or basal cell cancer; these participants could be included after approval by the sponsor or designee).

6. The participant had clinically significant coronary artery disease, a history of myocardial infarction, clinically significant angina, clinically significant cardiac rhythm abnormality, or heart failure.

7. The participant had a clinically significant history of head injury or head trauma.

8. The participant had history of epilepsy, seizure, or convulsion, or had a family history of inherited disorders associated with seizure (except for a single febrile seizure in childhood).

9. The participant had one or more of the following psychiatric disorders:
a) Any current unstable psychiatric disorder.
b) Current or history of manic or hypomanic episode, schizophrenia or any other psychotic disorder, including schizoaffective disorder, major depression with psychotic features, bipolar depression with psychotic features, obsessive compulsive disorder, mental retardation, organic mental disorders, or mental disorders due to a general medical condition as defined in the Diagnostic and Statistical Manual of Mental Disorders, 5th Edition (DSM-5).
c) Current diagnosis or history of substance use disorder as defined in the DSM-5.

Note: If the history of substance use disorder was more than 12 months before baseline, the participant could be allowed to enroll in the study after consultation with the sponsor or designee. (Participant must also have negative urine drug screen at the screening and Day −2 visits.)

d) Current active major depressive episode (MDE) or who have had an active MDE in the past 6 months.

10. The participant had a history of cerebral ischemia, transient ischemic attack (<5 years ago), intracranial aneurysm, or arteriovenous malformation.

11. The participant had a positive test result for hepatitis B surface antigen, hepatitis C virus antibody, or HIV antibody/antigen at screening.

12. The participant's renal creatinine clearance (Cockcroft-Gault Equation) was ≤50 mL/min at screening.

13. The participant had ALT or AST values>1.5 times the upper limit of normal (ULN) at screening or Day −2 (if results available); or the participant had ALT or AST between 1.0 and 1.5×ULN at screening with ≥15% increase from screening to Day −2.

14. The participant was considered by the investigator to be at imminent risk of suicide or injury to self, others, or property, or the participant had attempted suicide within the past year before screening, or had positive answers on item number 4 or 5 on the C-SSRS (based on the past year) before randomization.

15. The participant is unable to refrain from or anticipates using excluded food products beginning by Day −7 and continuing until the first follow-up visit, or prohibited medication.

16. The participant has participated in another investigational drug study, in which they received the investigational drug, within 60 days (or 6 months if participant may have received an investigational biologic product). The interval window from the previous study will be derived from the date of the last study procedure in the previous study to the screening visit of the current study.

### Study Interventions Administered:

The following doses were studied:
Compound A Dose Regimen 1:0.5 mg twice daily approximately 3 hours apart.
Compound A Dose Regimen 2:2 mg twice daily approximately 3 hours apart.
Compound A Dose Regimen 3:2 mg followed by 5 mg approximately 3 hours apart.
Compound A Dose Regimen 4:7 mg once daily (QD).
Other products required for the study: Matching placebo.

Dose regimens included once daily (QD) or twice daily (approximately 3 hours apart) dosing. Study treatment was administered at approximately 8 AM and 11 AM. (Participants assigned to a QD dose regimen received placebo for the second dose. Participants assigned to placebo received placebo for both doses).

### Study Objectives and Endpoints

| | |
|---|---|
| Objectives | Endpoints |

Primary

| | |
|---|---|
| To assess the effect of Compound A on | Change from baseline to Week 8 in mean |
| excessive daytime sleepiness (EDS) as | sleep latency from the MWT. |
| measured by sleep latency from the | |
| Maintenance of Wakefulness Test (MWT). | |

Secondary

| | |
|---|---|
| To assess the effect of Compound A on EDS | Change from baseline to Week 8 in ESS |
| as measured by the Epworth Sleepiness Scale | total score. |
| (ESS) total score. | |
| To assess the effect of Compound A on | WCR at Week 8. |
| cataplexy as assessed by the weekly | |
| cataplexy rate (WCR). | |
| To evaluate the safety and tolerability of | Occurrence of at least 1 treatment-emergent |
| Compound A. | adverse event (TEAE). |

Additional/Exploratory

| | |
|---|---|
| To evaluate the safety and tolerability of | Occurrence of at least 1 markedly abnormal |
| Compound A. | value (MAV) for postdose laboratory values. |
| | Occurrence of at least 1 MAV for postdose |
| | vital signs. |
| | Occurrence of at least 1 MAV for postdose |
| | electrocardiogram (ECG) parameters. |
| | Time-matched change in blood pressure |
| | (BP) and heart rate (HR) from baseline to |
| | specified postdose time points. |
| | Change in ambulatory BP and HR |
| | parameters from baseline. |
| | Number (and percent) of participants with at |
| | least 1 endorsement of Item 4 or 5 on the |
| | Columbia Suicide Severity Rating Scale (C- |
| | SSRS). |
| To assess the effect of discontinuation of | Change from baseline in ESS total score, |
| Compound A, as assessed by ESS, WCR, and | WCR, and sleep parameters reported in the |
| patient-reported sleep parameters. | patient-reported sleep electronic diary (e- |
| | diary) at the follow-up visit 1 week after |
| | drug discontinuation. |
| To assess the effect of Compound A on | Change from baseline in number of lapses |
| sustained attention as measured by the | on PVT. |
| psychomotor vigilance test (PVT). | |
| To assess the effect of Compound A on overall | PGI-I and CGI-I scores. |
| narcolepsy symptoms measured by the | |
| Clinical Global Impression Scale - Global | |
| Improvement Scale (CGI-I) and the Patient | |
| Global Impression of Improvement (PGI-I) | |
| scale. | |
| To assess the effect of Compound A on | Change from baseline in NSS-CT. |
| severity of narcolepsy symptoms measured by | |
| narcolepsy Severity Scale for Clinical Trials | |
| (NSS-CT). | |
| To assess the effect of Compound A on | Change from baseline in EQ-5D-5L index |
| mobility, self-care, usual activities, | score. |
| pain/discomfort and anxiety/depression as | |
| assessed by the EuroQol-5 Dimensions 5- | |
| Levels (EQ-5D-5L) scale. | |
| To assess the effect of Compound A on quality | Change from baseline in quality of life as |
| of life of participants, as assessed by the Short | measured by SF-36 domain scores. |
| Form-36 Survey (SF-36). | |
| To assess the efficacy of Compound A on | Change from baseline in the FINI domain |
| functional impacts of narcolepsy as assessed | scores. |
| by the Functional Impacts of narcolepsy | |
| (FINI) scale. | |
| To assess the effect of Compound A on quality | Change from baseline in frequency of |
| of nocturnal sleep and presence or absence of | refreshing nocturnal sleeps, sleep paralysis, |
| rapid eye movement sleep-related | hypnagogic/hypnopompic hallucinations, |
| abnormalities, including | and nocturnal awakenings reported in the |
| hypnagogic/hypnopompic hallucination, sleep | daily e-diary. |
| paralysis, and nocturnal awakenings from the | |
| daily e-diary. | |
| To assess the effects of Compound A on | Change from baseline in CPAL, ONB, and |
| memory, working memory, and processing | iDSST-s measurements. |
| speed as measured by the cognitive tests | |
| (Continuous Paired Associate Learning | |
| [CPAL] test, One Back test [ONB], and | |
| international Digit Symbol Substitution Test- | |
| symbols [iDSST-s]). | |
| To assess the treatment satisfaction with | Treatment satisfaction as measured by the 4 |
| Compound A as measured by the Treatment | dimensions of the TSQM. |
| Satisfaction Questionnaire for Medication | |
| (TSQM). | |
| To assess the effect of Compound A on sleep | Change from baseline in various nPSG |
| architecture as measured by nocturnal | measures, including nocturnal awakenings. |
| polysomnography (nPSG). | |

### Representative Study Results

### Example 1A: Efficacy and Safety of Compound A

Endpoints included change from baseline to Week 8 in mean sleep onset latency on the Maintenance of Wakefulness Test (SOL-MWT; primary endpoint), ESS total score, weekly cataplexy rate (WCR), and occurrence of treatment-emergent adverse events (TEAEs).

A total of 112 participants, previously withdrawn from stimulant and anti-cataplectic medication, were randomized (0.5 mg/0.5 mg n=23, 2 mg/2 mg n=21, 2 mg/5 mg n=23, 7 mg n=23, placebo n=22). Participants had a mean age of 34.0 years and ESS score of 18.5 at baseline; 51.8% were female. Least squares (LS) mean (SE) changes from baseline to Week 8 in SOL-MWT were: 12.49 (2.13), 23.50 (2.04), 25.42 (2.07), 14.96 (1.95), and −1.16 (2.06) minutes with 0.5 mg/0.5 mg, 2 mg/2 mg, 2 mg/5 mg, 7 mg and placebo, respectively (LS mean difference versus placebo all P≤0.001). After adjustment for multiple comparisons, statistically significant changes from baseline to Week 8 were achieved for all dose groups versus placebo for SOL-MWT and ESS, and for the 2 mg/2 mg and 2 mg/5 mg dose groups for WCR. TEAEs occurred in 77.8% (70/90) patients on Compound A versus 31.8% (7/22) on placebo. The most common TEAEs were urinary urgency/frequency and insomnia. No treatment-related serious TEAEs or discontinuations due to TEAEs occurred during the study.

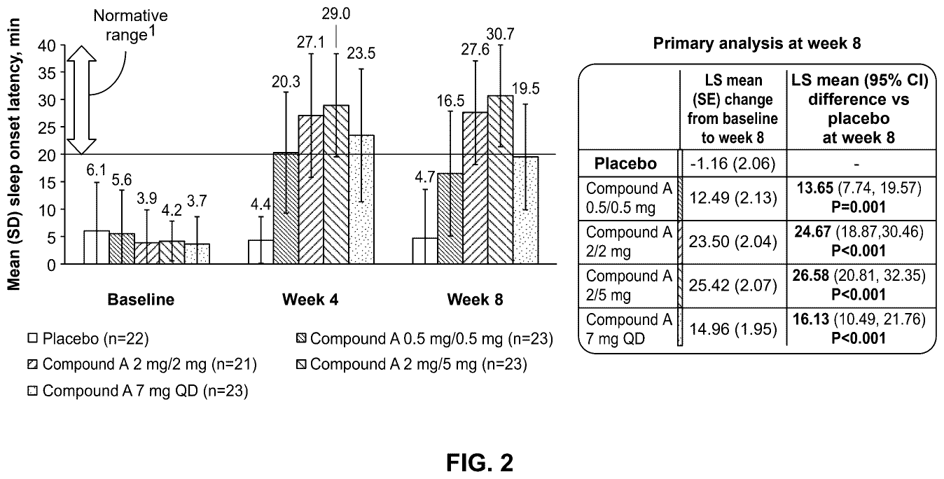

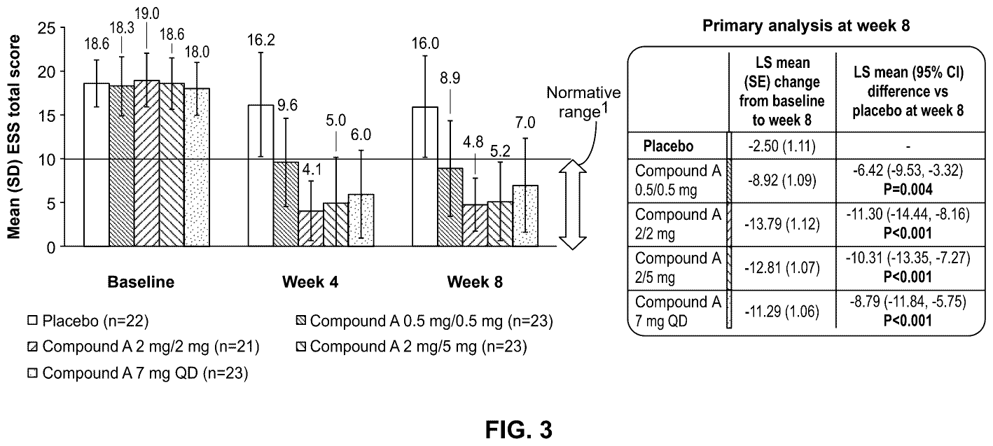

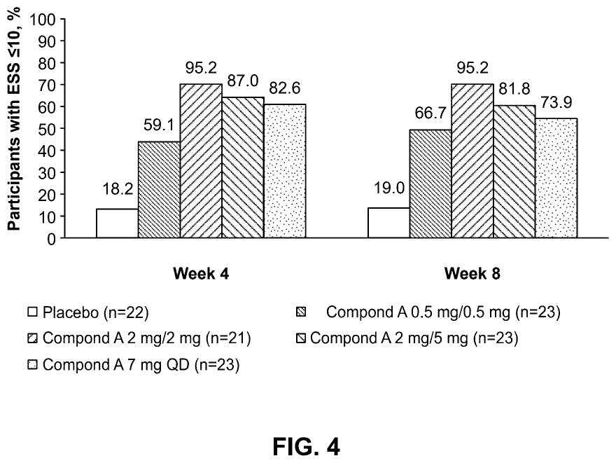

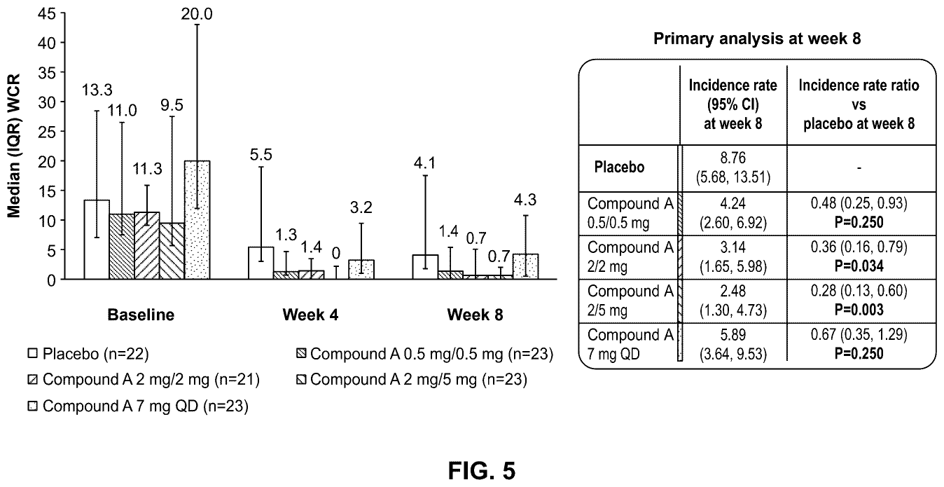

Detailed results are shown in FIGS. 2-5.

Conclusion: In this Phase 2 study, Compound A showed statistically significant and clinically meaningful improvements in objective measures of wakefulness, subjective measures of sleepiness, and in cataplexy frequency vs placebo over an 8-week treatment period. Compound A was generally well tolerated. There were no treatment-related serious TEAEs, and no discontinuations due to TEAEs. The majority of TEAEs were mild to moderate in severity and self-limiting. The most frequently reported TEAEs are in line with the on-target effects of the drug. No new safety risks were identified in relation to adverse events, vital signs, laboratory, or ECG data No cases of hepatotoxicity or visual disturbances were reported.

### Example 1B: Sleep Architecture

Polysomnography (PSG) was collected at baseline and after 27 and 54 days of treatment. Sleep was manually scored following AASM guidelines; sleep metrics were extracted then computed by whole night and by quarter-night. Continuous accelerometry (Empatica EmbracePlus watch) allowed assessment of sleep in a naturalistic manner at home. Daily sleep diaries were used to capture subjective reports. Mixed effects models were fit with treatment, quarter-night, visit, and their interactions as fixed effects and random intercepts per subject. A reference PSG dataset (Vilela M, et al. J Sleep Res 2024; e14216) from CHU Montpellier (49 clinical controls and 114 patients with NT1, all drug-free) was analyzed to establish DNS characteristics of NT1.

Compound A showed little or no effect on standard (whole night) PSG summary metrics (Table 3) in the 0.5 mg/0.5 mg and 2 mg/2 mg cohorts and few significant changes in the 2 mg/5 mg cohort. In contrast, the 7 mg QD cohort showed a worsening of sleep characteristics versus baseline.

TABLE 3
PSG Changes From Baseline to Week 8

| | | Cmpd A | Cmpd A | Cmpd A | Cmpd A |
|---|---|---|---|---|---|
| | Placebo | 0.5/0.5 mg | 2/2 mg | 2/5 mg | 7 mg QD |
| | (n = 22) | (n = 23) | (n = 21) | (n = 23) | (n = 23) |
| TST, min | −10.2 | −5.4 | 8.4 | −19.9 | −42.7* |
| SOL, min | −7.3 | 0.4 | −1.9 | −3.4 | −6.8 |
| WASO, min | −15.9 | −3.6 | −2.4 | 11.0 | 38.1* |
| % of time | −1.99 | −0.39 | −0.43 | 2.57 | 7.81* |
| in WASO | | | | | |
| % of time | 2.45* | 1.13 | 1.76 | 3.55* | 2.90* |
| in N1 | | | | | |
| % of time | −2.82 | −1.75 | 0.65 | −1.69 | −6.10* |
| in N2 | | | | | |
| % of time | 0.26 | −0.27 | 0.45 | −0.44 | −0.12 |
| in N3 | | | | | |
| % of time | 2.01 | 1.28 | −2.43 | −4.08* | −4.48* |
| in REM | | | | | |
| No. of | −1.3 | −2.3 | 2.5 | 3.2 | 6.3* |
| awakenings | | | | | |

SOL, sleep onset latency; TST, total sleep time. Estimated change from baseline to week 8 in each

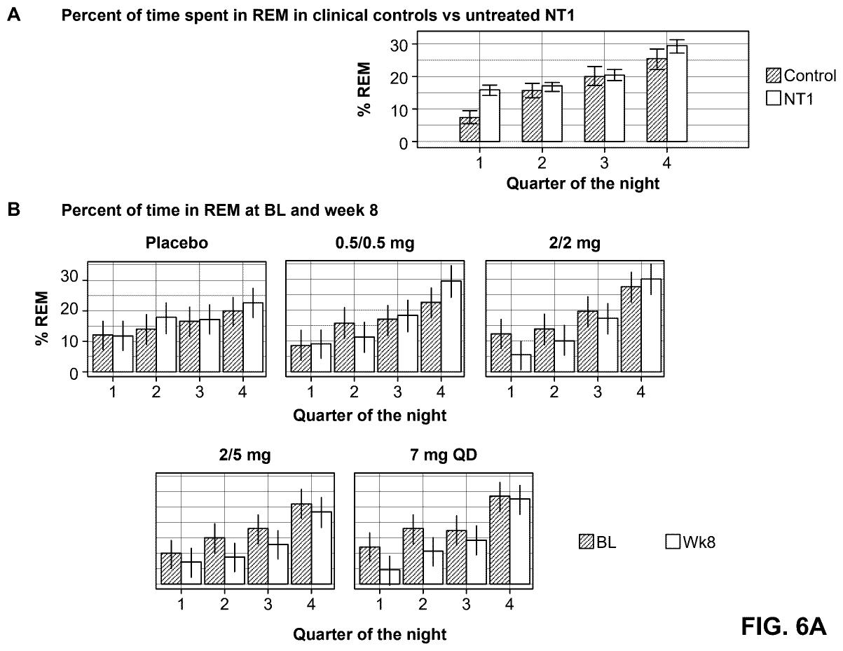

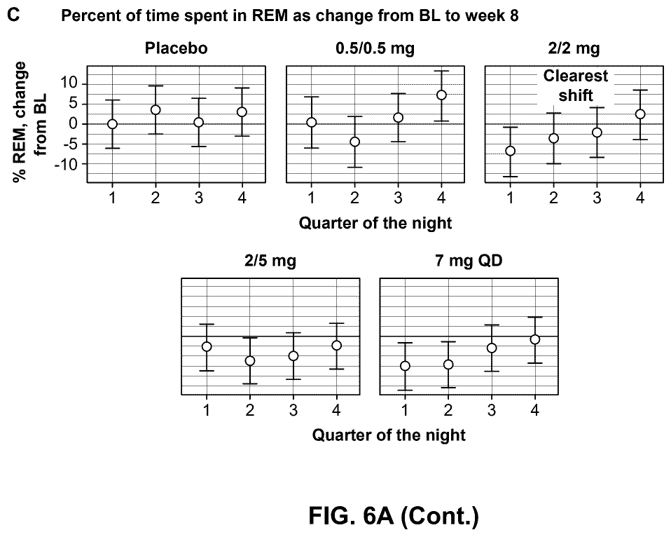

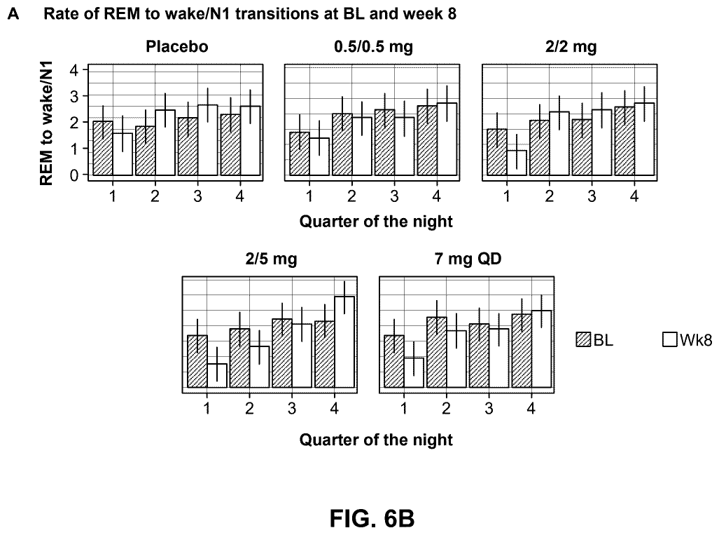

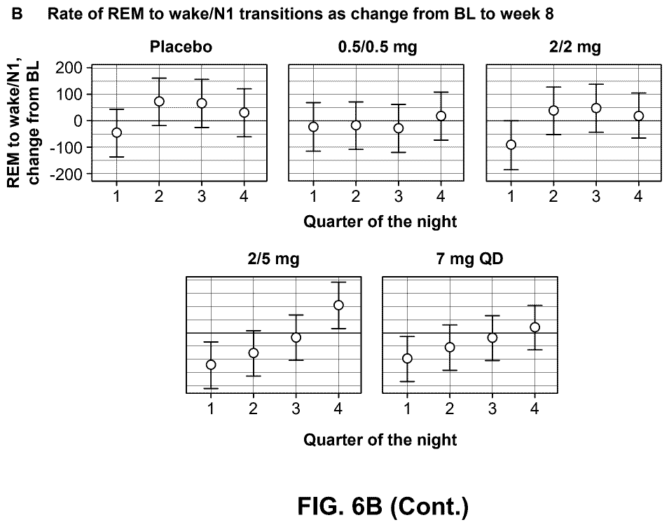

REM sleep data from in-clinic PSG are shown in FIG. 6A and FIG. 6B. FIG. 6A (Panels B and C) show Compound A shifts REM sleep architecture closer to clinical controls (Panel A). Compound A treatment shifted REM sleep from early to later in the night without reducing the total REM at week 8. This demonstrates a shift closer to clinical controls (Panel A) where REM is typically low in the first half of the night then increased in the second half of the night versus people with NT1 who spend more time in REM early in the night (Panels B and C). FIG. 6B shows Compound A reduced the rate of sleep/wake transitions early in the night between REM and wake/N1, which are common in people with NT1 (see Dauvilliers Y, et al. Sleep Med 2017; 40:53-7) except in the 0.5 mg/0.5 mg cohort.

Self-reported sleep diaries indicated that the REM changes were accompanied by significant reductions in the rate of disturbing dreams across all treated cohorts and weeks (average estimated change from baseline was −1.5 nights/week [range −1.8 to −0.8]). At baseline, participants reported disturbing dreams, which contribute to DNS, an average of 2.8 nights/week (no significant differences across cohorts) in response to “Did you have disturbing dreams, nightmares, or frightening dreams during the night?” These reductions potentially relate to improvements in REM regulation with Compound A.

In sleep diaries, 41 of 112 participants reported sleep onset insomnia in Week 1 of treatment, decreasing to 9 of 112 by Day 15.

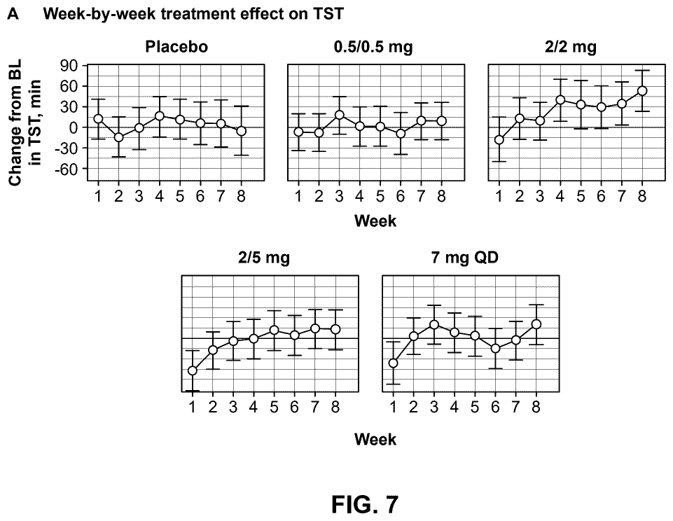

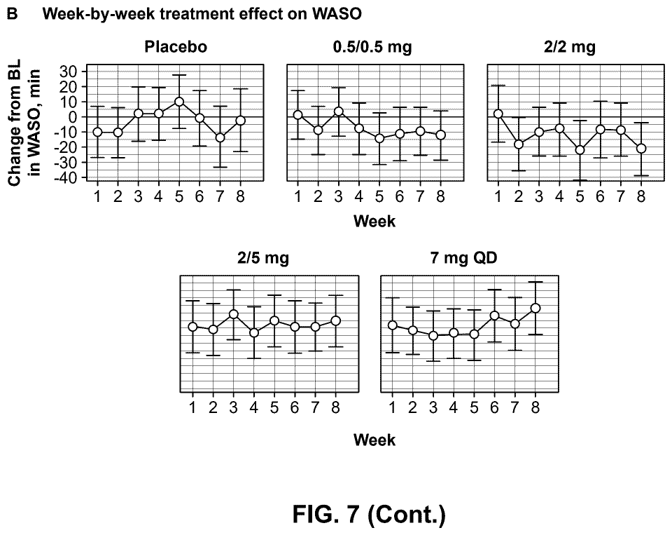

FIG. 7 shows the effects of Compound A on objective measurements of sleep using at-home continuous accelerometry. Total sleep time (TST) and wakefulness after sleep onset (WASO) measures of at-home nighttime sleep showed minimal changes overall with potential improvements in the 2 mg/2 mg cohort. During week 1 only, reduced TST was observed in the 2 mg/5 mg and 7 mg QD cohorts, resolving over time (Panel A). Consistent with sleep diaries, adverse events of mild to moderate insomnia were reported by 48% of Compound A-treated participants and most resolved within 1 week. Compound A treatment showed a trend towards decreasing WASO in the 0.5 mg/0.5 mg and 2 mg/2 mg cohorts (Panel B). Conclusion: Treatment with Compound A may help normalize REM architecture and reduce disturbing dreams without changing WASO or deep sleep.

### Example 1C: Blood Pressure Nocturnal Dipping Phenotype

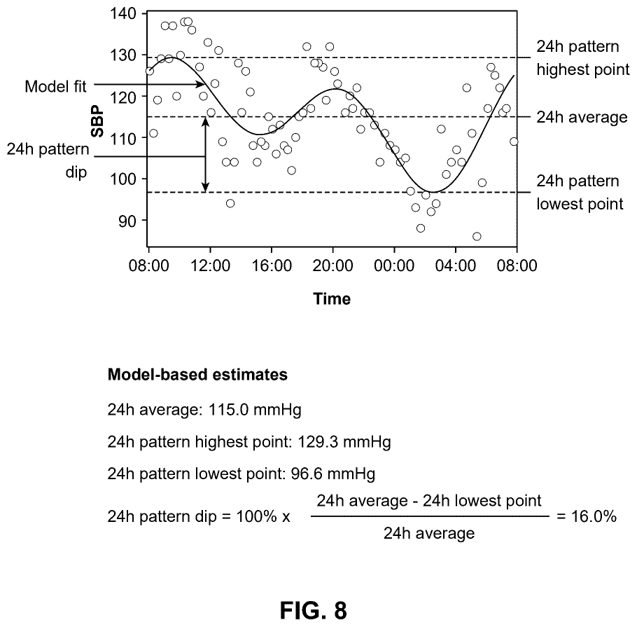

Blunted nocturnal blood pressure (BP) dip, a potential risk factor for cardiovascular disease, may affect approximately ⅓ of patients with narcolepsy type 1 (NT1). Ambulatory BP monitor (ABPM) data from 112 study patients with NT1 was measured. Using functional data analysis, systolic BP (SBP) and diastolic BP (DBP) were estimated in 24-hour patterns and key parameters were extracted, such as average, waveform amplitude, lowest and highest values, and dip amplitude (defined as [average-lowest]/average) at baseline and Week 6. FIG. 8 shows the 24-hour approach for nocturnal BP dip calculation using SBP as an example. Non-dippers were defined as having DBP 24-hour dip below a threshold corresponding to DBP daytime (6 am-10 pm) to nighttime (12 pm-6 am) drop of <10%. Longitudinal changes in BP parameters were quantified across five randomized treatment arms (placebo, Compound A 0.5 mg twice 3 hours apart, 2 mg twice 3 hours apart, 2 mg then 5 mg 3 hours later, and 7 mg once-daily) at Week 6 post-baseline. Associations between BP and sleep parameters were assessed longitudinally by matching ABPM data from baseline and Week 6 with nocturnal polysomnography data from baseline and Week 8, respectively.

At baseline, mean (SD) DBP daytime to nighttime drop was 14.7% (8.1) and DBP 24-hour pattern dip was 17.8% (6.6). In the whole population, compared with baseline, there were no significant changes in DBP 24-hour pattern dip for any Compound A dose or placebo at Week 6 (p>0.05; n=111). However, in non-dippers (n=38; 33.9%), Compound A doses of 2 mg/2 mg, 2 mg/5 mg and 7 mg showed significant DBP dip increases of 7.9% (p=0.001; n=7), 8.1% (p=0.008; n=4) and 6.6% (p=0.001; n=11) respectively, versus increases of 3.7% (p=0.076; n=8) and 2.7% (p=0.119; n=8) for placebo and Compound A 0.5 mg/0.5 mg groups, respectively. These significant DBP dip increases were concurrent with decreases in DBP 24-hour pattern lowest point. Compound A showed no significant changes in DBP 24-hour average or DBP 24-hour pattern highest point. There were no significant changes in SBP or DBP dip among baseline DBP dippers (n=73) in either treatment arm. Results are shown in Table 4.

TABLE 4
BP Characteristic Changes at Week 6 From
Baseline, in Baseline DBP Non-Dippers
LS Mean (P-value) Change at Week 6 from Baseline
SBP

| | | | | | |
|---|---|---|---|---|---|
| 24-h average | −2.72 | −1.42 | 0.05 | −4.13 | 1.32 |
| | (0.359) | (0.631) | (0.987) | (0.325) | (0.601) |
| 24-h pattern highest | 0.64 | −0.87 | 1.45 | −1.86 | 3.31 |
| | (0.839) | (0.785) | (0.689) | (0.678) | (0.226) |
| 24-h pattern lowest | −4.11 | −2.48 | −6.55 | −8.80 | −2.44 |
| | (0.306) | (0.535) | (0.152) | (0.125) | (0.474) |
| 24-h pattern dip, % | 1.49 | 1.20 | 5.36 | 4.11 | 3.28 |
| | (0.458) | (0.550) | (0.022) | (0.152) | (0.061) |

DBP

| | | | | | |
|---|---|---|---|---|---|
| 24-h average | −1.58 | 0.17 | −0.08 | −3.40 | −0.75 |
| | (0.330) | (0.918) | (0.967) | (0.144) | (0.584) |
| 24-h pattern highest | 1.61 | 0.75 | 1.62 | −1.18 | 1.26 |
| | (0.481) | (0.743) | (0.536) | (0.714) | (0.518) |
| 24-h pattern lowest | −3.79 | −1.73 | −5.93 | −8.71 | −5.13 |
| | (0.063) | (0.385) | (0.012) | (0.004) | (0.004) |
| 24-h pattern dip, % | 3.74 | 2.67 | 7.91 | 8.11 | 6.61 |
| | (0.076) | (0.200) | (0.001) | (0.008) | (0.001) |

LS, least squares; QD, once daily.
Statistically significant results (P-value < 0.05) are highlighted.

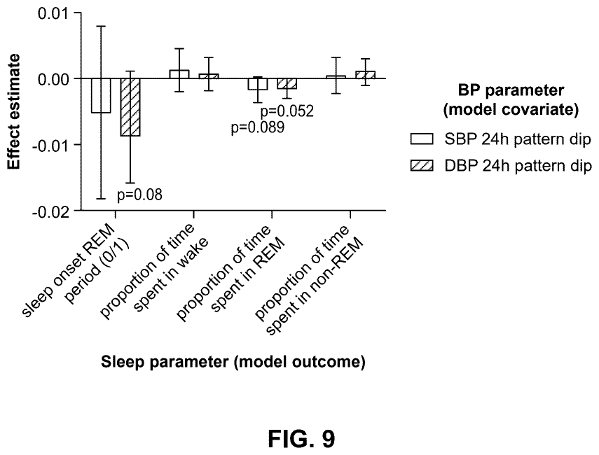

Longitudinally, higher DBP 24-hour pattern dip was directionally associated with decreased REM (p=0.052; n=112) and decreased probability of sleep-onset REM period occurrence (p=0.080; n=112). Overall, similar observations were made for SBP. Results are shown in FIG. 9.

Conclusion: In patients with NT1, Compound A treatment showed statistically significant improvements in restoring blunted nocturnal BP dip at the 3 (out of 4) higher doses tested.

### Example 1D: Dissociation of EDS and Attentional Impairment

Cognitive impairment, particularly in attentional function, is a common and disruptive symptom of NT1, but the extent to which cognitive impairment is dependent on excessive daytime sleepiness (EDS) is unclear.

Outcomes of this study included a performance-based test of attention, the Psychomotor Vigilance Task (PVT; administered 1 and 7 hours post first dose, assessed at baseline and weeks 4 and 8) and two assessments of EDS: the Maintenance of Wakefulness Test (MWT; administered 2, 4, 6, and 8 hours post first dose, assessed over 4 sessions) and the Epworth Sleepiness Scale (ESS; using a 7-day recall period, assessed at baseline and weeks 2, 4, and 8). At baseline, associations between attention (as measured by number of PVT lapses), and objective (MWT) and subjective (ESS) measures of EDS were computed. For PVT and MWT, covariation in change throughout the day at end of study was investigated. The effect of different Compound A doses on wakefulness and attention were compared for each outcome.

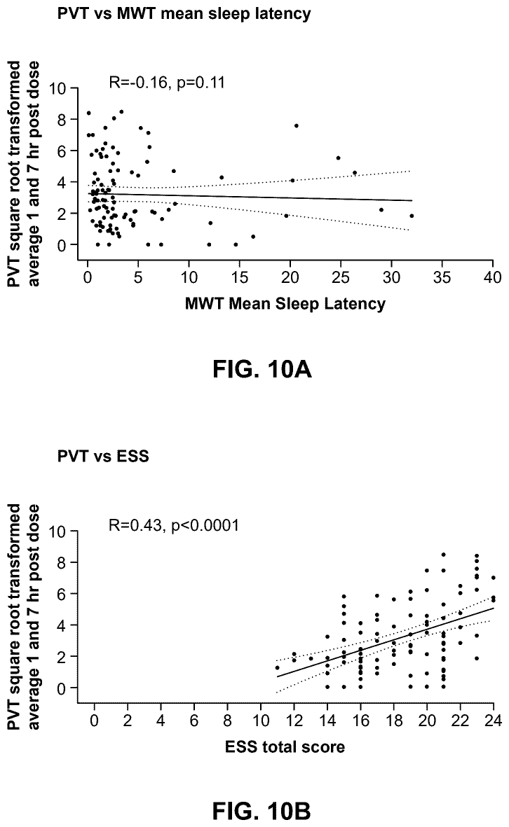

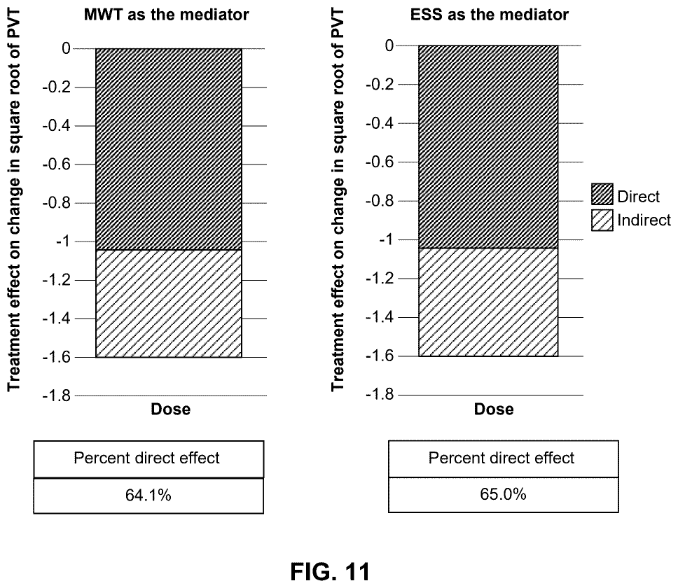

FIG. 10 shows baseline association of number of PVT lapses with MWT (Panel A) and ESS (Panel B). At baseline, PVT-lapses were not associated with MWT sleep latency but associated moderately with ESS total score. Following treatment with Compound A, PVT-lapses remained stable across the day whereas MWT sleep latency declined. Maximum benefit on PVT-lapses was achieved with doses from 0.5 mg Compound A, whereas maximum benefits on the MWT and EDS were achieved with doses of 2 mg and 2 mg/5 mg Compound A. At week 8, Compound A improved attention across all doses (all p<0.05) and remained significant for 0.5 mg after including change from baseline in EDS (defined using MWT or ESS) as a covariate. As shown in FIG. 11 a substantial portion of the effect of Compound A on improvement in PVT lapses was directly attributed to Compound A treatment (i.e., not mediated by MWT or ESS). Conclusion: These results suggest that attentional impairment can be dissociated from EDS, and both were improved by Compound A. This indicates impairment in attention may arise directly from disease biology and indirectly from sleepiness.

### Example 1E: Change in Sustained Attention, Global Functioning, Disease Severity, and Activities of Daily Living

This study investigated relationships between sustained attention and clinician's and patient's ratings of global function, disease severity, and instrumental activities of daily living (iADLs) in patients with NT1. The endpoint for PVT performance was number of lapses of attention (PVT-lapses). Relationships between a measure of sustained attention (number of PVT-lapses), measures of disease severity (Narcolepsy Severity Scale, NSS-CT), global impression of disease severity rated by clinicians (CGI-I) and patients (PGI-I), and iADLs (EQ-5D-5L Usual Activities; Functional Impacts of Narcolepsy Instrument [FINI] social activities [SA], everyday activities [EA] and everyday responsibilities [ER] domains) were assessed cross-sectionally at Week 8 and as changes from baseline to Week 8. For CGI-I, PGI-I, NSS-CT, and eQ-5D-5L Usual Activities, responses were defined as an increase (improved) or decrease (worsened) by ≥1 category or no change. For FINI domains, responders were defined as a decrease of ≥30 (FINI Everyday Responsibilities) or ≥40 (Social Activities and Everyday Activities) Points. Spearman correlations were calculated to assess the association of observed PVT lapses versus observed NSS-CT total score, and FINI category scores; a t test was performed for EQ-5D-5L Usual Activities at week 8. Longitudinal relationships between change from baseline in PVT lapses and changes in NSS-CT, EQ-5D-5L Usual Activities and 3 FINI domains over 8 weeks of treatment in a phase 2 trial with Compound A were assessed. The t tests/analyses of variance were performed to assess differences in the change from baseline in PVT lapses among the change from baseline categories in PGI-I, CGI-I, NSS-CT total score, FINI domain scores and EQ-5D-5L. PVT lapses were square-root transformed and averaged across 1 h and 7 h post morning doses for all analyses.

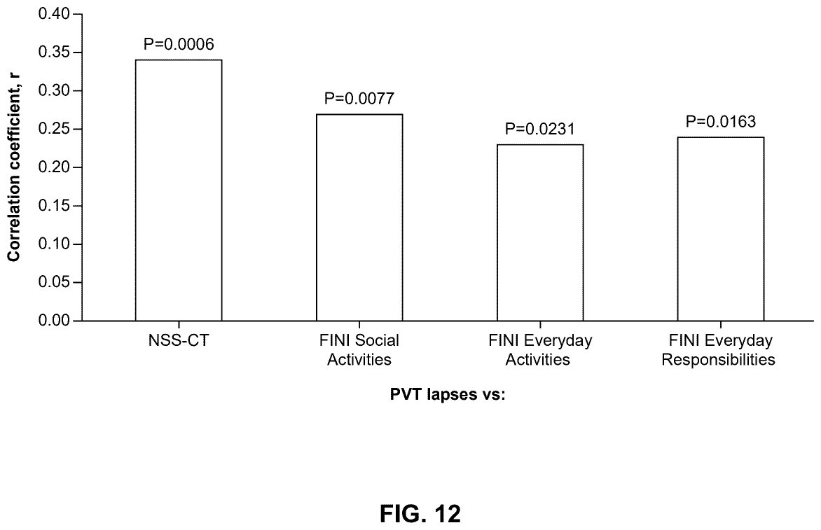

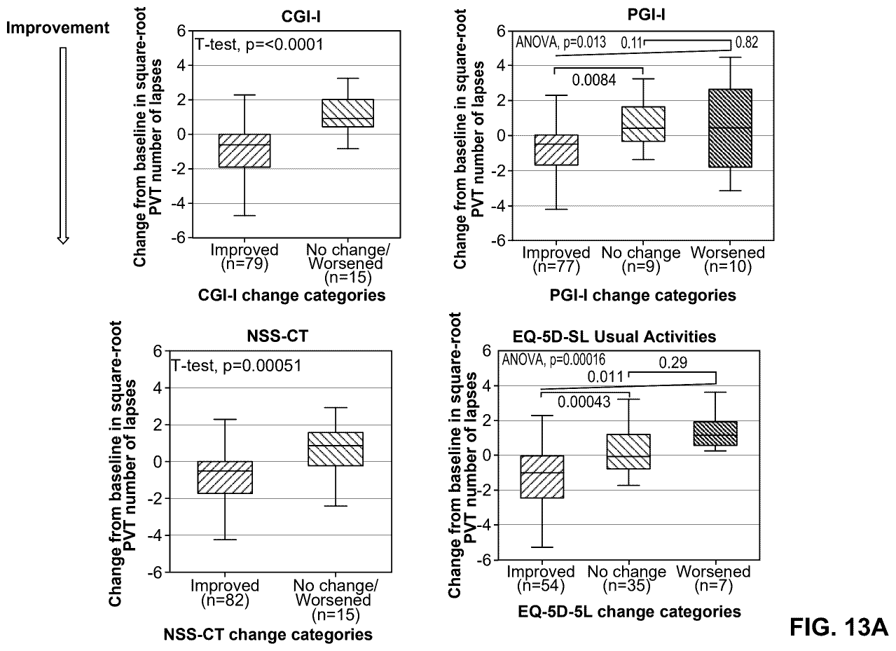

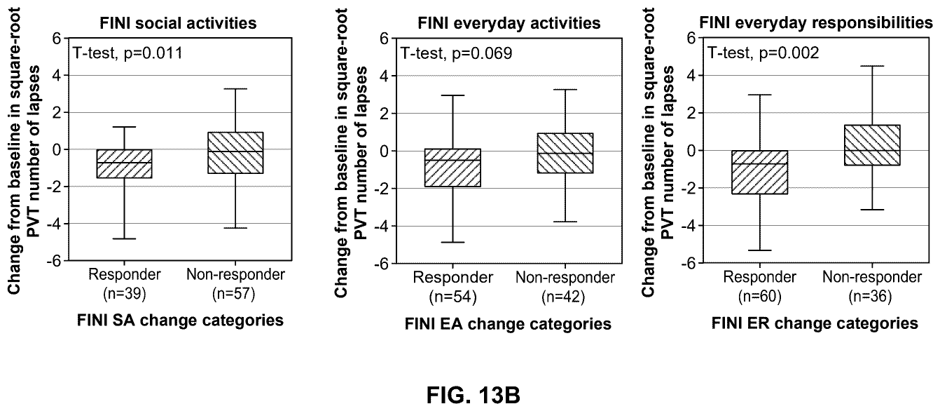

Efficacy assessments for the overall pooled study population at baseline and week 8, irrespective of treatment arm, are shown in Table 5. As shown in FIG. 12, at Week 8, greater numbers of PVT-lapses were associated with poorer scores on the NSS-CT (p=0.0007) and the three FINI domains (SA: p=0.0057, EA: p=0.0035, ER: p=0.0099). There was no significant association between PVT lapses and EQ-5D-5L Usual Activities (P=017). As shown in FIGS. 13A and 13B, improvement on PVT-lapses from baseline to Week 8 was associated with improved scores on the CGI-I (p<0.0001) and PGI-I (p=0.0095), NSS-CT (p=0.0001), EQ-5D-5L Usual Activities (p=0.0002), and the three FINI domains (SA: p=0.0037, EA: p=0.0095, ER: p=0.0013).

TABLE 5
Efficacy Assessments, Overall Population

| | |
|---|---|
| Baseline | Week 8 |
| (n = 112) | (n = 109) |

PVT lapses, mean (SD)

| | | | | |
|---|---|---|---|---|
| Morning | 14.0 | (17.1)† | 10.7 | (17.0) |
| Afternoon | 16.3 | (19.5)† | 10.2 | (15.9) |
| NSS-CT total score, mean (SD) | 30.7 | (8.9) | 13.9 | (10.8) |

FINI domain score, mean (SD)

| | | | | |
|---|---|---|---|---|
| Social Activities | 51.0 | (26.6) | 20.7 | (24.7) |
| Everyday Activities | 73.7 | (16.6) | 32.5 | (27.1) |
| Everyday Responsibilities | 60.8 | (27.0) | 24.6 | (30.8) |

EQ-5D-5L, n (%)

| | | | | |
|---|---|---|---|---|
| Usual Activities, any | 75 | (67.0) | 37 | (34.3) |
| problems (levels 2-5) | | | | |

*Pooled data for all trial participants, irrespective of treatment arm.
†Baseline morning/afternoon assessments are time matched to post-baseline assessments at 1 and 7 hours post first daily dose.

Conclusion: Cross-sectional analyses revealed that impairment in sustained attention was associated with greater disease severity and poorer iADLs in patients with NT1. Improvements in sustained attention from baseline to Week 8 were associated with improvements in global function, disease severity, and iADLs. These findings suggest changes on the PVT reflect changes in real-world attention and well-being in NT1.

### Example 1F: Treatment Satisfaction With Compound A

Cognitive impairment, a common and disruptive symptom of NT1, is characterized by deficits in attention, memory, and executive functions. The effect of Compound A on these cognitive domains in patients with NT1 was evaluated.

Post-hoc analyses of the following neuropsychological tests were conducted: the 10-minute Psychomotor Vigilance Task (PVT), the Continuous Paired Associate Learning Test (CPAL), One Back Test (ONB), and the international Digit Symbol Substitution Test-symbols version (iDSST-s). The tests were administered at baseline, Week 4, and Week 8, 1-hour post-first dose. The PVT was re-administered 7-hours post-first dose to assess the maintenance of observed performance improvements across the day.

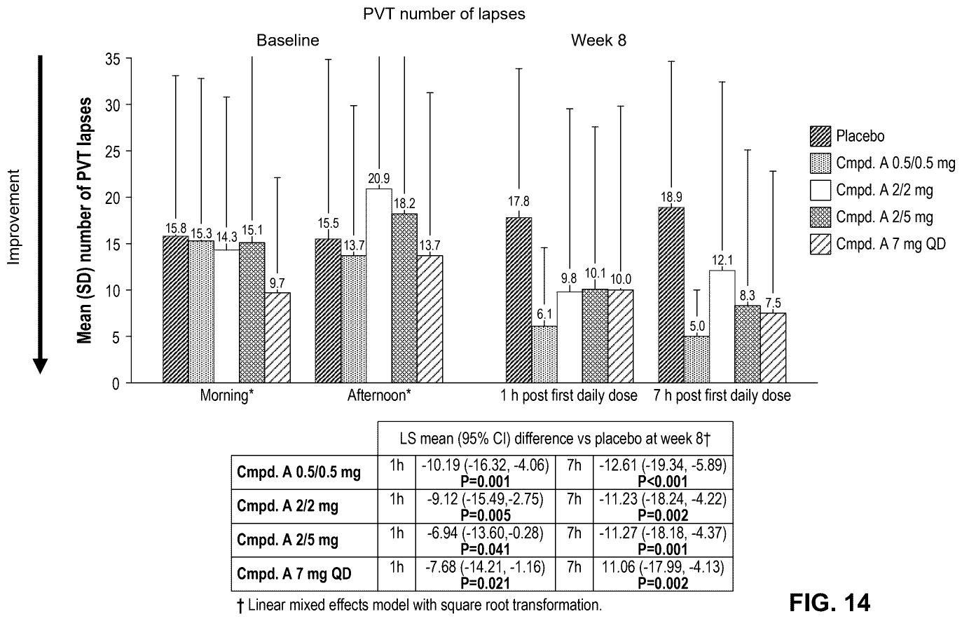

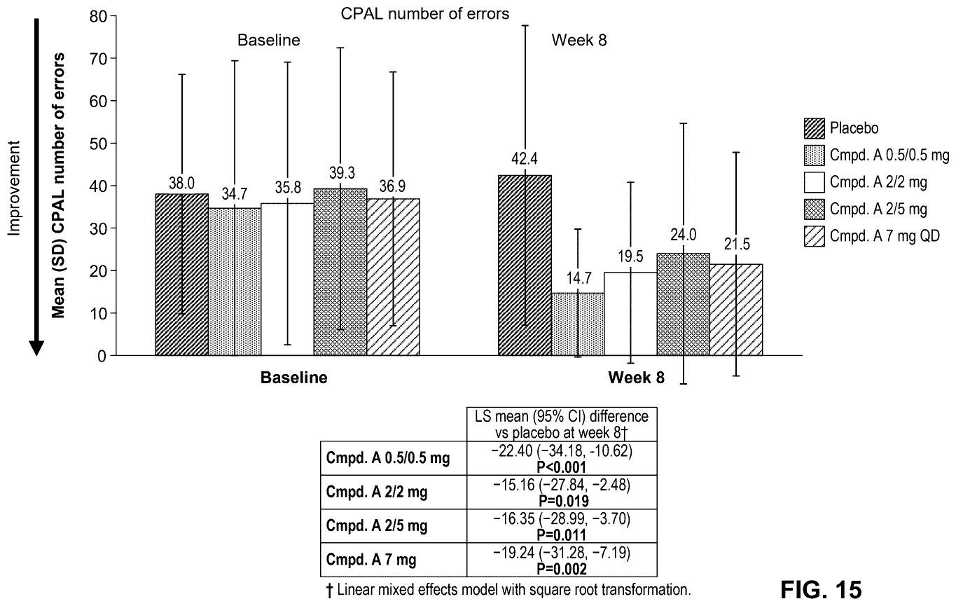

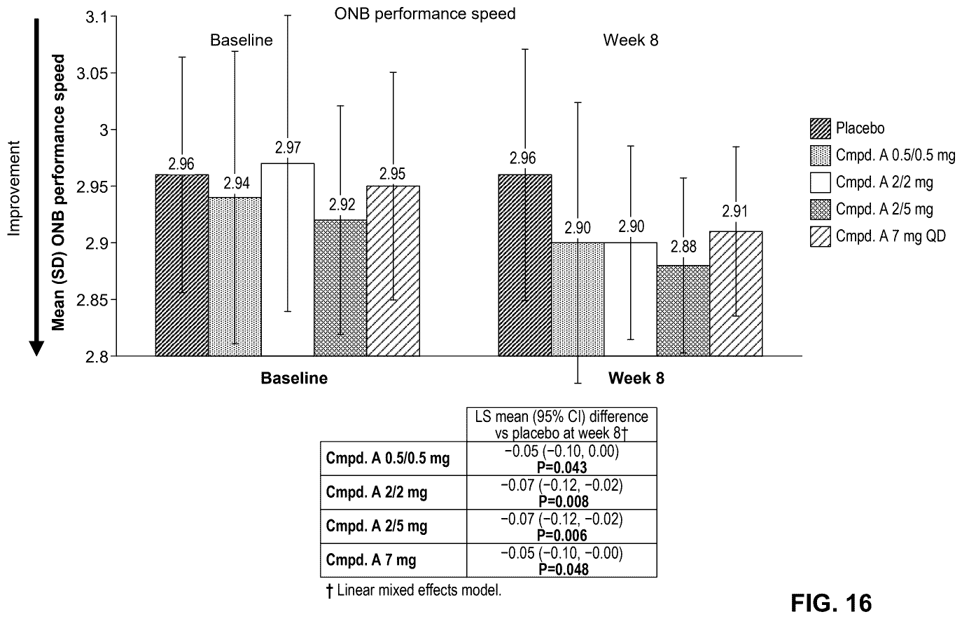

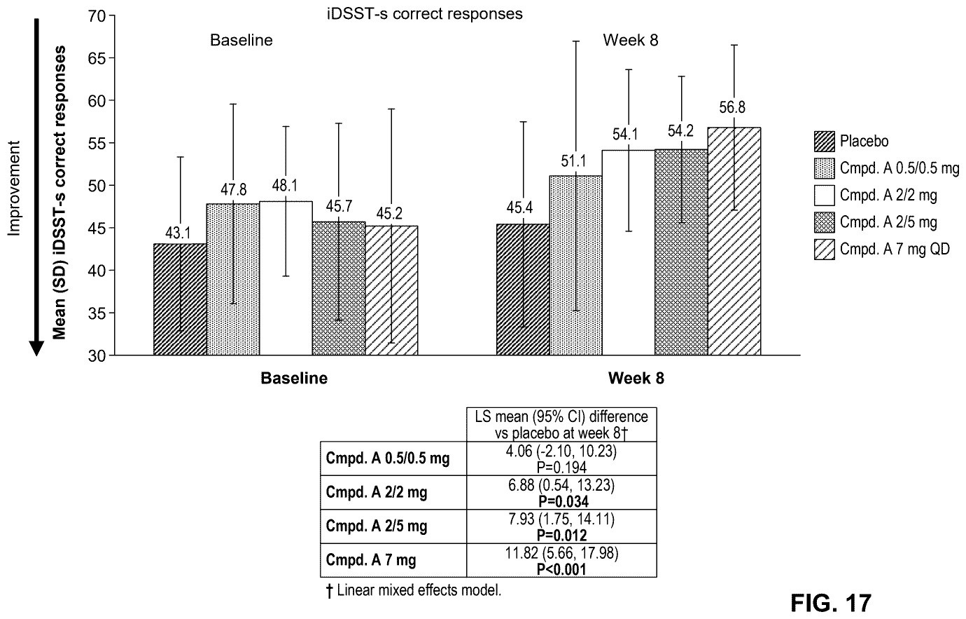

A total of 112 patients were randomized to placebo (n=22) or Compound A (0.5 mg twice 3 hours apart, n=23; 2 mg twice 3 hours apart, n=21; 2 mg then 5 mg 3 hours later, n=23; 7 mg once-daily [QD], n=23); 109 patients completed the study. For PVT lapses, least squares mean differences from placebo were significant (p<0.05) at all Compound A doses at 1 hour and 7 hours post-first doses (FIG. 14). Improvements on CPAL total errors, ONB reaction time, and iDSST-s number correct were also statistically significant across all doses (FIGS. 15, 16, 17, respectively).

Conclusion: All assessed doses of Compound resulted in improvements in sustained attention (PVT), memory (CPAL), and executive functions (ONB, iDSST) suggesting potential use for OX2R agonists in the treatment of cognitive impairment associated with NT1.

### Example 1G: Treatment Satisfaction with Compound A

Treatment Satisfaction Questionnaire for Medication (TSQM) and exit interview results from the study were evaluated to explore the impact of Compound A treatment on individuals with ICSD-3-confirmed NT1. Participants aged 18-70 years, with an Epworth Sleepiness Scale (ESS) score>12, and ≥4 partial/complete episodes of cataplexy/week were included in the trial. Participants were randomized to oral Compound A (0.5 mg twice 3 hours apart, 2 mg twice 3 hours apart, 2 mg then 5 mg 3 hours later, or 7 mg once daily), or placebo.

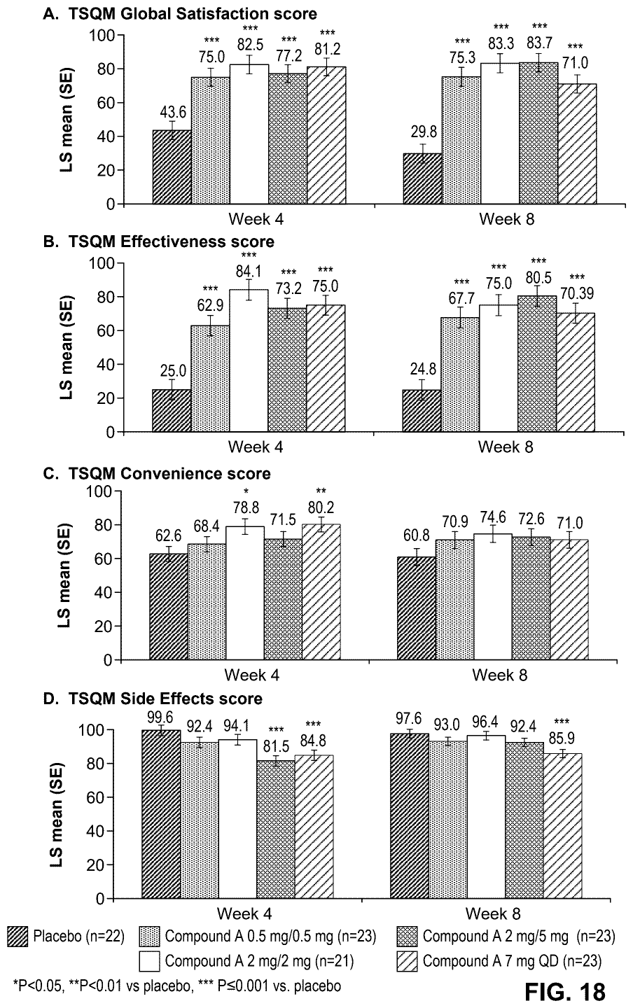

FIG. 18 shows TSQM domain scores for Global Satisfaction (Graph A), Effectiveness (Graph B), Convenience of treatment with Compound A (Graph C) and impacts of Side Effects (Graph D). At Week 8, least square (LS) mean TSQM effectiveness and global satisfaction domain scores were 67.7-80.5 versus 24.8 and 71.0-83.7 versus 29.8 across Compound A doses versus placebo, respectively (all P<0.001). Convenience domain scores for Compound A were similar to placebo (70.9-74.6 vs 60.8). The level of satisfaction with side-effects was high across Compound A treatment arms (92.4-96.4) and somewhat lower with the 7 mg-dose (85.9).

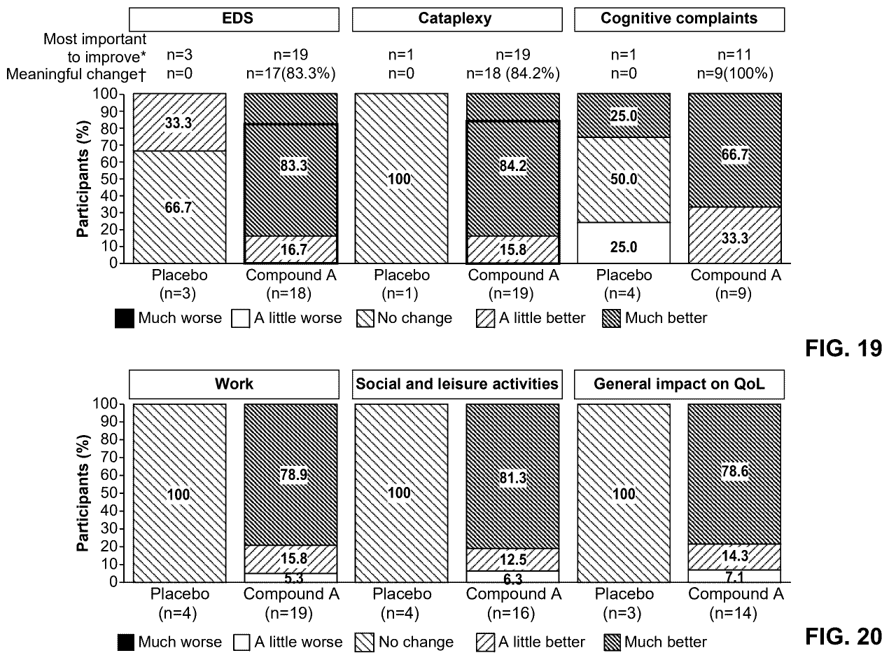

FIG. 19 and FIG. 20 show the results of exit interview outcomes at Week 8. After Week 8, 25 individuals (Compound A: n=21/placebo: n=4) participated in the exit interviews, which revealed meaningful improvements in narcolepsy symptoms and functional impacts for most Compound A participants. Symptoms rated as most important to improve from baseline were EDS, cataplexy and impaired cognitive function (FIG. 19); most frequently cited impacts were work/school-related, social/leisure activities and general quality of life (FIG. 20). Changes in impacts were meaningful for Compound A-treated participants but not for placebo-treated participants. Overall, 94.7% of Compound A-treated participants (18/19 respondents) would be willing to recommend Compound A to others with narcolepsy.

Conclusion: Quantitative (TSQM) and qualitative (exit interviews) Phase 2 data revealed high treatment satisfaction with Compound A in participants with NT1, demonstrating meaningful improvements in symptoms and functional impacts important to patients.

### Example 1H: Effect of Compound A on Function and Health-Related Quality of Life

An analysis of exploratory data from the trial investigated the effect of Compound A on functioning and HRQoL in individuals with ICSD-3 confirmed NT1. Participants aged 18-70 years (Japan: 16-70), with an Epworth Sleepiness Scale (ESS) score>12, and ≥4 partial/complete episodes of cataplexy/week were eligible for inclusion. Participants were randomized to oral Compound A (0.5 mg twice 3 hours apart, 2 mg twice 3 hours apart, 2 mg then 5 mg 3 hours later, or 7 mg once-daily), or placebo. Exploratory endpoints included 3 domains (social activities, everyday activities, and everyday responsibilities) from the 6-domain Functional Impacts of Narcolepsy Instrument (FINI) in which each item uses 5-point Likert scale and higher standardized domain scores (0-100) indicate more severe impact; the 36-item short-form (SF-36); and the EuroQoL 5-Dimensions 5-Levels (EQ-5D-5L) questionnaire.

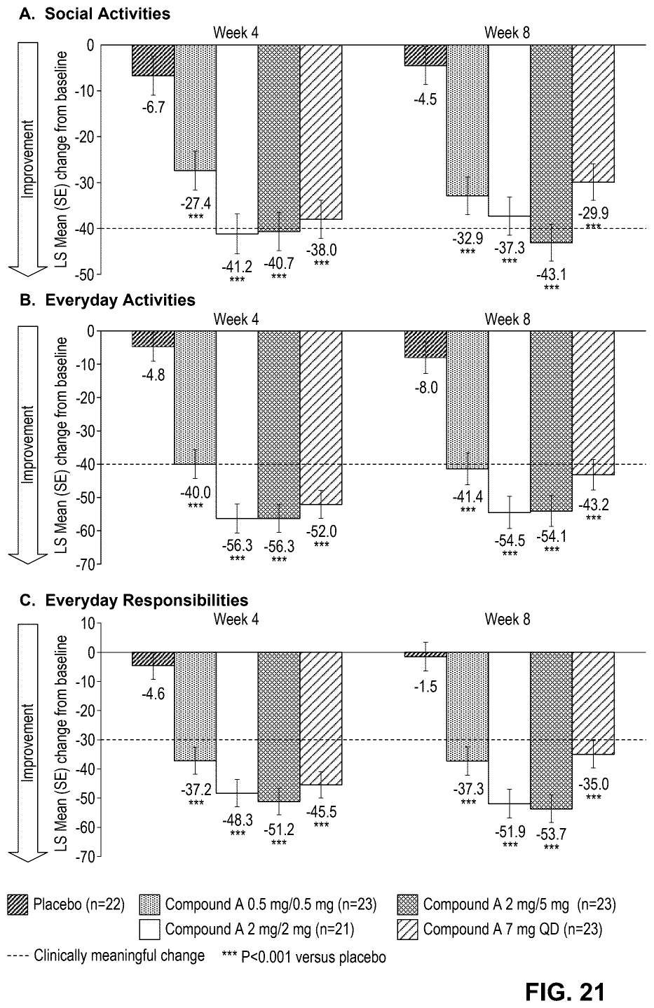

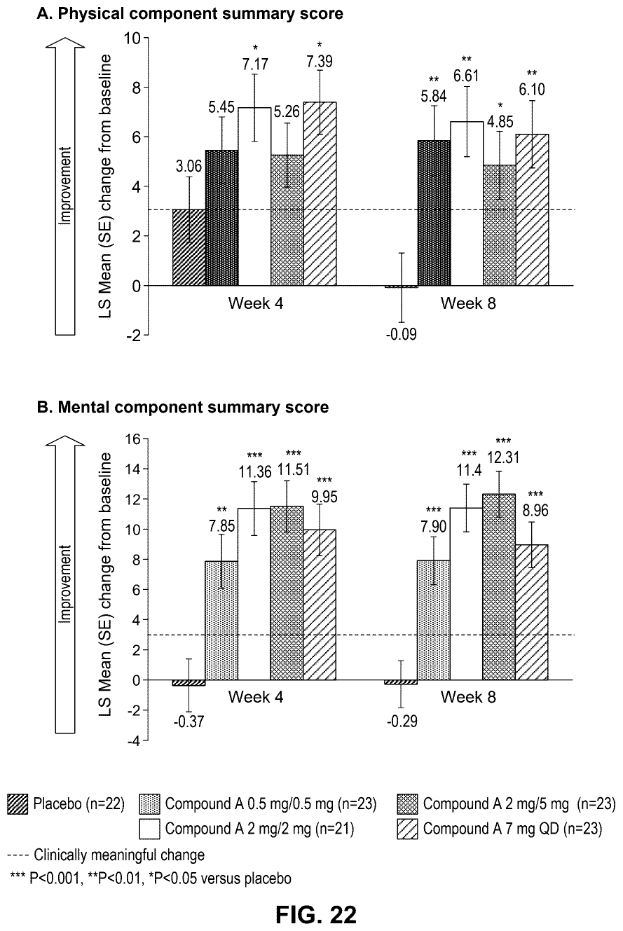

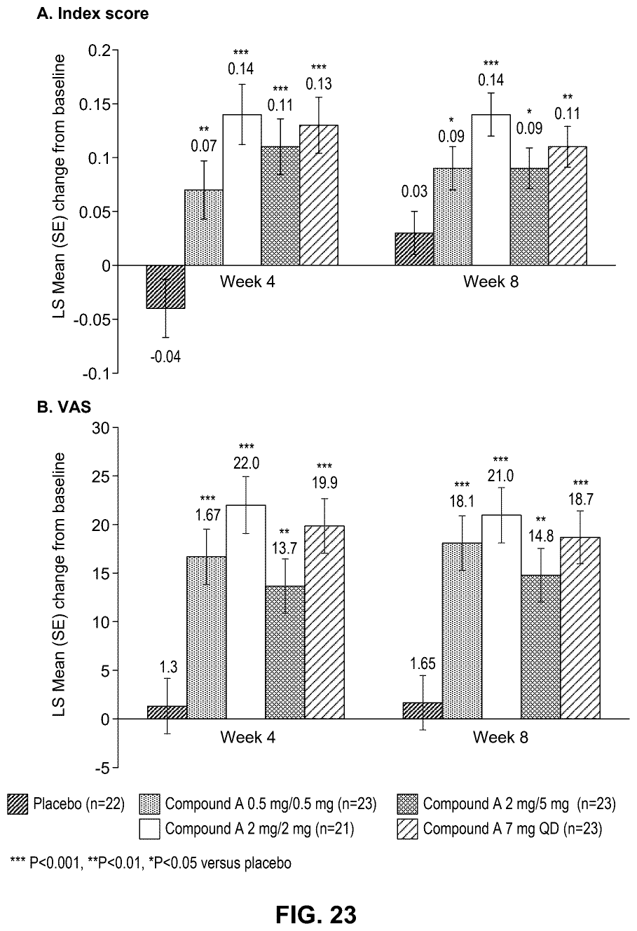

A total of 112 participants (mean age 34.0 years, and ESS score 18.5, 51.8% female) were randomized to Compound A (0.5 mg/0.5 mg n=23, 2 mg/2 mg n=21, 2 mg/5 mg n=23, 7 mg n=23) or placebo (n=22). As shown in FIG. 21, statistically significant improvements in FINI domains were achieved with Compound A at Week 8. Least square [LS] mean [SE] changes from baseline to Week 8 for Compound A doses versus placebo were −29.9 to −43.1 versus −4.5, respectively, for FINI-Social Activities (Graph A), −41.4 to −54.5 versus −8.0 for FINI-Everyday Activities (Graph B), and −35.0 to −53.7 versus-1.5 for FINI-Everyday Responsibilities (Graph C) (all p<0.001 for all doses versus placebo). Statistically significant improvements in SF-36 (FIG. 22) and EQ-5D (FIG. 23) were achieved with all Compound A doses. Least square mean (SE) changes from baseline were statistically significant for all Compound A dose groups versus placebo at weeks 4 and 8 for the Physical Component summary score (Graph A) and at week 8 for the Mental Component summary score (Graph B). LS mean (SE) changes from baseline in EQ-5D-5L Index Score (Graph A) and Visual Analog Scale (VAS, Graph B) were statistically significant across all Compound A dose groups versus placebo at weeks 4 and 8. Compound A was generally well tolerated and there were no treatment-related serious TEAEs or discontinuations due to TEAEs during the study.

Conclusion: In this Phase 2 study, Compound A significantly improved narcolepsy-specific functioning (FINI social and everyday activities, and everyday responsibilities), and general HRQoL measures assessing mental and physical health (SF-36), and overall health status (EQ-5D-5L) in people with NT1 over 8 weeks.

### Example 1I: Effect of Compound A on the Severity of Symptoms

Methods: An analysis of exploratory data from the study was conducted to explore disease severity in individuals with ICSD-3 confirmed NT1. Eligible participants were age 18-70 years (Japan: 16-70), with an Epworth Sleepiness Scale (ESS) score>12, and >4 partial/complete episodes of cataplexy/week. Exploratory endpoints included change from baseline to Week 8 in the narcolepsy Severity Scale for clinical trials (NSS-CT), and Clinical Global Impression (CGI) and Patient Global Impression (PGI) at Week 8. The NSS-CT, a validated, self-administered 15-item scale, evaluates the severity, frequency, and impact of five main narcolepsy symptoms (higher scores indicate more severe symptoms).

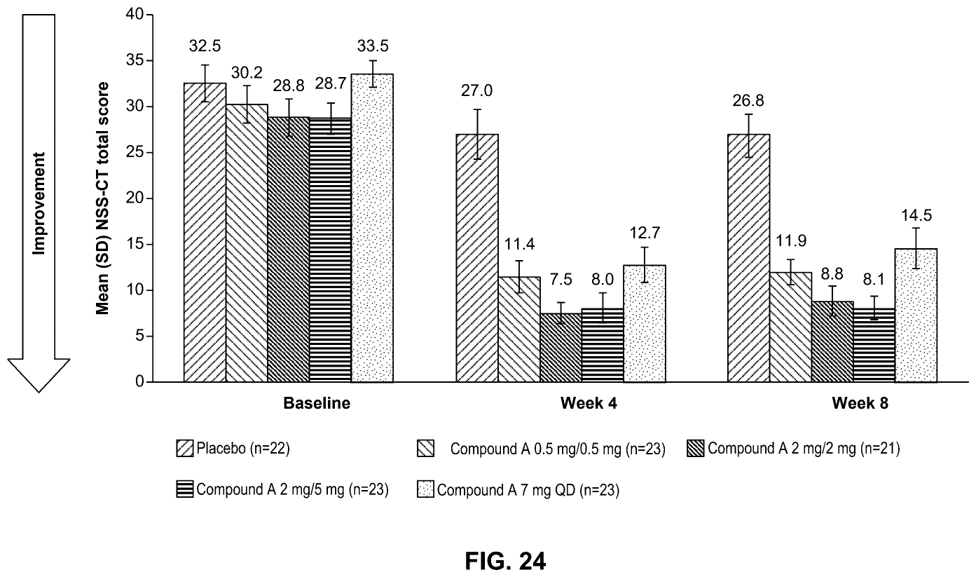

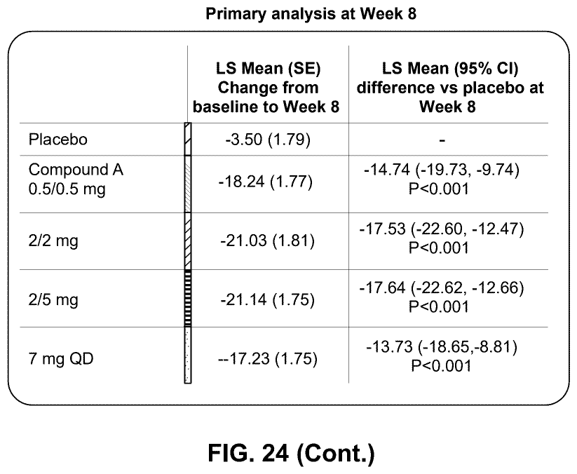

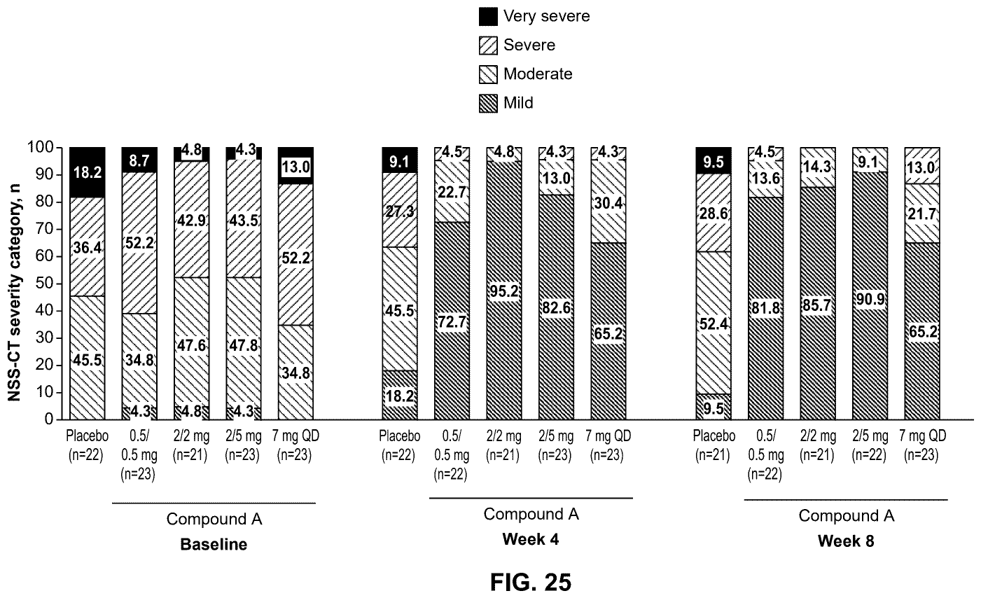

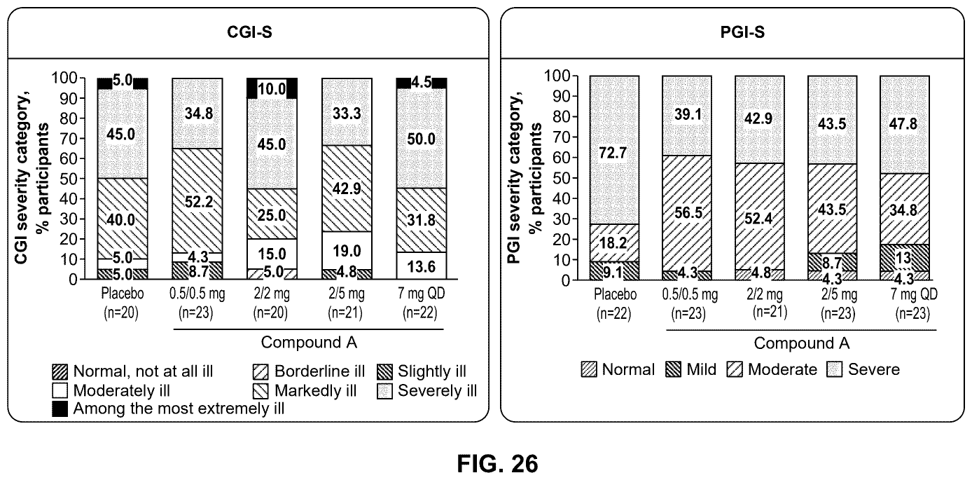

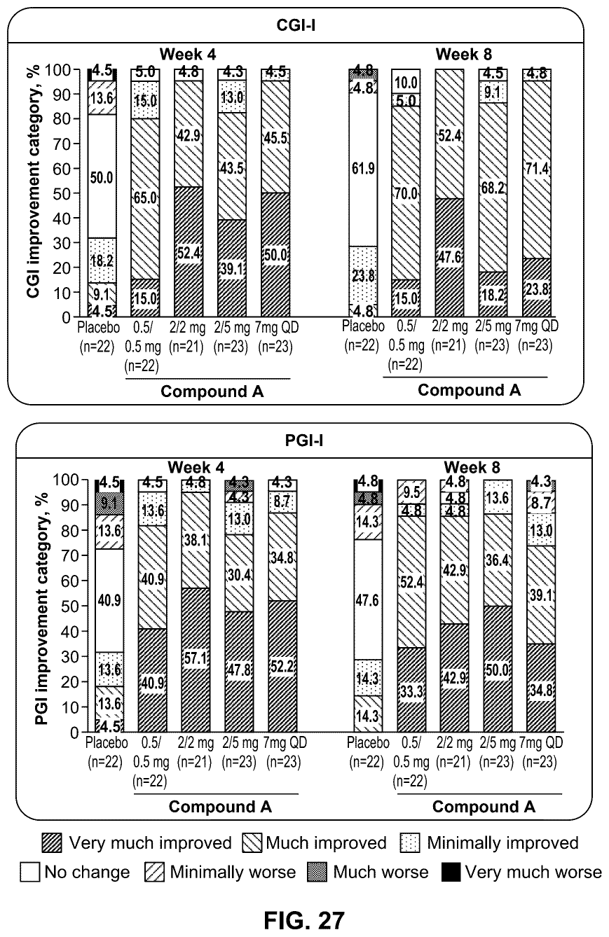

A total of 112 participants (mean age of 34.0 years, 51.8% female, ESS score 18.5) were randomized to Compound A (0.5 mg/0.5 mg n=23, 2 mg/2 mg n=21, 2 mg/5 mg n=23, 7 mg n=23) or placebo (n=22). As shown in FIG. 24, significant improvements in NSS-CT total score were achieved with Compound A (Least square [LS] mean [SE] change from baseline to Week 8: −18.2 [0.5 mg/0.5 mg], −21.0 [2 mg/2 mg], −21.1 [2 mg/5 mg], −17.2 [7 mg]) with Compound A and −3.5 for placebo; all P<0.001 vs placebo). In addition, as can be seen in FIG. 25, more participants on Compound A reported mild or moderate disease on NSS-CT compared with placebo. Assessments of disease severity at baseline were similar between treatment groups (FIG. 26), and as shown in FIG. 27, at Week 8, >70% of patients treated with Compound A were much/very much improved on the PGI and CGI, consistent with NSS-CT. Compound A was generally well tolerated and there were no treatment-related serious TEAEs or discontinuations due to TEAEs during the study.

Conclusion: In this Phase 2 study, Compound A significantly improved both physician-(CGI) and participant-reported (PGI, NSS-CT) measures of overall treatment experience and disease severity across the spectrum of narcolepsy symptoms in participants with NT1 over 8 weeks.

### Example 2. A Randomized, Double-Blind, Placebo-Controlled Study to Evaluate the Efficacy, Safety, and Tolerability of Compound A for the Treatment of Narcolepsy Without Cataplexy (Narcolepsy Type 2)

The study was performed in approximately 60 (male and female) participants with NT2, who were randomized such that each participant had an equal chance of being assigned to any 1 of 3 treatment arms: 2 Compound A dose regimens, or matching placebo. Randomization was stratified by region. Starting on the morning of Day 1, the study drug was administered at approximately the same time each day for 8 weeks.

Participants who provided informed consent completed a screening period of up to 45 days to washout any NT2 medication (if applicable). Participants were asked to complete an e-diary, starting from the initial screening visit, no later than Day-16.

Participants remained confined overnight at the study site during the following times:
Days −2 to 1 (1 mandatory overnight at Day −2; 1 optional overnight at Day-1)
Days 27 to 28 (1 overnight)
Days 55 to 56 (1 overnight)

After the Week 8 visit, participants had the option to participate in a long-term extension (LTE) study under a separate protocol (Example 3), assuming the protocol was open for enrollment. Participants who enrolled in the LTE study did not have follow-up visits in this study.

For participants who did not participate in the LTE study, every effort was deployed to have them complete a first follow-up visit approximately 7 days after the final study drug intake and a second follow-up visit (home healthcare visit) approximately 28 days after the final study drug intake. For participants who terminated the study early, every effort was deployed to have them complete an early termination visit as soon as possible and a follow-up visit (in-clinic visit or home healthcare, if available) approximately 28 days after the last dose of study drug. Participants not participating in the LTE study could restart their discontinued medications after the first follow-up visit or early termination visit.

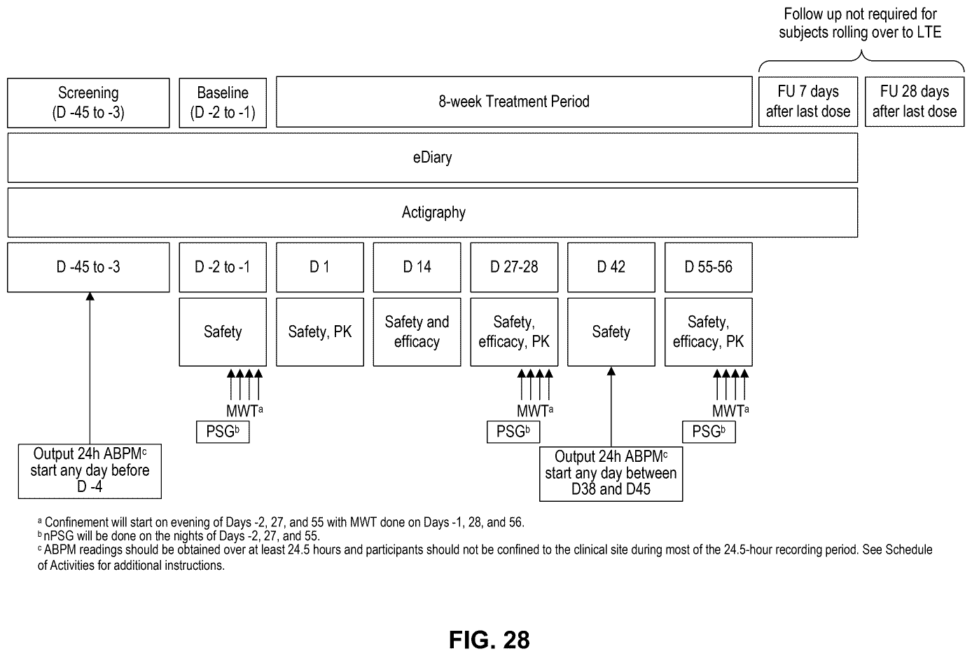

For a schematic of the study design, see FIG. 28.

Based on nonclinical data and clinical results for Compound A, clinical data of other compounds with the same mechanism of action, literature information on the association between OX2R agonism and cardiovascular effects, as well as effects on wakefulness in nonclinical models (Huang et al., 2010), potential risks for this product are:
Increases in BP and HR;
Insomnia; and
Bladder events (eg, micturition urgency, pollakiuria).

The principal mitigation strategy for risks related to BP increase, HR increase, insomnia, and bladder events included appropriate selection of the study population; use of the inpatient clinical research unit setting and home health visits, which permitted close monitoring and rapid institution of appropriate care as needed; appropriate specified monitoring procedures; and use of experienced staff trained in study procedures. To mitigate cardiovascular risks, BP and HR were measured frequently in this study; cardiovascular effects were evaluated by using BP and HR assessments and electrocardiographic (ECG) assessments. Stopping rules for individual participants and the overall study were established. In addition, because liver function test (LFT) elevation cases have been observed in another OX2R agonist with structural similarity, LFTs (ALT, AST, GGT, and total bilirubin) were closely monitored.

In addition, liver toxicity was observed with firazorexton (another OX2R agonist) in its phase 2 clinical studies. The structure of Compound A is closely related to firazorexton; however, as Compound A is considered to be a more potent OX2R agonist, the total daily doses used in this study to determine Compound A clinical safety, tolerability and efficacy profiles in NT2 were lower than the firazorexton doses. At the highest planned total daily dose of 7.0 mg, the Compound A covalent binding burden was projected to be lower than the previous OX2R agonist; hence the relative risk of clinical hepatotoxicity for Compound A was believed to be significantly lower for this OX2R agonist compound on the basis of its predicted low therapeutic dose.

No cases of drug-induced liver injury have been reported for Compound A. Additionally, appropriate mitigation measures were put in place in the protocols of the clinical studies to detect any liver enzyme elevations in a timely manner.

Liver function test (LFTs) (alanine aminotransferase [ALT], aspartate aminotransferase [AST], γ-glutamyl transferase [GGT], and total bilirubin) were closely monitored in this study. Stopping criteria for individual participants and the overall study, based on abnormal LFTs were established.

Finally, there was minimal risk associated with the noninvasive procedures planned for this study. Potential risks related to noninvasive study procedures included the following:

Acute hypersensitivity and/or anaphylactic reactions to new chemical entities are always a possible risk in any clinical study. Appropriate procedures were used to manage such possible risks.

Study procedure-specific risks included issues related to blood collection for safety and PK assessments (eg, venipuncture may cause bruising).

Review of available nonclinical and clinical data, including the nonserious, mild TEAEs reported in the Phase 1 trials supported a favorable benefit-risk ratio for this study with Compound A.

### Type of Participant and Disease Characteristics

The participant had an ICSD-3 diagnosis of NT2 by PSG/MSLT, performed within the past 5 years and meeting the minimal acceptable criteria for the proper performance of PSG/MSLT as outlined in the ICSD-3.

Note: If there was a potential participant with NT2 for whom a diagnostic nPSG/MSLT was performed more than 5 years ago or was not available, the site could repeat the diagnostic PSG/MSLT before Day −2.

### Exclusion Criteria

1. The participant had a current medical disorder, other than narcolepsy without cataplexy, associated with EDS.

2. The participant had a history of epilepsy, seizure, or convulsion, or had a family history of inherited disorders associated with seizure (except for a single febrile seizure in childhood).

3. The participant had one or more of the following psychiatric disorders:
a. Any current unstable psychiatric disorder.
b. Current or history of manic or hypomanic episode, schizophrenia or any other psychotic disorder, including schizoaffective disorder, major depression with psychotic features, bipolar depression with psychotic features, obsessive compulsive disorder, mental retardation, organic mental disorders, or mental disorders due to a general medical condition as defined in the Diagnostic and Statistical Manual of Mental Disorders, 5th Edition (DSM-5).
c. Current diagnosis or history of substance use disorder as defined in the DSM-5. Note: If the history of substance use disorder is more than 12 months before baseline, the participant could be allowed to enroll in the study after consultation with the sponsor or designee. (Participant must have also have negative urine drug screen at the screening and Day-2 visit).
d. Current active major depressive episode (MDE) or who have had an active MDE in the past 6 months. Note: Neurodevelopmental disorders (eg, attention deficit hyperactivity disease) were not excluded unless severity did not allow termination of prohibited medications.

4. The participant had a history of cerebral ischemia, transient ischemic attack (<5 years ago), intracranial aneurysm, or arteriovenous malformation.

5. The participant had major surgery or donated or lost 1 unit of blood (approximately 500 mL) within 4 weeks before the screening visit.

### Study Interventions Administered:

In this study, the interventions includes:
Dose Regimen 1:2 mg followed by 2 mg approximately 3 hours apart.
Dose Regimen 2:2 mg followed by 5 mg approximately 3 hours apart or 7 mg once daily.
Other products required for the study: placebo.

Dose regimens included once daily or twice daily (approximately 3 hours apart) dosing. Study treatment was administered at approximately 8 AM and 11 AM. (Participants assigned to a once daily dose regimen received placebo for the second dose.)

### Study Objectives and Endpoints

| | |
|---|---|
| Objectives | Endpoints |

Primary

| | |
|---|---|
| To assess the effect of Compound A | Change from baseline to |
| on excessive daytime sleepiness (EDS) | Week 8 in mean sleep |
| as measured by sleep latency from the | latency from the MWT. |
| Maintenance of Wakefulness Test (MWT). | |

Secondary

| | |
|---|---|
| To assess the effect of Compound A on | Change from baseline to |
| EDS as measured by the Epworth | Week 8 in ESS total score. |
| Sleepiness Scale (ESS) total score. | |
| To evaluate the safety and tolerability | Occurrence of at least 1 |
| of Compound A. | treatment-emergent adverse |
| | event (TEAE). |

### Representative Study Results

### Example 2A: Efficacy and Safety of Compound A

Eligible participants were age 18-70 years, with Epworth Sleepiness Scale (ESS) score>12 and no other current EDS-related medical conditions. Participants were randomized 1:1:1 to daily oral Compound A 2 mg twice 3 hours apart, 2 mg then 5 mg 3 hours later, or placebo. Study endpoints included change from baseline to Week 8 in mean Sleep Onset Latency on the Maintenance of Wakefulness Test (mean SOL-MWT; primary endpoint), ESS total score, and incidence of treatment-emergent adverse events (TEAEs). Participants who completed the placebo-controlled study could enroll in a ˜2-year Long Term Extension (LTE) study. LTE TEAE data up to Jan. 25, 2024 are reported.

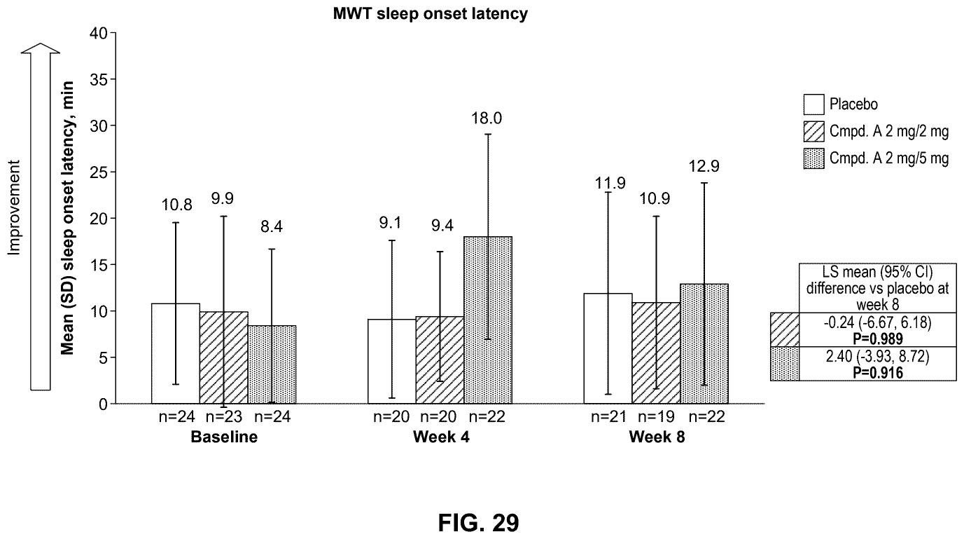

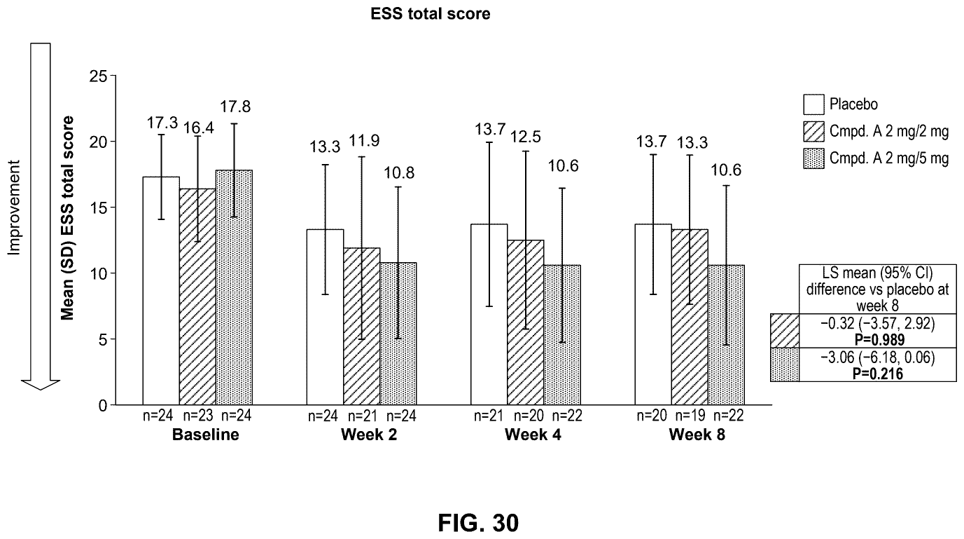

A total of 71 participants (mean age 36.2 years, MWT 9.7 minutes, and ESS score 17.1; 69.0% female) enrolled in the placebo-controlled study: 2 mg/2 mg (n=23); 2 mg/5 mg (n=24); placebo (n=24). FIG. 29 shows the change in SOL-MWT from baseline to week 8 for each group. Estimated mean changes from baseline to Week 8 in SOL-MWT were not statistically significant in the Compound A arms versus placebo (−0.24 and 2.4 minutes for 2 mg/2 mg and 2 mg/5 mg, both p>0.4). FIG. 30 shows the change in ESS from baseline to week 8 for each group. ESS total score was reduced from baseline to Week 8, but was not statistically significant versus placebo (−0.32 and −3.06 points for 2 mg/2 mg and 2/5 mg, both p>0.05). TEAEs occurred in 59.6% (28/47) of Compound A-treated versus 33.3% (8/24) of placebo-treated participants. Most A total of 58 participants enrolled in the LTE study (Example 3), of which, 57 were treated and 71.9% (41/57) experienced TEAEs. Most TEAEs were mild or moderate in severity and most were unrelated to study treatment. Two participants discontinued due to TEAEs in the parent study and 3 in the LTE study. There were no serious TEAEs during either study.

Conclusion: Compound A did not show statistically significant improvements versus placebo on measures of sleepiness over 8 weeks but was generally well tolerated in participants with NT2 for up to 6 months.

### Example 3. A Long-Term Extension Study to Evaluate the Safety and Tolerability of Compound A in Participants with Selected Central Hypersomnia Conditions

This was a phase 2/3 multicenter, dose-blind, long-term extension (LTE) study of Compound A. Participants from controlled studies conducted with Compound A, who had up to a 3-month dosing gap between their parent study and the screening period for this study, were eligible to participate in this study.

All required study assessments were briefly described below and were conducted as detailed in the schedule of activities. Participants rolling over directly into this study from a parent study without a dosing gap did not need to repeat those activities during screening if they were performed on the final visit of their parent study. Consent and eligibility were assessed during the final visit of the parent study; as such, these participants did not have an additional screening period and could proceed directly to Day 1 activities. For participants with a dosing gap, ESS, NSS-CT, and other patient-reported outcome scales were administered during screening.

Participants entering the current study with a dosing gap from the end date of their parent study had a screening period lasting up to 4 weeks; the exact duration depended on participant-specific medication washout requirements. During this period, participants underwent washout of narcolepsy medications and other restricted medications.

Day 1 dosing for all participants occurred in-clinic with PK and safety assessments. Participants who were undergoing nocturnal polysomnography (nPSG) and an MWT during the final assessment of their Compound A parent study had the option of staying overnight in the clinic on Day −1 so they were already in the clinic for Day 1 the following day. Clinic visits after Week 4 were scheduled monthly up to Week 12, then approximately every 3 months until the end of Year 2 (Weeks 2, 4, 8, 12, 26, 38, 52, 64, 78, 90, and 104). The higher in-clinic visit frequency during the first 12 weeks of the study ensured appropriate safety monitoring for participants who were previously on placebo and/or had a dosing gap. Participants had either an in-clinic or home health follow up visit 4 weeks after their last dose. All other visits could be conducted through home health. Laboratory tests for safety were obtained biweekly up until Week 12, followed by monthly until Year 1, and then approximately every 3 months thereafter. Monthly phone calls were conducted to collect adverse event (AE) and concomitant medication information in Year 2.

An nPSG and MWT were conducted on Day 181 and 182, respectively. Cognitive testing were conducted at Week 26 (6 months). ESS and NSS-CT were collected monthly starting at Week 4 and then approximately every 3 months after Week 12. There were additional ESS measurements at Week 2 and in the first week after drug discontinuation. PGI-I and CGI-I were collected approximately every 3 months starting at Week 12. Other patient-reported outcome measures were administered approximately every 6 months up until Year 1.

Throughout the study, participants complete electronic patient-reported diaries (e-diaries) to record daily dosing data, narcolepsy symptom and sleep data, and cataplexy data (participants with NT1 only). Participants with NT1 or NT2 entered daily Compound A dosing throughout the entire active treatment period, and narcolepsy symptom data during 2-week periods every 6 months up to Year 1. An actigraphy device was also worn by all participants during these same 2-week periods at approximately 6-month intervals up to Year 1.

Participants with NT1 also completed the Cataplexy e-diary daily over 2-week periods to capture the weekly cataplexy rate before the next visit in-clinic. The Cataplexy e-diary was completed at approximately 3-month intervals during Year 1 and at 6-month intervals during Year 2.

In this dose-blind study, all participants received Compound A. Participants entering the study with or without a dosing gap from a parent Compound A study remained on the same indication-specific dose of Compound A assigned to them in their parent study. Participants randomized to placebo or comparator in their parent study were randomized to one of the indication-specific dose regimens of Compound A available in this study. The investigator could request a change to either a higher or lower dose level of Compound A to be dispensed at Week 8 in the LTE treatment period. Subsequently, if the participant wished to return to their original LTE dose, this could be implemented at Week 12 in the LTE. No further dose adjustments were allowed for the duration of the study, except on a case by case basis as approved by the sponsor. The dose never exceeded the maximum designated dose level in the study. For a participant with NT1, if the next highest dose was the 7 mg total daily dose, the participant was randomized to 1 of those 2 dose regimens (i.e., 2 mg followed by 5 mg, or 7 mg once daily).

The dose could also be decreased to a lower dose level, but it could not be decreased below the lowest designated dose for that indication in the study. The dose change was automatically generated by the interactive response technology (IRT). If a request was made for dose adjustment and no higher or lower dose was available, the participant remained on their current blinded dose.

The doses at the start of the study included all prior doses from the phase 2 double-blind studies. If any doses were eliminated from the parent studies or in phase 3, the participants treated with the eliminated dose were switched to the next closest remaining dose available for that indication.

Standard safety endpoints (e.g., TEAEs, physical examination findings, vital signs, 12-lead ECG measures, clinical laboratory results) for early clinical investigation were included. Further, because cardiovascular effects had been noted in nonclinical models with Compound A and OX2R agonists in general, and these effects are thought to be on-target effects, close monitoring of cardiovascular parameters, including time-matched BP measurements before and after dosing to detect any changes and potential tolerance in BP after the administration of repeat doses to participants with NT1 or NT2 were included.

In addition, because cases of LFT elevation had been observed with another OX2R agonist with structural similarity, LFTs (including alanine aminotransferase [ALT), aspartate aminotransferase [AST], GGT, and total bilirubin) were closely monitored.

### Type of Participant and Disease Characteristics

Approximately 160 participants with a diagnosis of narcolepsy who had completed a controlled study with Compound A (including participants diagnosed with NT1 or NT2) and for whom the investigator had no clinical objection to their enrollment. Additionally, a rare exception could be granted by the sponsor or designee for a participant who was unable to complete a previous Compound A controlled study conducted in participants with narcolepsy.

### Exclusion Criteria

1. Participant had a moderate or severe ongoing TEAE related to the study drug from the parent study or discontinued because of TEAEs in the parent study.

2. Participant had ALT and AST>1.5 times the upper limit of normal (ULN) at multiple visits in the parent study and the findings were of clinical significance, per investigator or sponsor opinion, or ALT/AST>1.5 ULN during the screening period for participants with a dosing gap.

3. Participant had a positive urine screen for drugs of abuse (findings confirmed) and/or positive alcohol test during any visit in their prior Compound A study, or during the screening period for participants with a dosing gap. Products containing cannabidiol were allowed throughout the study, at the discretion of the investigator. Note: participants testing positive for marijuana at screening could be eligible for participation in the study provided that the principal investigator's clinical assessment indicated that the participant was not a regular user of marijuana, and after discussion with and approval from the sponsor or designee. Under this circumstance, a local urine dipstick drug screen was performed at the screening visit and verified to be negative before conducting any other study procedures at this visit. Any positive urine drug screens during conduct of the study had to be discussed with the sponsor or designee to determine the participant's disposition.

4. Participant was unable to discontinue, or refrain from using excluded medications for the duration of the study, including those used for the treatment of narcolepsy. Participant must have been willing to complete specified washout periods before the first dose of study drug

5. The participant had a current medical disorder, other than narcolepsy with or without cataplexy, associated with EDS.

6. Participant had current active major depressive episode (MDE) or had an active MDE in the past 6 months.

7. Participant had developed (within the last 6 months) gastrointestinal disease that was expected to influence the absorption of drugs (ie, a history of malabsorption, esophageal reflux, peptic ulcer disease, erosive esophagitis, frequent [more than once per week] occurrence of heartburn, or any surgical intervention).

8. Participant had epilepsy or history of seizure.

9. Participant had experienced clinically significant head injury, per investigator opinion.

10. Participant had any other medical condition, such as anxiety, depression, heart disease, or significant hepatic, pulmonary, or renal disease, that required them to take excluded medications.

11. Participant had a history of cerebral ischemia, transient ischemic attack (<5 years ago), or cerebral hemorrhage.

12. Participant had a history of myocardial infarction, clinically significant coronary artery disease, clinically significant angina, clinically significant cardiac rhythm abnormality, or heart failure.

13. Participant had a history of cancer in the past 5 years (does not apply to participants with carcinoma in situ that had been resolved without further treatment, or basal cell skin cancer; these participants could be included after approval by the sponsor or designee).

### Study Interventions Administered

The following dose regimens were included:

For participants with NT1:
Dose Regimen 1: 0.5 mg twice daily, approximately 3 hours apart;
Dose Regimen 2: 2 mg followed by 2 mg, approximately 3 hours apart;
Dose Regimen 3: 2 mg followed by 5 mg, approximately 3 hours apart; *
Dose Regimen 4: 7 mg once daily.*

For participants with NT2:
Dose Regimen 1: 2 mg followed by 2 mg, approximately 3 hours apart;
Dose Regimen 2: 2 mg followed by 5 mg, approximately 3 hours apart;** or
Dose Regimen 3: 7 mg once daily.
   * If a dose increase was requested from the 2 mg twice daily regimen, the participants were randomized to either of the 7 mg dosing options unless one of these doses was eliminated.
   ** Dose regimens included once daily or twice daily (approximately 3 hours apart) dosing. Study treatments were be administered at approximately 8:00 AM and 11:00 AM. (Participants assigned to a once daily dose regimen received placebo for the second dose.

### Study Objectives and Endpoints

| | |
|---|---|
| Objectives | Endpoints |

Primary

| | |
|---|---|
| To evaluate the long-term safety and | Occurrence of at least 1 treatment-emergent |
| tolerability of Compound A. | adverse event (TEAE). |

Secondary

| | |
|---|---|
| To assess the effect of Compound A on | Change from baseline in the parent study in |
| excessive daytime sleepiness (EDS) as | MWT mean sleep latency. |
| assessed by the mean sleep latency from the | |
| Maintenance of Wakefulness Test (MWT). | |
| To assess the effect of Compound A on EDS | Change from baseline in the parent study in |
| as measured by the Epworth Sleepiness | ESS total score. |
| Scale (ESS) total score. | |
| To assess the effect of Compound A on | Change from baseline in the parent study in |
| cataplexy as assessed by the weekly | WCR using the patient-reported cataplexy |
| cataplexy rate (WCR) (participants with | diary (participants with NT1 only). |
| narcolepsy type 1 [NT1] only). | |

Additional/Exploratory

| | |
|---|---|
| To assess the effect of Compound A on | PGI-I and CGI-I scores. |
| overall narcolepsy symptoms as measured by | |
| the Patient Global Impressions - | |
| Improvement (PGI-I) and Clinical Global | |
| Impressions -Improvement (CGI-I) scales. | |
| To assess the effect of Compound A on | Change from baseline in the parent study in |
| selected cognitive tests including the | PVT, CPAL, ONB, and iDSST-s. |
| Psychomotor Vigilance Test (PVT), | |
| Continuous Paired Associate Learning | |
| (CPAL) test, One Back test (ONB), and | |
| international Digit Symbol Substitution Test- | |
| symbols (iDSST-s). | |
| To assess the treatment satisfaction with | Treatment satisfaction as measured by the 4 |
| Compound A as measured by the Treatment | dimensions of the TSQM. |
| Satisfaction Questionnaire for Medication | |
| (TSQM). | |
| To assess the effect of Compound A on sleep | Change from baseline in the parent study in |
| architecture as measured by nocturnal | various nPSG measures including nocturnal |
| polysomnography (nPSG) data. | awakenings and rapid eye movement (REM) |
| | latency. |
| To assess the impact of Compound A on | Change from baseline in the parent study in |
| quality of life (QOL) of participants as | QOL as measured by the SF-36 score. |
| assessed by the | |
| 36-item Short Form-36 Survey (SF-36). | |
| To assess the effect of Compound A on | Change from baseline in the parent study in |
| mobility, self-care, usual activities, | EQ-5D-5L index score. |
| pain/discomfort, and anxiety/depression as | |
| assessed by the EuroQoL-5 Dimensions-5 | |
| Levels (EQ-5D-5L) scale. | |
| To assess the effect of Compound A on the | Change from baseline in the parent study in |
| severity of narcolepsy symptoms as | NSS-CT. |
| measured by the narcolepsy Severity Score | |
| for Clinical Trials (NSS-CT). | |
| To assess the effect of Compound A on | Change from baseline in the parent study in |
| functional impacts of narcolepsy as assessed | the FIN domain scores. |
| by the Functional Impacts of narcolepsy | |
| (FIN) scale. | |
| To evaluate additional safety measures of | Occurrence of at least 1 markedly abnormal |
| Compound A. | value (MAV) for postdose laboratory values. |
| | Occurrence of at least 1 MAV for postdose |
| | vital signs. |
| | Occurrence of at least 1 MAV for postdose |
| | electrocardiogram (ECG) parameters. |
| | Occurrence of at least 1 endorsement of Item |
| | 4 or 5 on the Columbia Suicide Severity |
| | Rating Scale (C-SSRS). |

### Representative Study Results

### Example 3A: Long-Term Safety and Efficacy of Compound A: Results from an Interim Analysis

Participants completing either Example 1 or Example 2 with ≤3 months between parent study-end and LTE screening could enroll. Primary endpoint is the occurrence of treatment-emergent adverse events (TEAEs); secondary endpoints are change from baseline (parent study) in sleep onset latency on the Maintenance of Wakefulness Test (SOL-MWT), Epworth Sleepiness Scale (ESS) score, and weekly cataplexy rate (WCR). Participants with NT1 receive oral Compound A 0.5 mg twice 3 hours apart, 2 mg twice 3 hours apart, 2 mg then 5 mg 3 hours apart, or 7 mg once daily. Dose adjustments are allowed at specific timepoints (8 weeks and 12 weeks of the study and at other weeks on a sponsor-approved case-by-case basis). TEAEs are summarized under the dose at TEAE onset.

Data up to Jan. 25, 2024 for NT1 participants (N=104) were included in this pre-specified interim analysis. All received Compound A 0.5 mg/0.5 mg (n=25), 2 mg/2 mg (n=25), 2 mg/5 mg (n=26), or 7 mg (n=28); 6 discontinued due to TEAEs (3 unrelated to Compound A). 54.8% of participants experienced a TEAE, the most frequent being insomnia (7.1%, 10.9%, 10.8%, and 7.9%), micturition frequency (10.7%, 4.3%, 8.1%, and 7.9%) and micturition urgency (10.7%, 2.2%, 2.7%, and 10.5%), across doses respectively. Two participants had severe drug-related TEAEs (insomnia, cataplexy). No cases of hepatotoxicity or visual disturbances were reported.

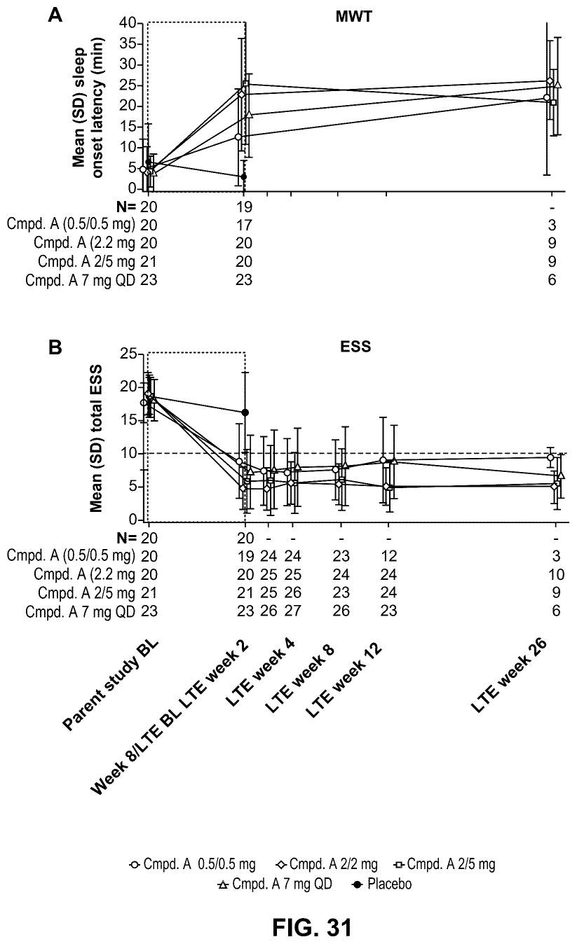

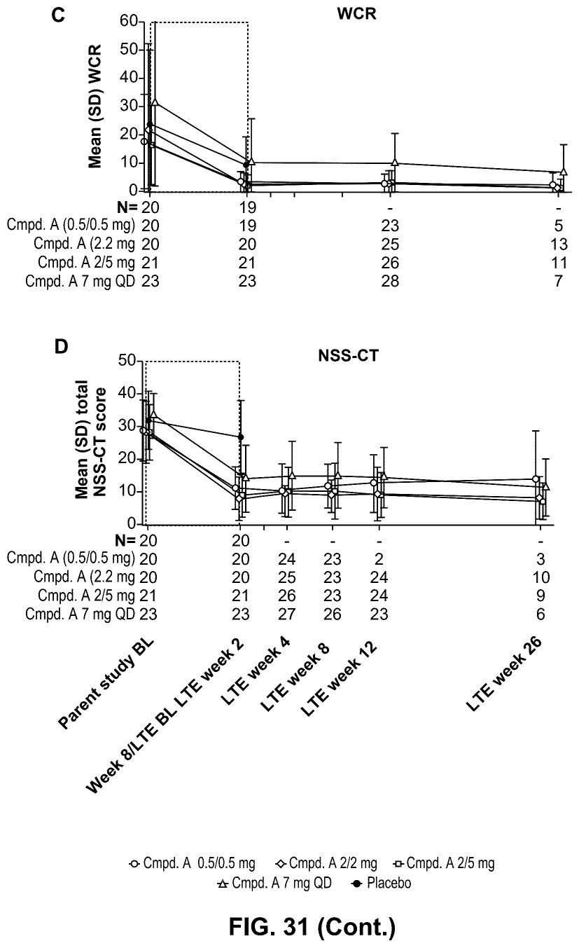

In this ongoing LTE study, interim data suggest maintenance of efficacy on objective (SOL-MWT) and subjective (ESS) assessments of EDS and WCR across all Compound A doses for up to 6 months. FIG. 31 shows results for all participants who received Compound A in the parent and LTE studies (excluding participants of the LTE who received placebo in the parent study), including those who had dose changes. Results were similar for the subset of participants who did not have a dose change in the LTE.

Conclusion: LTE interim results show Compound A was generally well tolerated and suggest efficacy was maintained up to 6 months in patients with NT1.

### Example 4: A Randomized, Double-Blind, Placebo-Controlled Study to Evaluate the Efficacy and Safety of Compound A for the Treatment of Narcolepsy with Cataplexy (Narcolepsy Type 1)

This study is a randomized, double-blind, placebo-controlled study investigating the efficacy and safety of Compound A in participants with NT1. Approximately 152 (male and female) participants with NT1 will be randomized 2:3:3 to placebo or 1 of 2 Compound A dose levels. Randomization will be stratified by region (North America, Europe, and Asia Pacific) and prior use of medication for narcolepsy. This study is designed to evaluate the efficacy and safety of Compound A in participants with NT1. This study compares Compound A with placebo. Each participant will have a 25% chance of being randomized to the placebo group and 75% chance of being randomized to Compound A. Placebo-controlled studies have commonly been used in drug approvals of other drugs to treat narcolepsy (Dauvilliers et al. 2013; US Xyrem Multicenter Study Group 2002). After completing this study, participants will have the option to participate in an LTE study, in which all participants receive active treatment.

Participants will be confined to an inpatient facility for specified periods throughout the conduct of the study. This confinement allows in-clinic assessments to be performed (e.g., overnight PSG), ensures adherence to in-clinic study procedures, and permits monitoring of safety.

### Study Population

### Age and Body Mass Index

1. The participant is aged 18 to 70 years, inclusive, at the time of signing the ICF. Note: Participants aged 16 and 17 years may be included in some countries/at some sites (e.g., Japan or US) based on local regulatory requirements.

2. The participant has a body mass index (BMI) within the range 18 to 40 kg/m2 (inclusive).

### Type of Participant and Disease Characteristics

3. The participant has an ICSD-3 or ICSD-3-TR diagnosis of NT1 supported by results from 1) PSG (with or without MSLT), performed within the past 15 years and meeting the minimal acceptable criteria for the proper performance of PSG/MSLT as outlined in the ICSD-3 or ICSD-3-TR, or 2) CSF test indicating an OX/hypocretin-1 concentration of ≤110 μg/mL (or less than one-third of the mean values obtained in normal participants within the same standardized assay).

Note: If there is a potential participant with NT1 for whom a diagnostic PSG/MSLT was performed more than 15 years from screening or if results are not available, the site may obtain a CSF sample or repeat the diagnostic PSG/MSLT before Day-2.

### ICSD-3 Diagnostic Criteria

Criteria a and b must be met:
a) The patient has daily periods of irrepressible need to sleep or daytime lapses into sleep occurring for at least 3 months. 1
b) The presence of 1 or both of the following:

1. Cataplexy (as defined under Essential Features) and a mean sleep latency of ≤8 minutes and 2 or more sleep onset REM periods (SOREMPs) on an MSLT performed according to standard techniques. A SOREMP (within 15 minutes of sleep onset) on the preceding nocturnal polysomnogram may replace one of the SOREMPs on the MSLT.2

2. CSF hypocretin-1 concentration, measured by immunoreactivity, is either ≤110 pg/ml or <⅓ of mean values obtained in normal participants with the same standardized assay. Notes: 1. In young children, narcolepsy may sometimes present as excessively long night sleep or as resumption of previously discontinued daytime napping. 2. If NT1 is strongly suspected clinically but the MSLT criteria of b1 are not met, a possible strategy is to repeat the MSLT.

### ICSD-3 TR Diagnostic Criteria

Criteria a, b, and c must be met.

a) The participant has daily periods of irrepressible need to sleep or daytime lapses into sleep.

b) The presence of one or both of the following:

1. Cataplexy (as defined under Essential Features) 1 and either: a. Mean sleep latency of ≤8 minutes and 2 or more SOREMPs on an MSLT performed in accordance with current recommended protocols2 or: b. A SOREMP (within 15 minutes of sleep onset) on nocturnal polysomnogram.

2. CSF hypocretin-1 concentration, measured by radioimmunoassay, is ≤110 μg/mL (using a Stanford reference sample) or less than one-third of mean values obtained in normal participants with the same standardized assay.3

c) The symptoms and signs are not better explained by chronic insufficient sleep, a circadian rhythm sleep-wake disorder or other current sleep disorder, medical disorder, mental disorder, or medication/substance use or withdrawal.

Notes: 1. Typical cataplexy is most strongly associated with NT1. Although some patients with NT1/hypocretin deficiency may present with atypical cataplexy features, presentations that include only atypical cataplexy should raise a higher index of doubt regarding a diagnosis of NT1. Clinical judgment is required. 2. Sleep logs are required, accompanied by actigraphy, whenever possible, before in laboratory sleep testing to evaluate for insufficient sleep and circadian rhythm disturbances. 3. If hypocretin deficiency is verified, the diagnosis of NT1 should be made regardless of other comorbidities that could potentially be related to clinical symptoms, given the definitive nature of this finding.

Further guidance is provided in the ICSD-3 or ICSD-3-TR guidelines.

4. The participant has an ESS score≥11 at screening.

5. The participant has >4 partial or complete episodes of cataplexy/week (WCR), calculated as the weekly average over 14 days (Days −16 to −3 of the screening period). Participants must complete self-reported cataplexy questions in the e-diary for at least 11 of 14 days during Days −16 to −3 to be considered compliant. In cases where the e-diary becomes unavailable, the study site may use alternative methods to collect these data with approval from the sponsor or designee.

6. The participant is positive for the HLA genotype HLA-DQB1*06:02 (positive results for either homozygous or heterozygous alleles will be considered “positive” and acceptable) or results from radioimmunoassay indicate the participant's CSF OX/hypocretin-1 concentration is ≤110 μg/mL (or less than one-third of the mean values obtained in normal participants within the same standardized assay).

Note: Previous HLA results are acceptable if available for review by the investigator and provided for inclusion in the electronic case report form.

### Exclusion Criteria

The participant will be excluded from the study if any of the following exclusion criteria are met:

### Medical Conditions

1. The participant has a current medical disorder, other than narcolepsy with cataplexy, associated with EDS.

2. The participant: (a) has a history of myocardial infarction; (b) has a history of clinically significant hepatic disease, thyroid disease, coronary artery disease, cardiac rhythm abnormality or heart failure; or (c) has any medical condition (such as unstable cardiovascular, pulmonary, renal or gastrointestinal disease, or an ongoing condition causing bladder outlet obstruction or urinary retention [eg, benign prostatic hyperplasia or pelvic organ prolapse with obstructive voiding symptoms, neurogenic bladder, or urethral stricture]) that would preclude enrollment in the view of the investigator.

3. The participant has current or recent (within 6 months) gastrointestinal disease that is expected to influence the absorption of drugs. Any history of Roux-en-Y gastric bypass is considered exclusionary, and any other surgical intervention that may influence the absorption of drugs should be discussed and approved by the sponsor or designee before enrolling the participant.

4. The participant has a history of cancer in the past 5 years (does not apply to participants with carcinoma in situ that has been resolved without further treatment or basal cell carcinoma; these participants may be included after approval by the sponsor or designee).

5. The participant has a clinically significant history of head injury or head trauma.

6. The participant has a history of epilepsy, seizure, or convulsion (except for a single febrile seizure in childhood).

7. The participant has a history of cerebral ischemia, transient ischemic attack (<5 years from screening), intracranial aneurysm, or arteriovenous malformation.

8. The participant has 1 or more of the following psychiatric disorders:
a) Current or history of manic or hypomanic episode; schizophrenia, or any other psychotic disorder, including schizoaffective disorder, major depression with psychotic features, and bipolar depression with psychotic features; obsessive compulsive disorder; intellectual disability; organic mental disorders; or mental disorders due to a general medical condition as defined in the Diagnostic and Statistical Manual of Mental Disorders, 5th Edition (DSM-5).
b) Current diagnosis or history of substance use disorder as defined in the DSM-5.
c) Current active major depressive episode (MDE) or an active MDE in the past 6 months.
d) Any current unstable psychiatric disorder.

Note: Neurodevelopmental disorders (e.g., attention deficit hyperactivity disorder) are not excluded unless severity does not allow termination of prohibited medications

9. The participant has had major surgery or donated or lost 1 unit of blood (approximately 500 mL) within 4 weeks before the screening visit.

10. The participant has ALT or AST values>1.5 times the ULN or GGT>3 times the ULN at screening or Day −2 (if results available). The participant must have at least one AST, ALT, and GGT result available from ≤16 days before randomization.

11. The participant has a positive urine screen result for drugs of abuse and/or positive alcohol test result at screening or Day-2. An exception at screening is made for stimulants or other drugs the participant has been prescribed. Products containing cannabidiol (but not tetrahydrocannabinol) may be allowed throughout the study, at the discretion of the investigator.

12. The participant is considered by the investigator to be at imminent risk of suicide or injury to self, others, or property, or the participant has attempted suicide within the past year before screening or has positive answers on Item 4 or 5 on the CSSRS within a year before randomization.

### Study Intervention(s) Administered

In this study, the interventions include:
Compound A Dose Level 1 (1 mg BID at least 3 hours apart).
Compound A Dose Level 2 (2 mg BID at least 3 hours apart).
Matching placebo (BID at least 3 hours apart).

To maintain the blind, all participants, irrespective of dose level, will receive the same number of tablets and bottles.

### Administration

Starting on the morning of Day 1, the study drug will be administered at approximately the same time each day for 12 weeks.

On Clinic Days 1, 56, and 84, the first dose of Compound A or matched placebo will be administered in the morning by mouth (as close to 8 AM as possible) with 240 mL of water after an overnight fast of at least 8 hours; participants will then be allowed to have a morning meal after study drug administration. Participants may consume water ad libitum. Participants will take the second dose at least 3 hours after the first morning dose (as close to 11 AM as possible).

On ABPM fitting days participants should take their study drug right after the ABPM device begins recording (no later than 10 AM). The ABPM device will be applied on-site. Because it is a requirement that the ABPM devices begin recording in the morning before the first morning dose, site staff may opt to confine participants the evening before fitting participants with the ABPM monitoring device. If the participant is travelling to the site for the ABPM fitting, they may delay their first morning dose of study drug (but taken no later than 10 AM) if needed. Participants who are confined the evening prior should also take their study drug right after the ABPM recording starts.

On other clinic days and while at home, participants will be instructed to take the first dose of Compound A/matched placebo in the morning, no later than 10 AM, with a large glass of water (approximately 240 mL total). Participants will be instructed to take the second dose at least 3 hours after the first morning dose, but no later than 1 PM. Participants are encouraged to take study drug at approximately the same time each day.

Participants should swallow the study drug whole and not chew it or manipulate it in any way before swallowing. Participants should be instructed not to take more than the prescribed dose at any time. If a dose window is missed, the dose should be skipped, and no drug should be taken until the next scheduled dose. Under no circumstance should a participant repeat a dose or double-up doses.

Additional steps may be taken to ensure participants understand the dosing instructions and that they follow the correct Compound A dosing level, such as additional site communication with the participant throughout the treatment course, i.e., on-site visits or phone calls.

Within the source documents, site personnel should document instruction of and understanding by the participant of the safe, responsible storage and administration of study intervention to the study participant.

After the completion of the Day 84 Visit in the current study, participants who remained on study treatment throughout the treatment period will have the option to participate in an LTE study under a separate protocol (assuming the protocol is open for enrollment).

### Study Objectives and Endpoints

| | |
|---|---|
| Objectives | Endpoints |

Primary

| | |
|---|---|
| To assess the effect of Compound A on EDS | Mean sleep latency from the MWT at Week |
| as measured by the MWT. | 12. |

Secondary

| | |
|---|---|
| To assess the effect of Compound A on EDS | ESS total score at Week 12. |
| as measured by the ESS total score. | |
| To assess the effect of Compound A on | WCR at Week 12. |
| cataplexy as measured by the WCR. | |
| To assess the effect of Compound A on | Number of lapses on the 3 PM PVT session |
| sustained attention as measured by the | at Week 12. |
| psychomotor vigilance test (PVT). | |
| To assess the effect of Compound A on | PGI-C score at Week 12. |
| overall narcolepsy symptoms measured by | |
| the Patient Global Impression of Change | |
| (PGI-C) scale. | |
| To assess the effect of Compound A on | NSS-CT total score at Week 12. |
| overall severity of narcolepsy symptoms | |
| measured by the narcolepsy Severity Scale | |
| for Clinical Trials (NSS-CT). | |
| To assess the efficacy of Compound A on | FINI domain scores at Week 12 for: |
| functional impacts of narcolepsy as assessed | Tiredness, Cognitive Functioning, |
| by the Functional Impacts of narcolepsy | Cataplexy, Social Activities, Everyday |
| Instrument (FINI). | Activities, and Everyday Responsibilities. |
| To assess the effect of Compound A on | SF-36 mental and physical component |
| quality of life of participants, as assessed by | summary scores at Week 12. |
| the Short Form-36 Survey (SF-36). | |
| To evaluate the safety of Compound A as | Occurrence of at least 1 TEAE during the |
| measured by TEAE incidence. | study including the follow-up period, as |
| | applicable. |

Additional/Exploratory

| | |
|---|---|
| To evaluate additional safety measures of | Occurrence of at least 1 MAV for postdose |
| Compound A. | laboratory values. |
| | Occurrence of at least 1 MAV for postdose |
| | vital signs. |
| | Occurrence of at least 1 MAV for postdose |
| | ECG parameters. |
| | Time-matched change in BP and HR from |
| | baseline to specified postdose time points. |
| | Change in ambulatory BP and HR |
| | parameters from baseline (ambulatory BP |
| | monitoring [ABPM]). |
| | Occurrence of at least 1 endorsement of Item |
| | 4 or 5 on the Columbia Suicide Severity |
| | Rating Scale (CSSRS). |
| To assess the effect of Compound A on | CGI-C score. |
| overall narcolepsy symptoms measured by | |
| the Clinical Global Impression of Change | |
| (CGI-C) scale. | |
| To assess the effect of Compound A on | CGI-S and PGI-S score. |
| Clinical Global Impression - Severity of | |
| Illness (CGI-S) scale and Patient Global | |
| Impression - Severity (PGI-S) scale. | |
| To assess the effect on Compound A on | NSS-CT individual question scores. |
| severity of narcolepsy symptoms measured | |
| by the NSS-CT. | |
| To assess the effect of Compound A on | nPSG measures, including nocturnal |
| sleep architecture as measured by nocturnal | awakenings and sleep efficiency. |
| polysomnography (nPSG). | |
| To assess the effect of Compound A on | Total sleep time and responses to nocturnal |
| quantity and quality of nocturnal sleep. | sleep quantity and quality questions as |
| | reported in the daily electronic patient- |
| | reported diary (e-diary). |
| To assess the effects of Compound A on | CPAL, ONB, and iDSST-s outcome |
| memory, working memory, and processing | measures. |
| speed as measured by the cognitive tests | |
| (Continuous Paired Associate Learning | |
| [CPAL] test, One Back test [ONB], and | |
| international Digit Symbol Substitution | |
| Test-symbols [iDSST-s]). | |
| To assess the effect of Compound A on | KSS scores. |
| sleepiness, as measured by as measured by | |
| the KSS. | |
| To assess the effect of Compound A on | BC CCI-E scores. |
| perceived cognitive difficulties and the | |
| functional and quality of life impacts of | |
| cognitive complaints as assessed by the | |
| British Columbia Cognitive Complaints | |
| Inventory expanded version (BC CCI-E). | |
| To assess the treatment satisfaction with | Treatment satisfaction as measured by the 4 |
| Compound A as measured by the Treatment | dimensions of the TSQM: effectiveness, side |
| Satisfaction Questionnaire for Medication | effects, convenience, and global satisfaction. |
| (TSQM) which captures 4 domains | |
| addressing effectiveness, side effects, | |
| convenience, and global satisfaction. | |
| To assess the effect of Compound A on | EQ-5D-5L index and VAS scores. |
| mobility, self-care, usual activities, | |
| pain/discomfort, and anxiety/depression as | |
| assessed by the EuroQol-5 Dimensions 5- | |
| Levels (EQ-5D-5L) scale. | |
| To assess the effect of Compound A on | Eight SF-36 domain scores. |
| quality of life of participants, as assessed by | |
| the SF-36. | |

### Estimands

The following are the objectives and associated estimands for this study:

| Objective | Estimand |
|---|---|
| Primary objective: | Primary Estimand: The treatment effect of |
| To assess the effect of Compound A on EDS as | Compound A compared to placebo on EDS as |
| measured by the MWT. | measured by mean sleep latency from the MWT at |
| | Week 12 in the targeted patient population. |
| | Endpoint: Mean sleep latency from the MWT at |
| | Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the effect of Compound A on EDS as | Compound A compared to placebo on EDS as |
| measured by the ESS total score. | measured by ESS total score at Week 12 in the |
| | targeted patient population. |
| | Endpoint: ESS total score at Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the effect of Compound A on cataplexy | Compound A compared to placebo on cataplexy |
| as measured by the WCR. | as measured by the WCR at Week 12 in the |
| | targeted patient population. |
| | Endpoint: WCR at Week 12 |
| | Population level summary: The incidence rate of |
| | the variable will be estimated for each treatment |
| | group and comparison to placebo will be based on |
| | the ratio of incidence rates between treatment |
| | groups. |
| Secondary objective: | Secondary Estimand: The treatment effect of |
| To assess the effect of Compound A on sustained | Compound A compared to placebo on sustained |
| attention as measured by the PVT. | attention as measured by the PVT at Week 12 in |
| | the targeted patient population. |
| | Endpoint: Number of lapses on the 3PM PVT |
| | session at Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the effect of Compound A on overall | Compound A compared to placebo on overall |
| narcolepsy symptoms measured by the PGI-C | narcolepsy symptoms as measured by the PGI-C |
| scale. | at Week 12 in the targeted patient |
| | population. |
| | Endpoint: PGI-C score at Week 12 |
| | Population level summary: The proportion of |
| | participants reporting much or very much |
| | improved overall narcolepsy symptoms will be |
| | estimated for each treatment group and |
| | comparison to placebo will be based on the odds |
| | ratio of reporting much or very much improved |
| | overall narcolepsy symptoms between treatment |
| | groups. |
| Secondary objective: | Secondary Estimand: The treatment effect of |
| To assess the effect of Compound A on overall | Compound A compared to placebo on overall |
| severity of narcolepsy symptoms measured by the | severity of narcolepsy symptoms as measured by |
| NSS-CT. | the NSS-CT at Week 12 in the targeted patient |
| | population. |
| | Endpoint: NSS-CT total score at Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the efficacy of Compound A on | Compound A compared to placebo on functional |
| functional impacts of narcolepsy as assessed by | impacts of narcolepsy as assessed by the FINI at |
| the FINI. | Week 12 in the targeted patient population. |
| | Endpoints: FINI domain scores at Week 12 for: |
| | Tiredness, Cognitive Functioning, Cataplexy, |
| | Social Activities, Everyday Activities, and |
| | Everyday Responsibilities |
| | Population level summary: The mean of the |
| | score for each domain will be estimated for each |
| | treatment group and comparison to placebo for |
| | each domain will be based on the difference in |
| | means between treatment groups. |
| Secondary objective: | Secondary Estimand: The treatment effect of |
| To assess the effect of Compound A on quality of | Compound A compared to placebo on quality of |
| life of participants, as assessed by the SF-36. | life of participants as assessed by the SF-36 at |
| | Week 12 in the targeted patient population. |
| | Endpoints: SF-36 component summary scores at |
| | Week 12 for physical and mental components |
| | Population level summary: The mean of the |
| | score for each component will be estimated for |
| | each treatment group and comparison to placebo |
| | for each component will be based on the |
| | difference in means between treatment groups. |

### Example 5: A Randomized, Double-Blind, Placebo-Controlled Study to Evaluate the Efficacy and Safety of Compound A for the Treatment of Narcolepsy with Cataplexy (Narcolepsy Type 1)

This study is a randomized, double-blind, placebo-controlled study investigating the efficacy and safety of Compound A in participants with NT1. Approximately 93 (male and female) participants with NT1 will be randomized 1:2 to placebo or Compound A, 2 mg twice daily. Randomization will be stratified by region (such as Europe, and Asia Pacific) and prior use of medication for narcolepsy. This study is designed to evaluate the efficacy and safety of Compound A in participants with NT1. This study compares Compound A with placebo. Each participant will have a 1 out of 3 chance of being randomized to the placebo group and a 2 out of 3 chance of being randomized to Compound A. After completing this study, participants will have the option to participate in an LTE study, in which all participants receive active treatment.

Participants will be confined to an inpatient facility for specified periods throughout the conduct of the study. This confinement allows in-clinic assessments to be performed (e.g., overnight PSG), ensures adherence to in-clinic study procedures, and permits monitoring of safety.

### Study Population

### Age and Body Mass Index

1. The participant is aged 18 to 70 years, inclusive, at the time of signing the ICF. Note: Participants aged 16 and 17 years may be included in some countries/at some sites (e.g., Japan or US) based on local regulatory requirements.

2. The participant has a body mass index (BMI) within the range 18 to 40 kg/m2 (inclusive).

### Type of Participant and Disease Characteristics

3. The participant has an ICSD-3 or ICSD-3-TR diagnosis of NT1 supported by results from 1) PSG (with or without MSLT), performed within the past 15 years and meeting the minimal acceptable criteria for the proper performance of PSG/MSLT as outlined in the ICSD-3 or ICSD-3-TR, or 2) CSF test indicating an OX/hypocretin-1 concentration of ≤110 pg/mL (or less than one-third of the mean values obtained in normal participants within the same standardized assay).

Note: If there is a potential participant with NT1 for whom a diagnostic PSG/MSLT was performed more than 15 years from screening or if results are not available, the site may obtain a CSF sample or repeat the diagnostic PSG/MSLT before Day-2.

### ICSD-3 Diagnostic Criteria

Criteria a and b must be met:

a) The patient has daily periods of irrepressible need to sleep or daytime lapses into sleep occurring for at least 3 months. 1

b) The presence of 1 or both of the following:

1. Cataplexy (as defined under Essential Features) and a mean sleep latency of ≤8 minutes and 2 or more sleep onset REM periods (SOREMPs) on an MSLT performed according to standard techniques. A SOREMP (within 15 minutes of sleep onset) on the preceding nocturnal polysomnogram may replace one of the SOREMPs on the MSLT.2

2. CSF hypocretin-1 concentration, measured by immunoreactivity, is either ≤110 μg/ml or <⅓ of mean values obtained in normal participants with the same standardized assay. Notes: 1. In young children, narcolepsy may sometimes present as excessively long night sleep or as resumption of previously discontinued daytime napping. 2. If NT1 is strongly suspected clinically but the MSLT criteria of b1 are not met, a possible strategy is to repeat the MSLT.

### ICSD-3 TR Diagnostic Criteria

Criteria a, b, and c must be met.

a) The participant has daily periods of irrepressible need to sleep or daytime lapses into sleep.

b) The presence of one or both of the following:

1. Cataplexy (as defined under Essential Features) 1 and either: a. Mean sleep latency of ≤8 minutes and 2 or more SOREMPs on an MSLT performed in accordance with current recommended protocols2 or: b. A SOREMP (within 15 minutes of sleep onset) on nocturnal polysomnogram.

2. CSF hypocretin-1 concentration, measured by radioimmunoassay, is ≤110 pg/mL (using a Stanford reference sample) or less than one-third of mean values obtained in normal participants with the same standardized assay 3.

c) The symptoms and signs are not better explained by chronic insufficient sleep, a circadian rhythm sleep-wake disorder or other current sleep disorder, medical disorder, mental disorder, or medication/substance use or withdrawal.

Notes: 1. Typical cataplexy is most strongly associated with NT1. Although some patients with NT1/hypocretin deficiency may present with atypical cataplexy features, presentations that include only atypical cataplexy should raise a higher index of doubt regarding a diagnosis of NT1. Clinical judgment is required. 2. Sleep logs are required, accompanied by actigraphy, whenever possible, before in-laboratory sleep testing to evaluate for insufficient sleep and circadian rhythm disturbances. 3. If hypocretin deficiency is verified, the diagnosis of NT1 should be made regardless of other comorbidities that could potentially be related to clinical symptoms, given the definitive nature of this finding.

Further guidance is provided in the ICSD-3 or ICSD-3-TR guidelines.

4. The participant has an ESS score≥11 at screening.

5. The participant has ≥4 partial or complete episodes of cataplexy/week (WCR), calculated as the weekly average over 14 days (Days −16 to −3 of the screening period). Participants must complete self-reported cataplexy questions in the e-diary for at least 11 of 14 days during Days −16 to −3 to be considered compliant. In cases where the e-diary becomes unavailable, the study site may use alternative methods to collect these data with approval from the sponsor or designee.

6. The participant is positive for the HLA genotype HLA-DQB1*06:02 (positive results for either homozygous or heterozygous alleles will be considered “positive” and acceptable) or results from radioimmunoassay indicate the participant's CSF OX/hypocretin-1 concentration is ≤110 pg/mL (or less than one-third of the mean values obtained in normal participants within the same standardized assay).

Note: Previous HLA results are acceptable if available for review by the investigator and provided for inclusion in the electronic case report form.

### Exclusion Criteria

The participant will be excluded from the study if any of the following exclusion criteria are met:

### Medical Conditions

1. The participant has a current medical disorder, other than narcolepsy with cataplexy, associated with EDS.

2. The participant: (a) has a history of myocardial infarction; (b) has a history of clinically significant hepatic disease, thyroid disease, coronary artery disease, cardiac rhythm abnormality or heart failure; or (c) has any medical condition (such as unstable cardiovascular, pulmonary, renal or gastrointestinal disease, or an ongoing condition causing bladder outlet obstruction or urinary retention [e.g., benign prostatic hyperplasia or pelvic organ prolapse with obstructive voiding symptoms, neurogenic bladder, or urethral stricture]) that would preclude enrollment in the view of the investigator.

3. The participant has current or recent (within 6 months) gastrointestinal disease that is expected to influence the absorption of drugs. Any history of Roux-en-Y gastric bypass is considered exclusionary, and any other surgical intervention that may influence the absorption of drugs should be discussed and approved by the sponsor or designee before enrolling the participant.

4. The participant has a history of cancer in the past 5 years (does not apply to participants with carcinoma in situ that has been resolved without further treatment or basal cell carcinoma; these participants may be included after approval by the sponsor or designee).

5. The participant has a clinically significant history of head injury or head trauma.

6. The participant has a history of epilepsy, seizure, or convulsion (except for a single febrile seizure in childhood).

7. The participant has a history of cerebral ischemia, transient ischemic attack (<5 years from screening), intracranial aneurysm, or arteriovenous malformation.

8. The participant has 1 or more of the following psychiatric disorders:
a) Current or history of manic or hypomanic episode; schizophrenia, or any other psychotic disorder, including schizoaffective disorder, major depression with psychotic features, and bipolar depression with psychotic features; obsessive compulsive disorder; intellectual disability; organic mental disorders; or mental disorders due to a general medical condition as defined in the Diagnostic and Statistical Manual of Mental Disorders, 5th Edition (DSM-5).
b) Current diagnosis or history of substance use disorder as defined in the DSM-5.
c) Current active major depressive episode (MDE) or an active MDE in the past 6 months.
d) Any current unstable psychiatric disorder.

Note: Neurodevelopmental disorders (e.g., attention deficit hyperactivity disorder) are not excluded unless severity does not allow termination of prohibited medications

9. The participant has had major surgery or donated or lost 1 unit of blood (approximately 500 mL) within 4 weeks before the screening visit.

### Prior/Concomitant Therapy

10. The participant has ALT or AST values>1.5 times the ULN or GGT>3 times the ULN at screening or Day-2 (if results available). The participant must have at least one AST, ALT, and GGT result available from ≤16 days before randomization.

11. The participant has a positive urine screen result for drugs of abuse and/or positive alcohol test result at screening or Day-2. An exception at screening is made for stimulants or other drugs the participant has been prescribed. Products containing cannabidiol (but not tetrahydrocannabinol) may be allowed throughout the study, at the discretion of the investigator.

12. The participant is considered by the investigator to be at imminent risk of suicide or injury to self, others, or property, or the participant has attempted suicide within the past year before screening or has positive answers on Item 4 or 5 on the CSSRS within a year before randomization.

### Study Intervention(s) Administered

In this study, the interventions include:
Compound A, 2 mg BID at least 3 hours apart.
Matching placebo (BID at least 3 hours apart).

To maintain the blind, all participants, irrespective of dose level, will receive the same number of tablets and bottles.

### Administration

Starting on the morning of Day 1, the study drug will be administered at approximately the same time each day for 12 weeks.

On Clinic Days 1, 56, and 84, the first dose of Compound A or matched placebo will be administered in the morning by mouth (as close to 8 AM as possible) with 240 mL of water after an overnight fast of at least 8 hours; participants will then be allowed to have a morning meal after study drug administration. Participants may consume water ad libitum. Participants will take the second dose at least 3 hours after the first morning dose (as close to 11 AM as possible).

On ABPM fitting days participants should take their study drug right after the ABPM device begins recording (no later than 10 AM). The ABPM device will be applied on-site. Because it is a requirement that the ABPM devices begin recording in the morning before the first morning dose, site staff may opt to confine participants the evening before fitting participants with the ABPM monitoring device. If the participant is travelling to the site for the ABPM fitting, they may delay their first morning dose of study drug (but taken no later than 10 AM) if needed. Participants who are confined the evening prior should also take their study drug right after the ABPM recording starts.

On other clinic days and while at home, participants will be instructed to take the first dose of Compound A/matched placebo in the morning, no later than 10 AM, with a large glass of water (approximately 240 mL total). Participants will be instructed to take the second dose at least 3 hours after the first morning dose, but no later than 1 PM. Participants are encouraged to take study drug at approximately the same time each day.

Participants should swallow the study drug whole and not chew it or manipulate it in any way before swallowing. Participants should be instructed not to take more than the prescribed dose at any time. If a dose window is missed, the dose should be skipped, and no drug should be taken until the next scheduled dose. Under no circumstance should a participant repeat a dose or double-up doses.

Additional steps may be taken to ensure participants understand the dosing instructions and that they follow the correct Compound A dosing level, such as additional site communication with the participant throughout the treatment course, i.e., on-site visits or phone calls.

Within the source documents, site personnel should document instruction of and understanding by the participant of the safe, responsible storage and administration of study intervention to the study participant.

After the completion of the Day 84 Visit in the current study, participants who remained on study treatment throughout the treatment period will have the option to participate in an LTE study under a separate protocol (assuming the protocol is open for enrollment).

### Study Objectives and Endpoints

| | |
|---|---|
| Objectives | Endpoints |

Primary

| | |
|---|---|
| To assess the effect of Compound A on EDS | Mean sleep latency from the MWT at Week |
| as measured by the MWT. | 12. |

Secondary

| | |
|---|---|
| To assess the effect of Compound A on EDS | ESS total score at Week 12. |
| as measured by the ESS total score. | |
| To assess the effect of Compound A on | WCR at Week 12. |
| cataplexy as measured by the WCR. | |
| To assess the effect of Compound A on | Number of lapses on the 3 PM PVT session |
| sustained attention as measured by the | at Week 12. |
| psychomotor vigilance test (PVT). | |
| To assess the effect of Compound A on | PGI-C score at Week 12. |
| overall narcolepsy symptoms measured by | |
| the Patient Global Impression of Change | |
| (PGI-C) scale. | |
| To assess the effect of Compound A on | NSS-CT total score at Week 12. |
| overall severity of narcolepsy symptoms | |
| measured by the narcolepsy Severity Scale | |
| for Clinical Trials (NSS-CT). | |
| To assess the efficacy of Compound A on | FINI domain scores at Week 12 for: |
| functional impacts of narcolepsy as assessed | Tiredness, Cognitive Functioning, |
| by the Functional Impacts of narcolepsy | Cataplexy, Social Activities, Everyday |
| Instrument (FINI). | Activities, and Everyday Responsibilities. |
| To assess the effect of Compound A on | SF-36 mental and physical component |
| quality of life of participants, as assessed by | summary scores at Week 12. |
| the Short Form-36 Survey (SF-36). | |
| To evaluate the safety of Compound A as | Occurrence of at least 1 TEAE during the |
| measured by TEAE incidence. | study including the follow-up period, as |
| | applicable. |

Additional/Exploratory

| | |
|---|---|
| To evaluate additional safety measures of | Occurrence of at least 1 MAV for postdose |
| Compound A. | laboratory values. |
| | Occurrence of at least 1 MAV for postdose |
| | vital signs. |
| | Occurrence of at least 1 MAV for postdose |
| | ECG parameters. |
| | Time-matched change in BP and HR from |
| | baseline to specified postdose time points. |
| | Change in ambulatory BP and HR |
| | parameters from baseline (ambulatory BP |
| | monitoring [ABPM]). |
| | Occurrence of at least 1 endorsement of Item |
| | 4 or 5 on the Columbia Suicide Severity |
| | Rating Scale (CSSRS). |
| To assess the effect of Compound A on | CGI-C score. |
| overall narcolepsy symptoms measured by | |
| the Clinical Global Impression of Change | |
| (CGI-C) scale. | |
| To assess the effect of Compound A on | CGI-S and PGI-S score. |
| Clinical Global Impression - Severity of | |
| Illness (CGI-S) scale and Patient Global | |
| Impression - Severity (PGI-S) scale. | |
| To assess the effect on Compound A on | NSS-CT individual question scores. |
| severity of narcolepsy symptoms measured | |
| by the NSS-CT. | |
| To assess the effect of Compound A on | nPSG measures, including nocturnal |
| sleep architecture as measured by nocturnal | awakenings and sleep efficiency. |
| polysomnography (nPSG). | |
| To assess the effect of Compound A on | Total sleep time and responses to nocturnal |
| quantity and quality of nocturnal sleep. | sleep quantity and quality questions as |
| | reported in the daily electronic patient- |
| | reported diary (e-diary). |
| To assess the effects of Compound A on | CPAL, ONB, and iDSST-s outcome |
| memory, working memory, and processing | measures. |
| speed as measured by the cognitive tests | |
| (Continuous Paired Associate Learning | |
| [CPAL] test, One Back test [ONB], and | |
| international Digit Symbol Substitution | |
| Test-symbols [iDSST-s]). | |
| To assess the effect of Compound A on | KSS scores. |
| sleepiness, as measured by as measured by | |
| the KSS | |
| To assess the effect of Compound A on | BC CCI-E scores. |
| perceived cognitive difficulties and the | |
| functional and quality of life impacts of | |
| cognitive complaints as assessed by the | |
| British Columbia Cognitive Complaints | |
| Inventory expanded version (BC CCI-E). | |
| To assess the treatment satisfaction with | Treatment satisfaction as measured by the 4 |
| Compound A as measured by the Treatment | dimensions of the TSQM: effectiveness, side |
| Satisfaction Questionnaire for Medication | effects, convenience, and global satisfaction. |
| (TSQM) which captures 4 domains | |
| addressing effectiveness, side effects, | |
| convenience, and global satisfaction. | |
| To assess the effect of Compound A on | EQ-5D-5L index and VAS scores. |
| mobility, self-care, usual activities, | |
| pain/discomfort, and anxiety/depression as | |
| assessed by the EuroQol-5 Dimensions 5- | |
| Levels (EQ-5D-5L) scale. | |
| To assess the effect of Compound A on | Eight SF-36 domain scores. |
| quality of life of participants, as assessed by | |
| the SF-36. | |
| To assess the effect of Compound A on | WPAI:SHP scores. |
| work performance by the Work | |
| Productivity and Activity Impairment | |
| Questionnaire:Specific Health Problem | |
| (WPAI:SHP) | |
| To assess steady-state exposures of | Compound A plasma concentrations. |
| Compound A. | |
| To assess the PK/PD relationship(s) of | |
| Compound A for selected efficacy or safety | |
| measures. | |

### Estimands

The following are the objectives and associated estimands for this study:

| Objective | Estimand |
|---|---|
| Primary objective: | Primary Estimand: The treatment effect of |
| To assess the effect of Compound A on EDS as | Compound A compared to placebo on EDS as |
| measured by the MWT. | measured by mean sleep latency from the MWT at |
| | Week 12 in the targeted patient population. |
| | Endpoint: Mean sleep latency from the MWT at |
| | Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the effect of Compound A on EDS as | Compound A compared to placebo on EDS as |
| measured by the ESS total score. | measured by ESS total score at Week 12 in the |
| | targeted patient population. |
| | Endpoint: ESS total score at Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the effect of Compound A on cataplexy | Compound A compared to placebo on cataplexy |
| as measured by the WCR. | as measured by the WCR at Week 12 in the |
| | targeted patient population. |
| | Endpoint: WCR at Week 12 |
| | Population level summary: The incidence rate of |
| | the variable will be estimated for each treatment |
| | group and comparison to placebo will be based on |
| | the ratio of incidence rates between treatment |
| | groups. |
| Secondary objective: | Secondary Estimand: The treatment effect of |
| To assess the effect of Compound A on sustained | Compound A compared to placebo on sustained |
| attention as measured by the PVT. | attention as measured by the PVT at Week 12 in |
| | the targeted patient population. |
| | Endpoint: Number of lapses on the 3PM PVT |
| | session at Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the effect of Compound A on overall | Compound A compared to placebo on overall |
| narcolepsy symptoms measured by the PGI-C | narcolepsy symptoms as measured by the PGI-C |
| scale. | at Week 12 in the targeted patient |
| | population. |
| | Endpoint: PGI-C score at Week 12 |
| | Population level summary: The proportion of |
| | participants reporting much or very much |
| | improved overall narcolepsy symptoms will be |
| | estimated for each treatment group and |
| | comparison to placebo will be based on the odds |
| | ratio of reporting much or very much improved |
| | overall narcolepsy symptoms between treatment |
| | groups. |
| Secondary objective: | Secondary Estimand: The treatment effect of |
| To assess the effect of Compound A on overall | Compound A compared to placebo on overall |
| severity of narcolepsy symptoms measured by the | severity of narcolepsy symptoms as measured by |
| NSS-CT. | the NSS-CT at Week 12 in the targeted patient |
| | population. |
| | Endpoint: NSS-CT total score at Week 12 |
| | Population level summary: The mean of the |
| | variable will be estimated for each treatment |
| | group and comparison to placebo will be based on |
| | the difference in means between treatment groups. |
| Secondary objective: | Secondary Estimand: The treatment effect of |
| To assess the efficacy of Compound A on | Compound A compared to placebo on functional |
| functional impacts of narcolepsy as assessed by | impacts of narcolepsy as assessed by the FINI at |
| the FINI. | Week 12 in the targeted patient population. |
| | Endpoints: FINI domain scores at Week 12 for: |
| | Tiredness, Cognitive Functioning, Cataplexy, |
| | Social Activities, Everyday Activities, and |
| | Everyday Responsibilities |
| | Population level summary: The mean of the |
| | score for each domain will be estimated for each |
| | treatment group and comparison to placebo for |
| | each domain will be based on the difference in |
| | means between treatment groups. |
| Secondary objective: | Secondary Estimand: The treatment effect of |
| To assess the effect of Compound A on quality of | Compound A compared to placebo on quality of |
| life of participants, as assessed by the SF-36. | life of participants as assessed by the SF-36 at |
| | Week 12 in the targeted patient population. |
| | Endpoints: SF-36 component summary scores at |
| | Week 12 for physical and mental components |
| | Population level summary: The mean of the |
| | score for each component will be estimated for |
| | each treatment group and comparison to placebo |
| | for each component will be based on the |
| | difference in means between treatment groups. |

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A method of treating narcolepsy type 1 (NT1), the method comprising administering to a subject in need thereof about 1 mg to about 7 mg of Compound A: 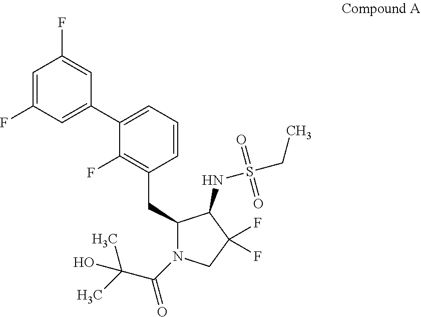 [SMILES: CCS(=O)(=O)N[C@@H]1[C@H](Cc2cccc(-c3cc(F)cc(F)c3)c2F)N(C(=O)C(C)(C)O)CC1(F)F]
or an equivalent amount of a pharmaceutically acceptable salt thereof.

2. The method of claim 1, wherein the subject in need thereof is administered two daily doses of Compound A, wherein each dose is from about 0.5 mg to about 5 mg, or an equivalent amount of a pharmaceutically acceptable salt thereof, and wherein the doses are administered about 2.5 hours to about 5.5 hours apart.

3. A method of treating blunted nocturnal blood pressure (BP) dip in a subject having narcolepsy type 1 (NT1), the method comprising administering to the subject about 1 mg to about 7 mg of Compound A: 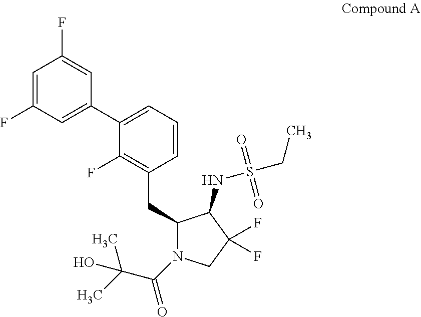 [SMILES: CCS(=O)(=O)N[C@@H]1[C@H](Cc2cccc(-c3cc(F)cc(F)c3)c2F)N(C(=O)C(C)(C)O)CC1(F)F]
or an equivalent amount of a pharmaceutically acceptable salt thereof.

4. The method of claim 3, wherein the subject in need thereof is administered two daily doses of Compound A, wherein each dose is from about 0.5 mg to about 5 mg, or an equivalent amount of a pharmaceutically acceptable salt thereof, and wherein the doses are administered about 2.5 hours to about 5.5 hours apart.

5. A method of treating cognitive impairment in a subject having narcolepsy type 1 (NT1), the method comprising administering to the subject about 1 mg to about 7 mg of Compound A: 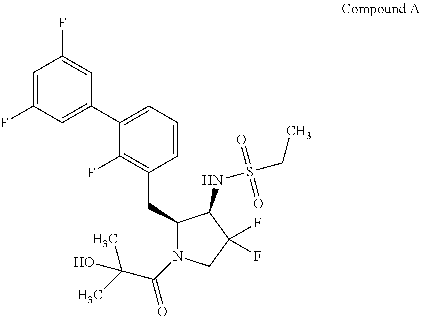 [SMILES: CCS(=O)(=O)N[C@@H]1[C@H](Cc2cccc(-c3cc(F)cc(F)c3)c2F)N(C(=O)C(C)(C)O)CC1(F)F]
or an equivalent amount of a pharmaceutically acceptable salt thereof.

6. The method of claim 5, wherein the subject in need thereof is administered two daily doses of Compound A, wherein each dose is from about 0.5 mg to about 5 mg, or an equivalent amount of a pharmaceutically acceptable salt thereof, and wherein the doses are administered about 2.5 hours to about 5.5 hours apart.

7. The method of claim 2, wherein the two daily doses of Compound A, or the equivalent amount of a pharmaceutically acceptable salt thereof, are administered about 3 hours apart, about 3.5 hours apart, about 4 hours apart, about 4.5 hours apart, or about 5 hours apart.

8.-11. (canceled)

12. method of claim 2, wherein each of the two daily doses is about 1 mg to about 2 mg.

13. The method of claim 2, wherein each of the two daily doses is about 1 mg.

14. The method claim 2, wherein each of the two daily doses is about 2 mg.

15. The method of claim 1, wherein Compound A, or the pharmaceutically acceptable salt thereof, is administered orally.

16. The method of claim 15, wherein Compound A, or the pharmaceutically acceptable salt thereof, is administered as a tablet.

17. The method of claim 1, wherein Compound A, or the pharmaceutically salt thereof, is administered daily over a period of about 4 weeks to about 24 weeks.

18. The method of claim 17, wherein Compound A, or the pharmaceutically salt thereof, is administered daily over a period of about 12 weeks.

19. The method of claim 1, wherein Compound A, and not a pharmaceutically acceptable salt thereof, is administered to the subject.

20. The method of claim 1, wherein the subject experiences a higher mean sleep latency from the Maintenance of Wakefulness Test (MWT) than baseline after a treatment period.

21.-23. (canceled)

24. The method of claim 1, wherein the subject experiences a lower Epworth Sleepiness Scale (ESS) total score after a treatment period compared to baseline.

25.-27. (canceled)

28. The method of claim 1, wherein the subject experiences a lower weekly cataplexy rate (WCR) after a treatment period compared to baseline.

29.-41. (canceled)

42. The method of claim 1, wherein the subject experiences an increase after a treatment period in one or more of:
(a) systolic dip amplitude; and
(b) diastolic dip amplitude;
compared to an untreated subject.

43. (canceled)

44. The method of claim 1, wherein the subject experiences a reduction in attentional impairment compared to baseline.

45.-47. (canceled)
